(19) United States
(12) Patent Application Publication
(10) Pub. No.: US 2016/0208016 A1
(43) Pub. Date: Jul. 21, 2016

(54) Anti-ADAM12 antibodies for the treatment of cancer

(71) Applicant: UNIVERSITY OF COPENHAGEN, Copehagen K (DK)

(72) Inventors: Reidar Albrechtsen, Klampenborg (DK); Camilla Fröhlich, Copenhagen Ø (DK); Ulla Wewer, Klampenborg (DK)

(21) Appl. No.: 14/915,182

(22) PCT Filed: Aug. 29, 2014
(86) PCT No.: PCT/DK2014/050257

§ 371 Date: Feb. 26, 2016

(30) Foreign Application Priority Data
Aug. 29, 2013 (DK) PA 2013 00488

(51) Int. Cl. C07K 16/40; C12Q 1/68; G01N 33/577

(52) U.S. Cl. CPC C07K 16/40; G01N 33/577; C12Q 1/6886; C07K 2317/54; C07K 2317/55; C07K 2317/56; A61K 2039/505

(57) ABSTRACT

The present disclosure concerns monoclonal antibodies directed to the pro-domain of ADAM 12 and their use in the treatment of cancer.

### FIELD OF INVENTION

The present invention relates to domain specific anti-ADAM12 antibodies and their therapeutic use in the treatment of cancer.

### BACKGROUND OF INVENTION

Cancer is the second most common cause of disease-related death in Western countries. Despite improved screening for early detection as well as improved treatment modalities, there is still an urgent need for development of new treatments. Personalized treatment is expected to be the future cancer treatment, and one part of that is the development of targeted therapy drugs. Enzymes are key molecules regulating cancer cell behaviour and several enzymes are currently being targeted as intervention points in the quest for finding new cancer treatments.

MMPs (matrix metalloproteinases), in particular MMP-14, play a key role in various aspects of cancer pathology, including tumour growth, dissemination, and angiogenesis. MMP-14 is a classical transmembrane metalloprotease and it is upregulated in human cancer and accelerates tumour progression in mouse models of cancer. MMP-14 degrades extracellular-matrix components (i.e. fibrillar collagen and gelatin), activates enzymes such as MMP-2 and MMP-13, sheds cell-surface proteins, and was recently shown to prevent collagen-induced apoptosis. Due to the high impact of metalloproteases in several pathological processes a variety of small-molecule inhibitors targeting proteolytic activities have been developed (Fingleton, The Cancer Degradome, 2008). However, these inhibitors failed in clinical trials because of lack of efficacy and significant toxicity, possibly due to a high degree of structural similarity in the catalytic site of the metalloproteases (Coussens, 2002).

Other strategies have emerged to reduce toxicity, such as the use of monoclonal antibodies, which can target the protein of interest with higher specificity, as potential treatments for several diseases, including cancer (Fang, 2011). Indeed, companies and research laboratories have developed antibodies against MMP-14 and these were found to block tumour growth, invasion and angiogenesis in xenograft models of cancer (Devy, 2009). However, one problem that may occur is that MMP-14 is widely expressed and is involved in cleavage and shedding of a wide range of physiologically important molecules of the extracellular matrix and membrane-anchored proteins, thus there is a risk that “pleiotropic-like”, unwanted side effects may occur. Thus there is a need for new methods for treating cancer, with reduced toxicity and less unwanted side effects.

### SUMMARY OF INVENTION

ADAMs (A Disintegrin And Metalloproteases) belong together with the MMPs to the metzincin subclan of zink-dependent metalloproteases (Stocker and Bode, 1995). The ADAM12 gene was cloned by the inventors (Gilpin et al., 1998). ADAM12 exhibits a limited tissue distribution in normal tissue, but is expressed during excessive growth, such as in the placenta, during muscle regeneration, and in particular in cancerous tissue. In fact, ADAM12 is highly upregulated in a variety of human cancers including breast, bladder, laryngeal, and lung carcinomas, as well as in glioblastomas (Kveiborg et al., 2008). Overexpression of ADAM12 accelerates tumour cell proliferation, inhibits tumour cell apoptosis, and increases tumour cell migration and invasion.

Two naturally occurring human ADAM12 splice variants exist, which were named ADAM12-L and ADAM12-S. The ADAM12-L domain compositions resemble the prototypical transmembrane ADAM protein, containing a prodomain, metalloprotease, disintegrin and cysteine-rich domains, followed by a transmembrane domain and a cytoplasmic tail domain. ADAM12-S, the soluble splice variant, contains the same domains as ADAM12-L, but lacks the transmembrane domain and the cytoplasmic tail is replaced by a stretch of 33 amino acids in the C-terminus. ADAM12-L stimulates tumour growth independent of its catalytic activity in the PyMT mouse model of breast cancer (Frohlich et al., 2011).

Through its disintegrin and cysteine-rich domains, the ADAM12 molecule is involved in cell adhesion via binding to integrins and syndecan. In particular, the inventors and others have shown that the disintegrin domain of ADAM12-S interacts with integrins. The prodomain, approximately 26 kDa, is thought to primarily keep the ADAM12 molecule in an inactive form until cleavage at the furin-site between the prodomain and the catalytic domain. Interestingly, after furin cleavage the prodomain remains associated with the mature molecule through non-covalent bonds (Wewer et al., 2006).

The present inventors have found that the MMP-14 proteolytic activity enhanced via ADAM12 could be significantly reduced by monoclonal antibodies directed against ADAM12. Indeed, the antibodies provided herein are capable of relieving the inhibition of apoptosis of tumour cells. It is thus an object of the present invention to provide antibodies which are directed against ADAM12 and which can be used for treating cancer. Also provided is a method of treating cancer by administration of such antibodies.

In one aspect the invention relates to an antibody capable of specifically binding an epitope within the prodomain of ADAM12 (SEQ ID NO: 2), said antibody selected from the group consisting of:
i the monoclonal antibody produced by the hybridoma cell line 7B8 deposited under the Budapest Treaty with HPA Cultures collection (ECACC) under accession number 13082101;
ii the monoclonal antibody produced by the hybridoma cell line 8F8 deposited under the Budapest Treaty with HPA Cultures collection (ECACC) under accession number 13082102;
iii an antibody capable of binding the same epitope as 7B8 or 8F8;
iv an antibody capable of inhibiting binding of 7B8 or 8F8 to ADAM12;
v an antibody having the V_{H} and V_{L} of 7B8 or 8F8;
vi an antibody having the CDRs of the V_{H} and V_{L} of 7B8 or 8F8.

In another aspect the invention relates to an antibody for use as a medicament.

In another aspect the invention relates to an antibody or a functional equivalent thereof, capable of specifically recognising and binding an epitope within the prodomain of ADAM12 (SEQ ID NO: 2), wherein said antibody or functional equivalent thereof specifically recognises at least part of an epitope recognised by one or more reference antibodies selected from the group consisting of:
i the monoclonal antibody produced by the hybridoma cell line 7B8 deposited under the Budapest Treaty with HPA Cultures collection (ECACC) under accession number 13082101;
ii the monoclonal antibody produced by the hybridoma cell line 8F8 deposited under the Budapest Treaty with HPA Cultures collection (ECACC) under accession number 13082102;
for use in a method of treatment of cancer in a subject.

In another aspect the invention relates to a method of treatment of cancer in an individual in need thereof, the method comprising the steps of:
a) providing a sample from tumour tissue of an individual,
b) determining the expression level of ADAM12 in the sample of step a),
c) correlating the expression level of step b) with the expression level of a control tissue,
d) assessing a treatment regime,
e) administering to the individual a therapeutically effective amount of an antibody as disclosed herein.

In yet another aspect the invention relates to a method of treatment of cancer in an individual in need thereof, the method comprising the steps of:
a) providing a sample from tumour tissue of an individual,
b) determining the degradation level of gelatin in the sample of step a,
c) correlating the expression level of step b with the expression level of a control tissue,
d) assessing a treatment regime,
e) administering to the individual a therapeutically effective amount of an antibody as disclosed herein.

In yet another aspect the invention relates to a method of treatment of cancer in an individual in need thereof, said method comprising administering an antibody which inhibits gelatin degradation.

In yet another aspect the invention relates to a method of treatment of cancer in an individual in need thereof, said method comprising administering an antibody directed against the prodomain of ADAM12.

In yet another aspect the invention relates to the use of the antibody as disclosed herein for the preparation of a medicament for the treatment of cancer.

In yet another aspect the invention relates to an antibody capable of selectively recognising and binding the antibody as disclosed herein.

In yet another aspect the invention relates to a method of inhibiting formation of a complex between ADAM12, MMP-14 and/or αVβ3, said method comprising administering the antibody as disclosed herein.

In yet another aspect the invention relates to a method for producing the antibody as disclosed herein, comprising the steps of: administering to a mammal a protein comprising the prodomain of ADAM12 or a fragment thereof or a functional equivalent thereof, screening for the ability of said antibody to bind to the prodomain of ADAM12; screening for the ability of said antibody to inhibit the formation of a complex between MMP-14 and/or αVβ3.

In yet another aspect the invention relates to a method for producing the antibody of the invention, comprising the steps of transfecting a host cell with a nucleic acid construct encoding said antibody.

In yet another aspect the invention relates to a pharmaceutical composition comprising the antibody as disclosed herein.

In yet another aspect the invention relates to a kit comprising the pharmaceutical composition as disclosed herein, and instructions for use.

### OVERVIEW OF DRAWINGS

FIG. 1. Recruitment of MMP-14 to the cell surface is stimulated by ADAM12.

FIG. 2: ADAM12 and MMP-14 stainings.

FIG. 3: ADAM12 and MMP-14 colocalization at the cell surface induces gelatin degradation.

FIG. 4. Gelatin degradation is increased in cells expressing ADAM12 at the cell surface.

FIG. 5. ADAM12-induced gelatin degradation is mediated by MMP-14.

FIG. 6. ADAM12-induced gelatin degradation is dependent on αVβ3 integrin.

FIG. 7. Expression of β3 integrin in 293-Vnr direct endogenous MMP-14 to the cell surface.

FIG. 8. Monoclonal antibodies (mAbs) against ADAM12 inhibit ADAM12-induced gelatin degradation.

FIG. 9. Monoclonal antibodies (mAbs) against ADAM12 inhibit ADAM12-induced gelatin degradation.

FIG. 10. 7B8 and 8F8 recognise the prodomain of ADAM12.

FIG. 11. ADAM12 protects tumour cells against apoptosis in vitro.

FIG. 12. ADAM12 protects tumour cells against apoptosis in vitro

FIG. 13. ADAM12 accelerates tumour growth and inhibits tumour apoptosis in vivo.

FIG. 14. Positive correlation between expression of ADAM12 and expression of MMP-14 and MMP-2.

FIG. 15. 8F8 has no effect on tumour cell growth in vitro.

### DETAILED DESCRIPTION OF DRAWINGS

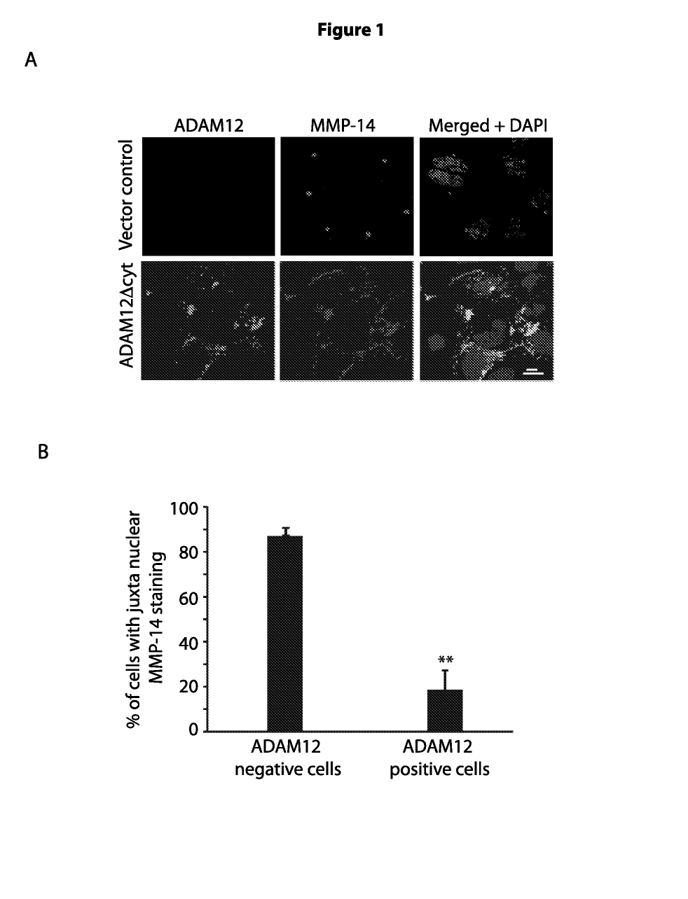

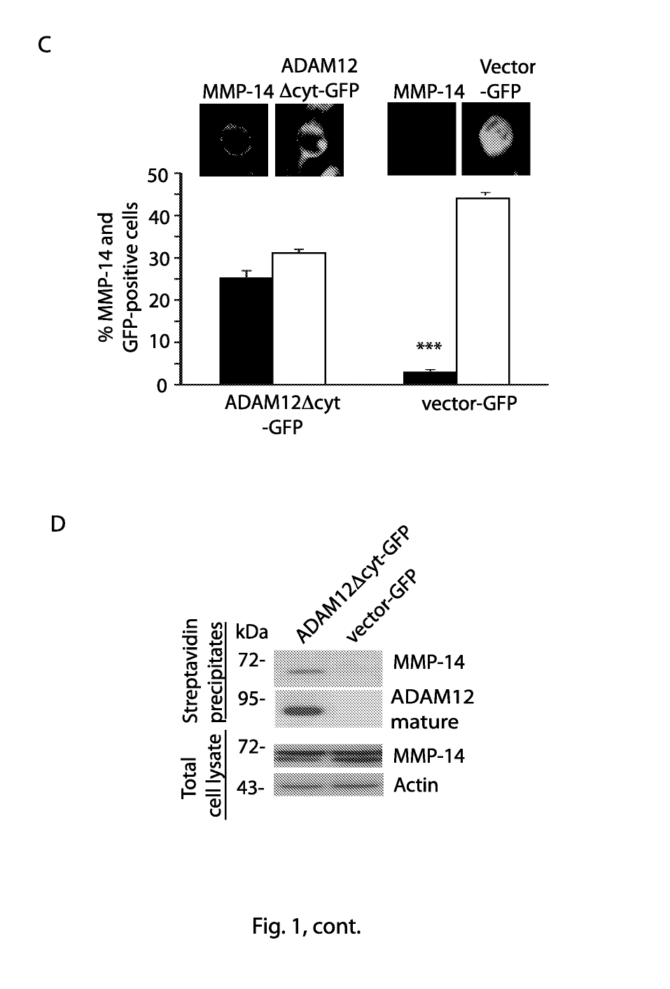

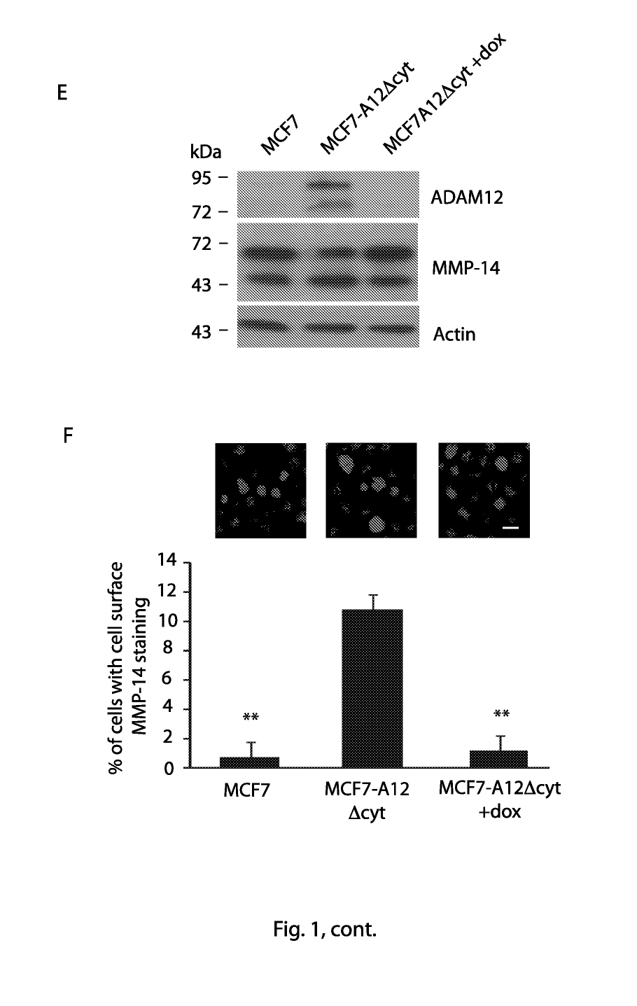

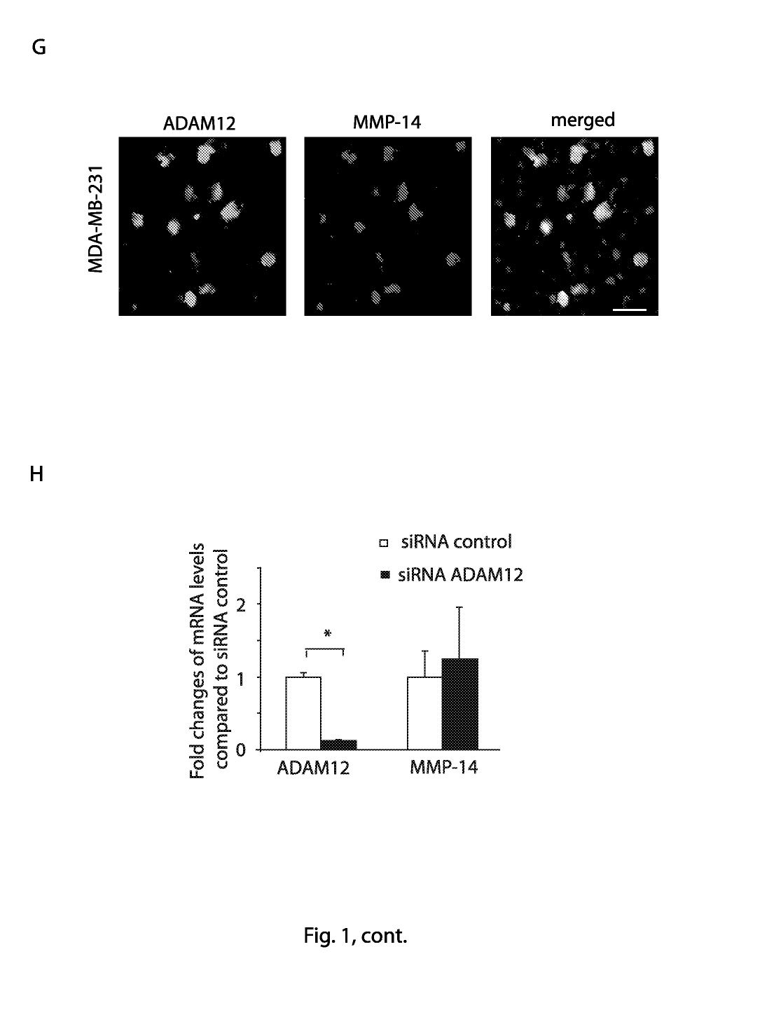

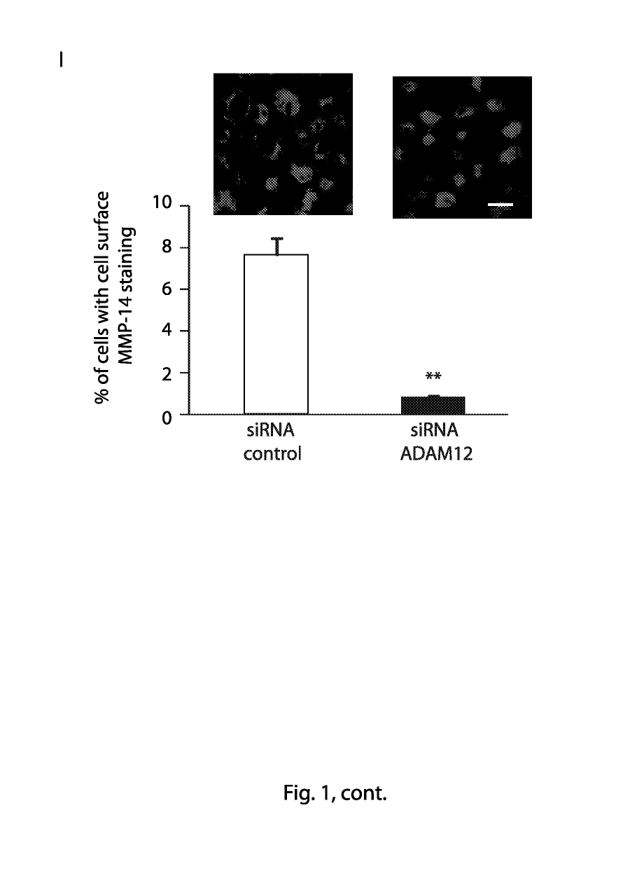

FIG. 1. Recruitment of MMP-14 to the Cell Surface is Stimulated by ADAM12.

(A-D) 293-VnR cells were transfected with either vector control or ADAM12Δcyt-GFP. (A) Cells were stained for MMP-14 (red), ADAM12 (green) and the nucleus (blue). Scale bar=6 μm. (B) Cells were stained as described in (A) and >500 cells per experiment were counted for localization of MMP-14 to the juxta nuclear region. Mean data (±standard deviation; s.d.) are expressed in percentage of total cells. (C) Cytospin experiments detected surface MMP-14 in 293-VnR cells. The graph shows the distribution of MMP-14 and GFP cells in mean percentage (±s.d.) of total cells. For each experiment, more than 1000 cells were counted. (D) Cell surface biotinylation assay. Streptavidin precipitates were analyzed for MMP-14 and ADAM12Δcyt-GFP by Western blot. MMP-14 protein is also shown in total cell lysates. (E) Total cell lysates from wild-type MCF7 cells, MCF7-A12Δcyt, MCF7-A12Δcyt+dox were analyzed for MMP-14 and ADAM12 protein levels by Western blot. (F) Cytospin of MCF7 cells were immunostained for cell-surface MMP-14 and counted as described in (C). Mean data (±s.d.) are expressed in percentage of total cells. (G) Cytospin of MDA-MB-231 cells immunostained for cell-surface ADAM12 (green), MMP-14 (red), and DAPI (blue). Scale bar=12 μm. (H) MDA-MD-231 cells were treated with ADAM12 siRNA or control siRNA for 48 hours, mRNA was extracted, and then subjected to qPCR to detect ADAM12 and MMP-14. (I) Cytospin of siRNA-treated MDA-MB-231 cells immunostained and counted as described in (C). Mean data (±s.d.) are expressed in percentage of total cells. *P<0.05, **P<0.01, ***P<0.001, Student's t-test.

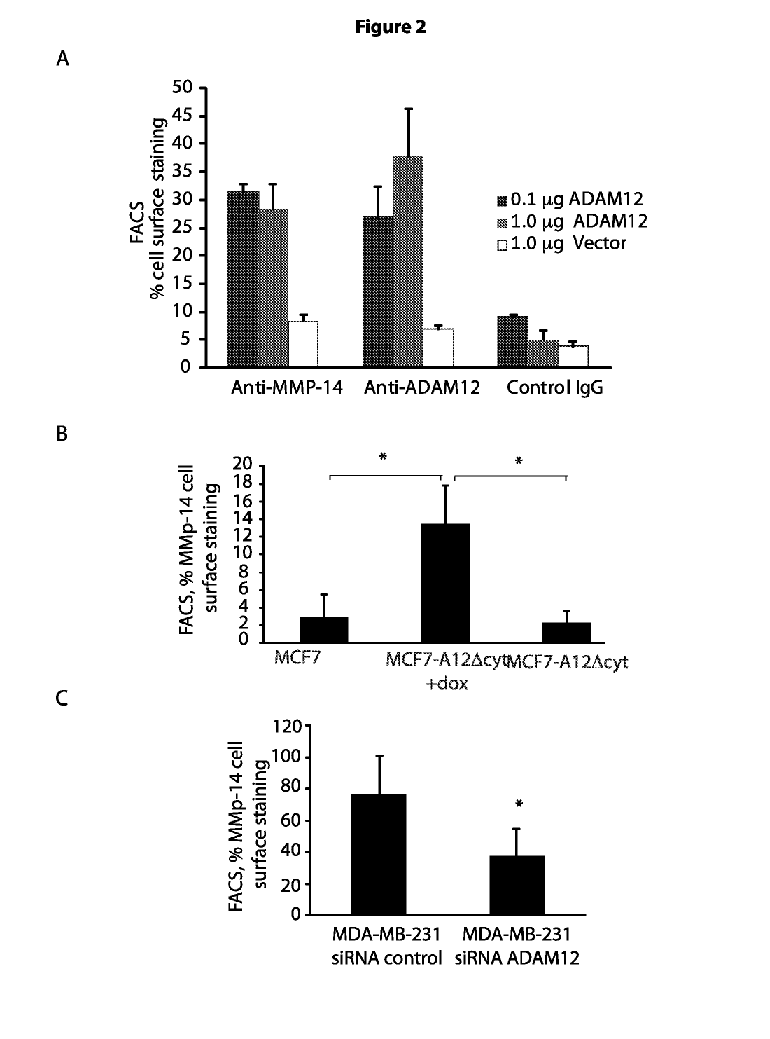

FIG. 2. ADAM12 and MMP-14 Stainings.

(A) ADAM12 and MMP-14 stainings on the cell surface of 293-VnR cells transfected with 0.1 or 1 μg ADAM12 or 1 μg control plasmid were analyzed by FACS. Data are expressed as the mean percentage of cells with cell surface staining. (B,C) MMP-14 staining on the cell surface of non-permeabilized MCF7 cells and MDA-MB-231 were analyzed by FACS. Data are expressed as the mean percentage of cells with cell surface staining.

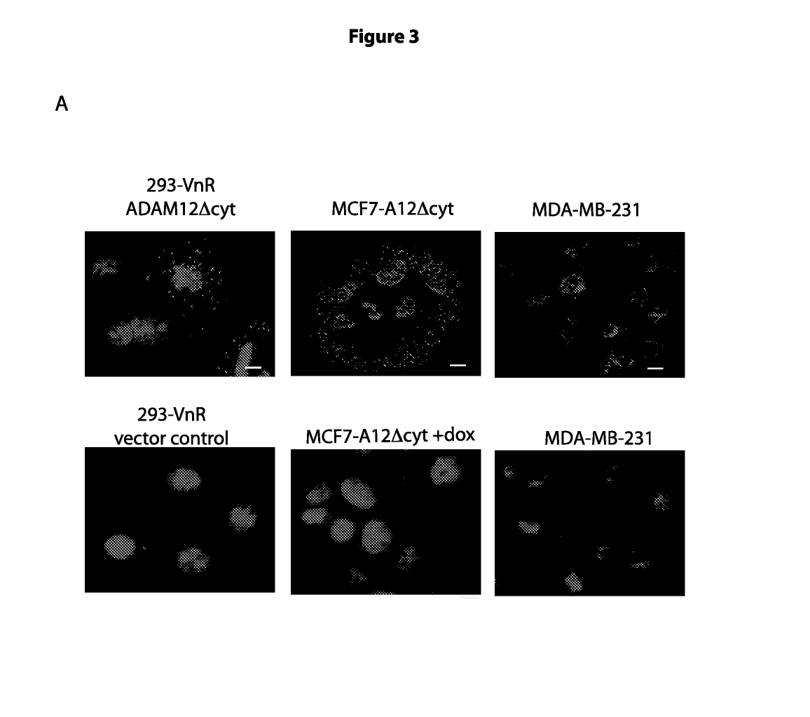

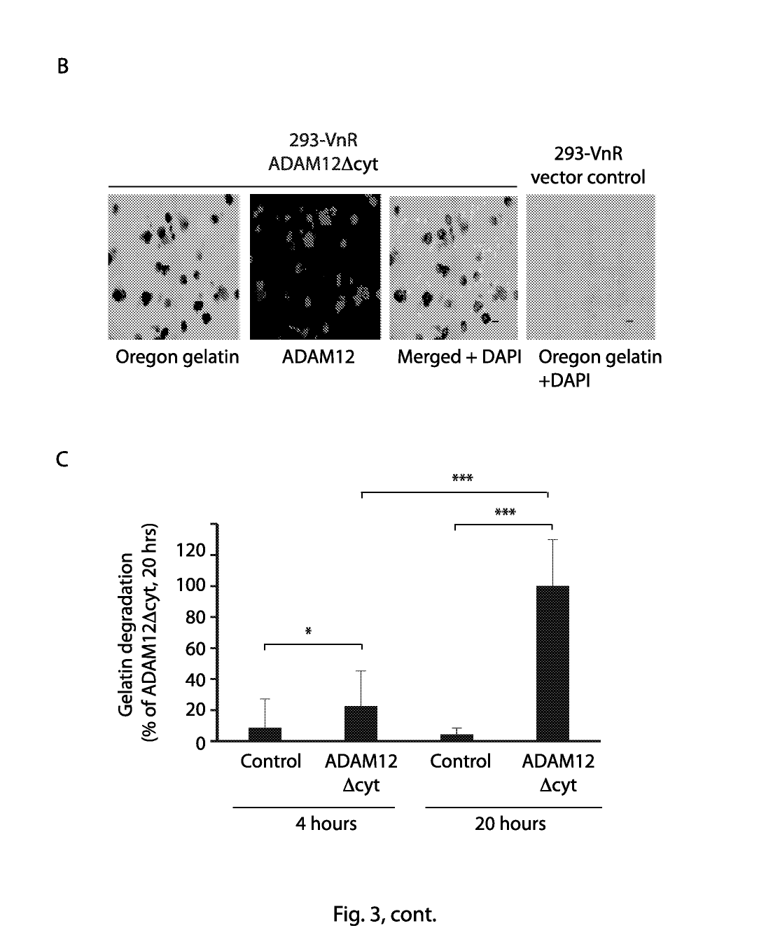

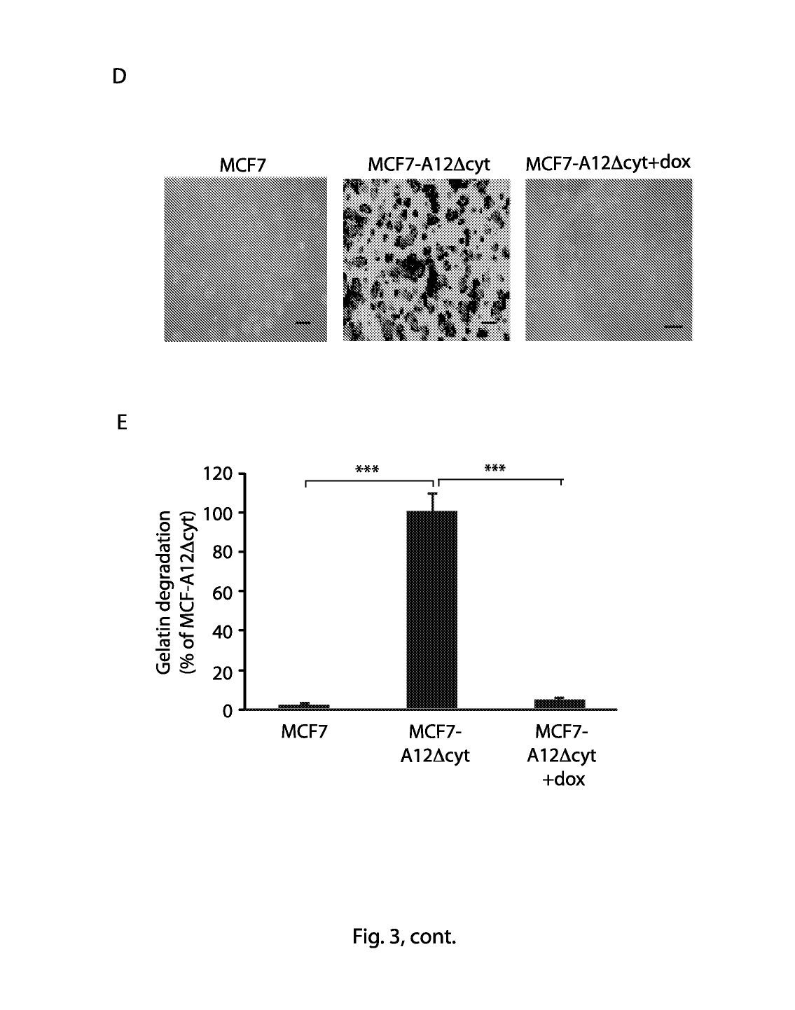

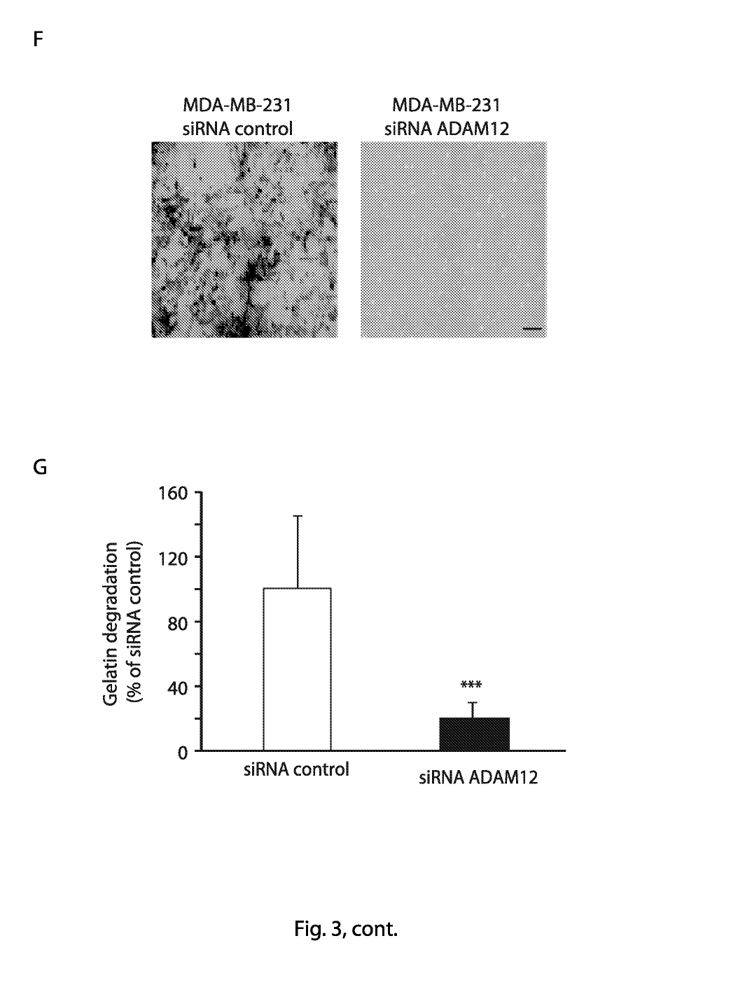

FIG. 3. ADAM12 and MMP-14 Colocalization at the Cell Surface Induces Gelatin Degradation.

(A) Non-permeabilized 293-VnR cells transfected with ADAM12Δcyt or control vector (left panels), MCF7-A12Δcyt and MCF7-A12Δcyt+dox cells (middle panels), and cytospin of MDA-MB-231 cells (right panels) were subjected to Duolink® reagents with antibodies to ADAM12 (6E6) and MMP-14. The brighter spots indicate colocalization. Left and middle panels, scale bar=8 μm; right panels, scale bar=12 μm. (B) In situ solid-phase gelatinase assay in 293-VnR cells transfected with vector control or ADAM12Δcyt. Cell cultures were stained for ADAM12 and the nucleus and tested for gelatin degradation. Scale bar: Vector control=100 μm, and ADAM12Δcyt=40 μm. (C) Percentage of gelatin degradation after 4 and 20 hours. Areas without green fluorescence were measured (μm²) from experiments in (B) and the gelatin degradation per cell was calculated. Mean percentage data (±s.d.) are expressed relative to the mean ADAM12Δcyt degradation percentage value at 20 hour (set at 100%). (D) In situ solid-phase gelatinase assay of MCF7, MCF7-A12Δcyt, and MCF7-A12Δcyt+dox cells. Scale bar; middle image=100 μm; otherwise=20 μm. (E) Mean percentage of gelatin-degradation (±s.d.) data for the MCF7 cells shown in (D) are expressed relative to the mean MCF7-A12Δcyt degradation value (set a 100%). (F) In situ solid phase gelatinase assay (20 hours) of MDA-MB-231 cells treated with control siRNA or ADAM12 siRNA. Scale bar=10 μm. (G) Mean percentage of gelatin-degradation (±s.d.) data for the MDA-MB-231 cells shown in (F) are expressed relative to the mean siRNA control degradation value (set at 100%). *P<0.05, **P<0.01, ***P<0.001, Student's t-test.

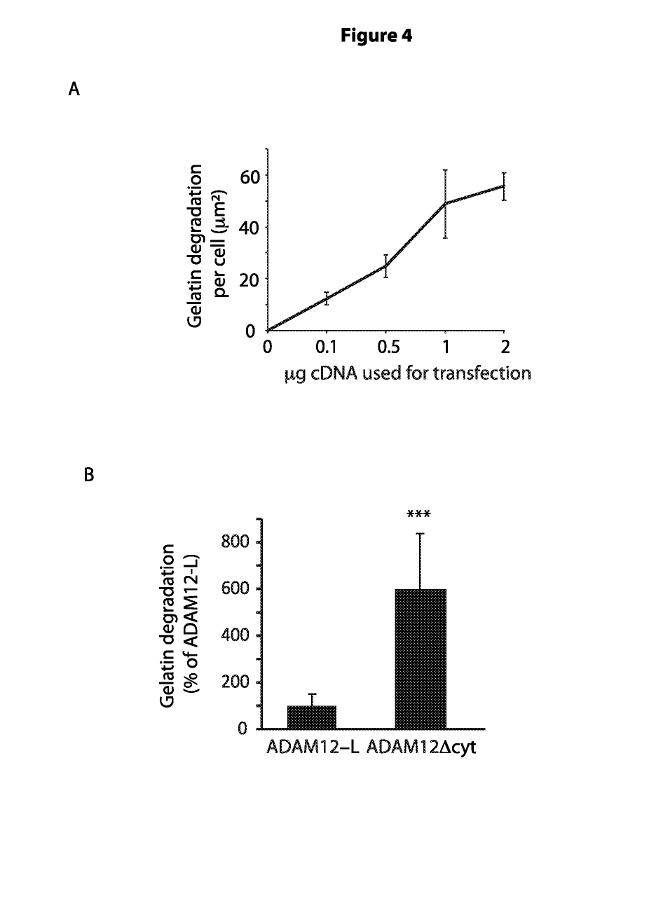

FIG. 4. Gelatin Degradation is Increased in Cells Expressing ADAM12 at the Cell Surface.

(A) 293-VnR cells were transfected with ADAM12Δcyt and coated on gelatin coupled to Oregon Greene 488 dye (10 μg/ml). Mean gelatin degradation per cell (±s.d.) in 293-VnR cells transfected with 0.1, 0.5, 1, or 2 μg ADAM12Δcyt cDNA plasmid. (B) In situ solid-phase gelatinase assay of 293-VnR cells transfected with ADAM12-L or ADAM12Δcyt. Mean data (±s.d.) are expressed relative to the mean ADAM12-L degradation value (set at 100%). ***P<0.001, Student's t-test.

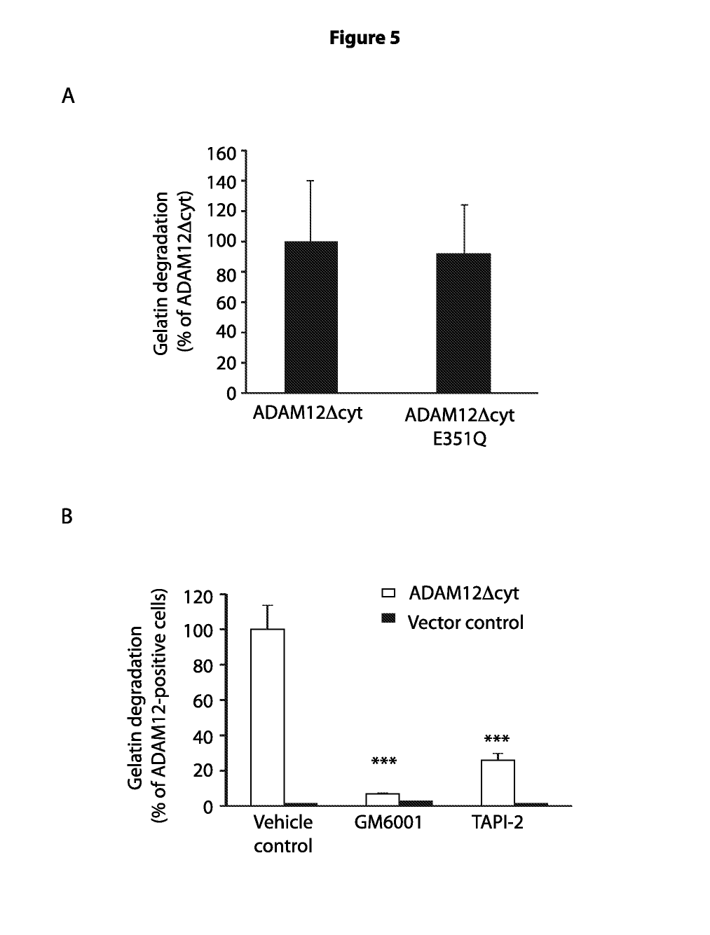

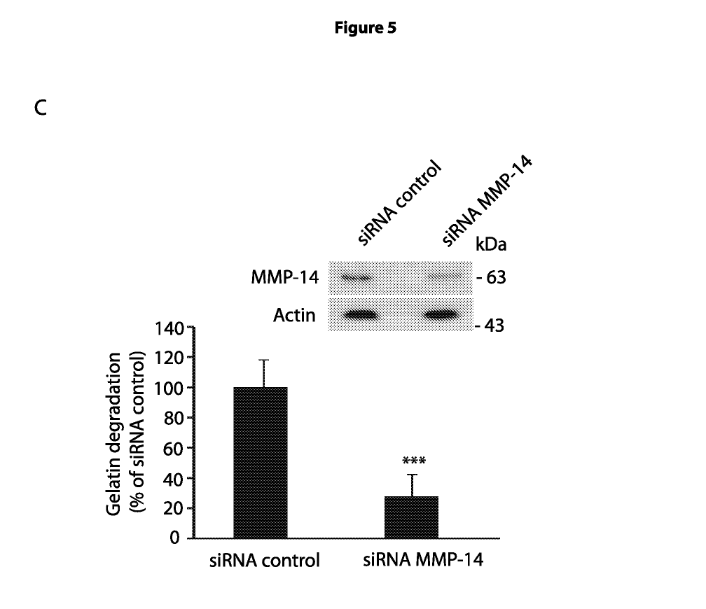

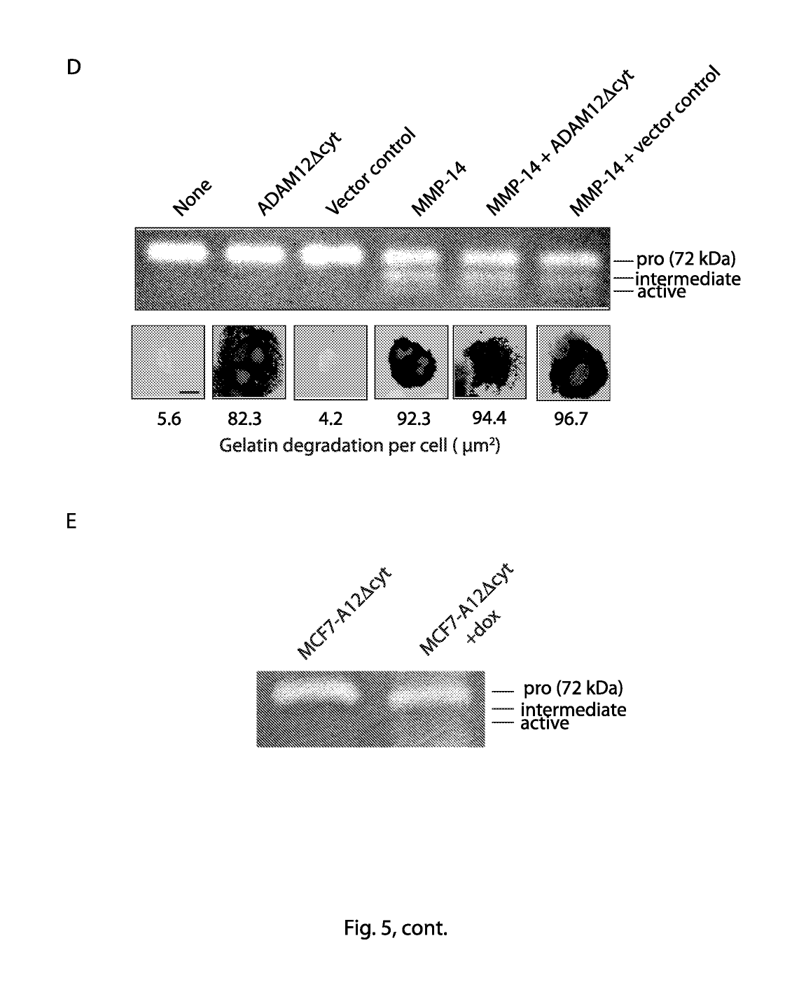

FIG. 5. ADAM12-Induced Gelatin Degradation is Mediated by MMP-14.

(A) In situ solid phase gelatinase assay using 293-VnR transfected with ADAM12Δcyt or its catalytic inactive form ADAM12Δcyt-E351Q. Data are expressed as the mean gelatin degradation (±s.d.) relative to the mean ADAM12Δcyt degradation value (set at 100%). (B) In situ solid-phase gelatinase assay of 293-VnR cells transfected with ADAM12Δcyt or control vector and treated with GM6001 (10 μM), TAPI-2 (10 μM), or vehicle only (control) overnight. Cells were stained for ADAM12 and for the nucleus (DAPI). The mean number of gelatin-degrading ADAM12-positive cells (±s.d.) is presented as a percentage of the total number of ADAM12-positive cells. (C) In situ solid-phase gelatinase assay of 293-VnR cells cotransfected with ADAM12Δcyt and control or MMP-14 siRNA. Mean data (±s.d.) are expressed relative to the mean siRNA control degradation value (set at 100%). Inset, Western blots of total cell lysates analyzed for MMP-14. (D) MMP-2 zymography for untransfected 293-VnR cells or 293-VnR cells transfected with ADAM12Δcyt, vector control, or MMP-14, or cotransfected with MMP-14 and ADAM12Δcyt or MMP-14 and vector control. The images in the lower panel illustrate gelatin degradation and nuclear staining (DAPI) in the respective cultures. Gelatin degradation was quantified by measuring the degraded area in μm² and correlated to the number of cells. Scale bar=10 μm. ***P<0.001, Student's t-test.

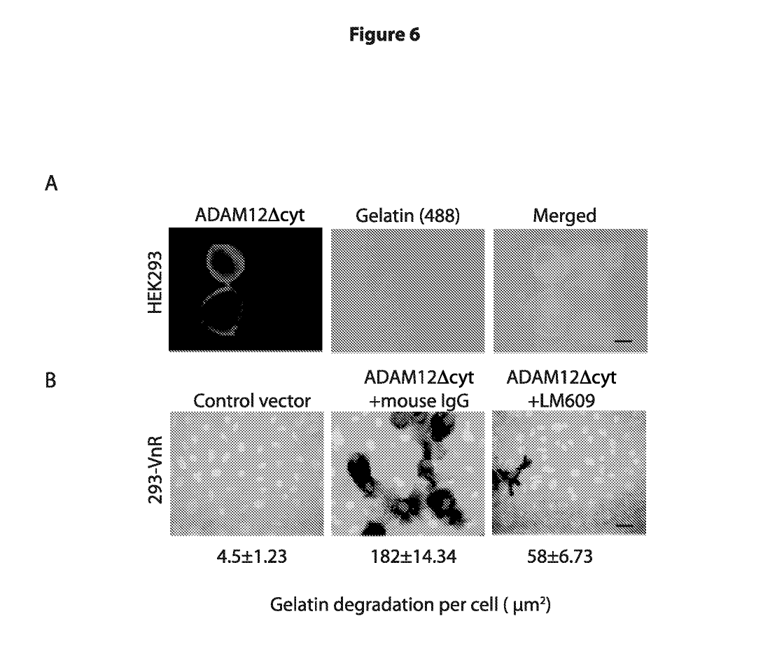

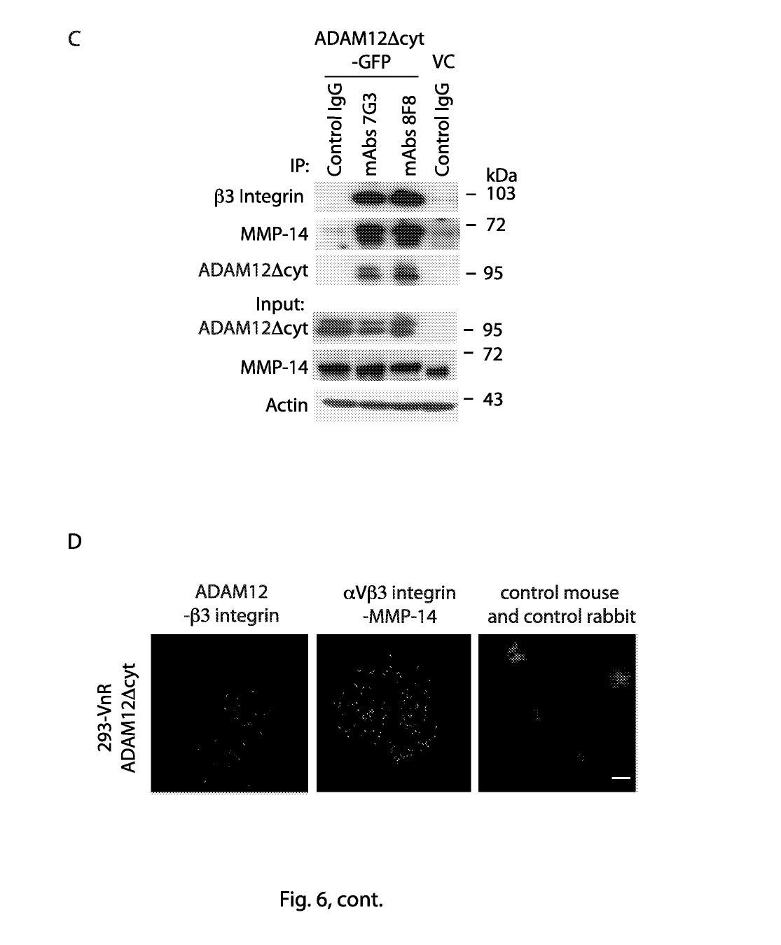

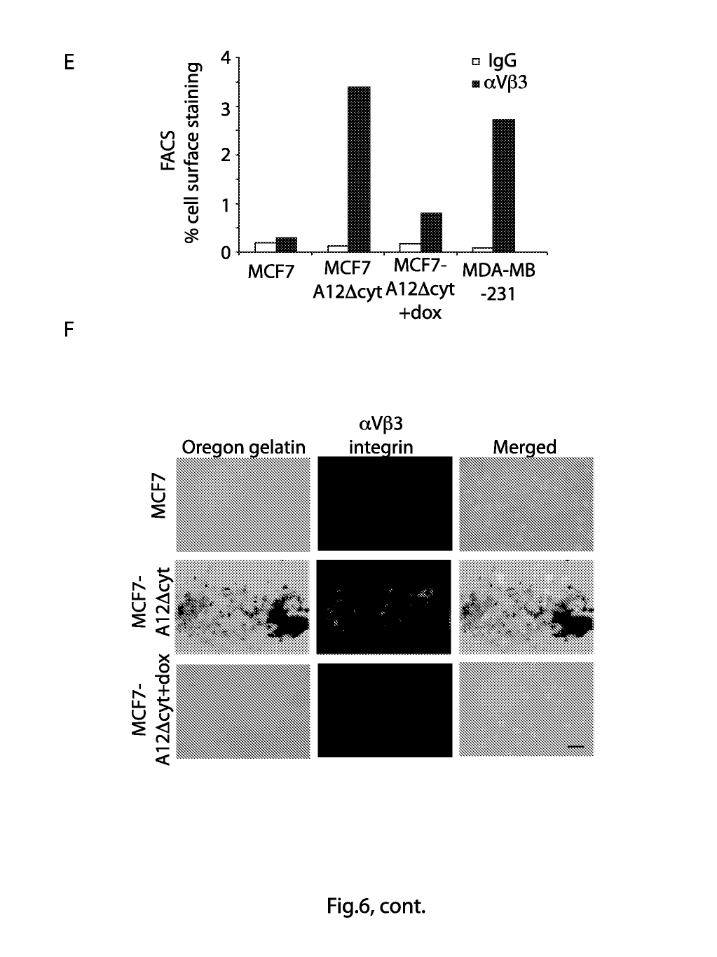

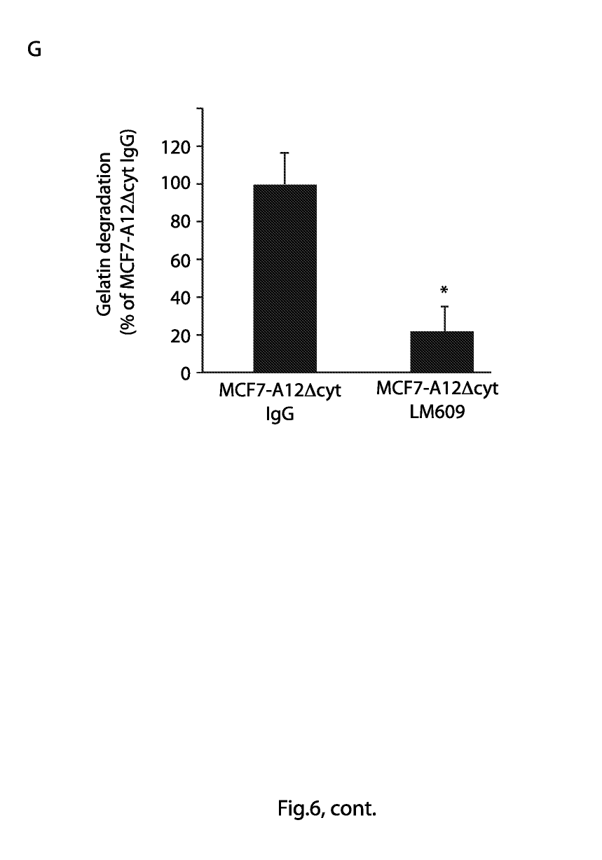

FIG. 6. ADAM12-Induced Gelatin Degradation is Dependent on αVβ3 Integrin.

(A) In situ solid-phase gelatinase assay of HEK293 cells transfected with ADAM12Δcyt and immunostained for ADAM12, and DAPI staining. Scale bar=8 μm. (B) In situ solid-phase gelatinase assay of 293-VnR cells transfected with ADAM12Δcyt or control vector and treated overnight with normal mouse IgG or an inhibitory antibody against αVβ3 integrin (10 μg/ml LM609). The mean gelatin degradation per cell (μm²) (±s.d.) is presented below the images. Scale 801 bar=20 μm. (C) 293-VnR cells were transfected with ADAM12Δcyt-GFP or vector control, immunoprecipitation mouse IgG or mAbs against ADAM12 (7G3 or 8F8). Precipitates and input samples were analysed by Western blotting with indicated antibodies. (D) 293-VnR cells transfected with ADAM12Δcyt were subjected to Duolink® reagents with antibodies to ADAM12 (6E6)/133 integrin, αVβ3 integrin/MMP-14, and negative control mouse and rabbit IgG. Scale bar=8 μm. (E) Mean percentage cell-surface staining of αVβ3 integrin (LM609 antibody staining) for MCF7, MCF7-A12Δcyt, MCF7-A12Δcyt+dox, and MDA-MB-231 cell lines analyzed by FACS. Normal mouse IgG was used as a staining control. (F) In situ solid-phase gelatinase assay of MCF7, MCF7-A12Δcyt, and MCF7-A12Δcyt+dox. Cells were immunostained for αVβ3 integrin (LM609 antibody) and the nuclei visualized by DAPI staining. Scale bar=15 μm. (G) Mean percentage of gelatin degradation (±s.d.) for MCF7-A12Δcyt cells were treated overnight with normal mouse IgG or inhibitory αVβ3 integrin antibody (LM609), expressed relative to the mean MCF7-A12Δcyt cells+IgG degradation value (set at 100%). *P<0.001, Student's t-test.

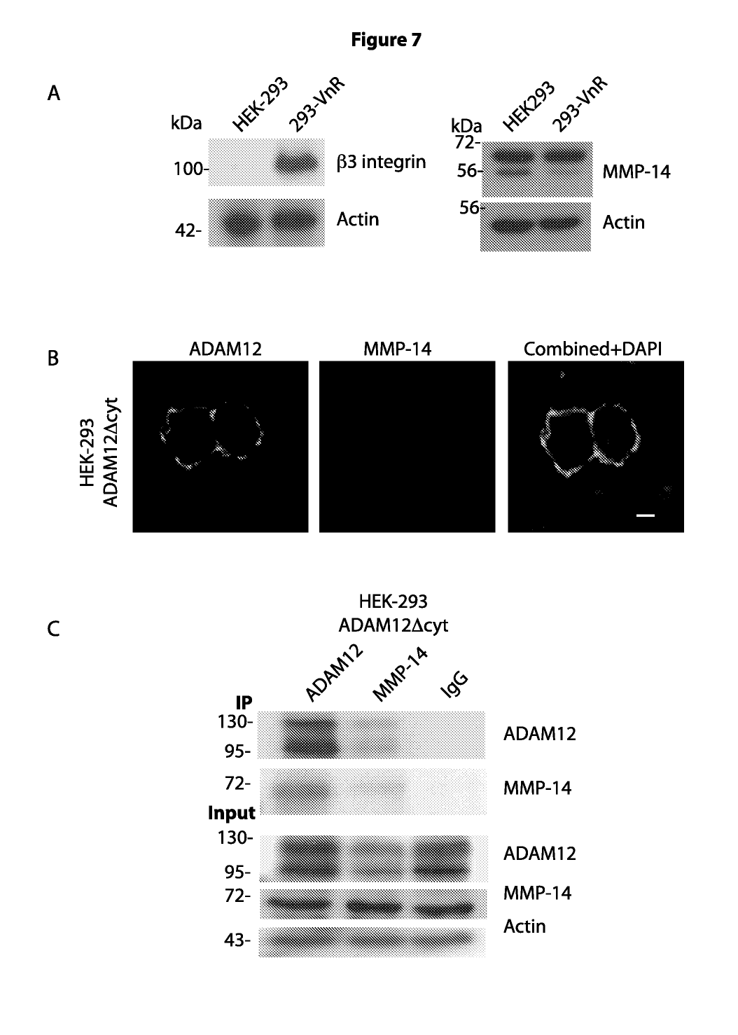

FIG. 7. Expression of β3 Integrin in 293-Vnr Direct Endogenous MMP-14 to the Cell Surface.

(A) Western blot analysis of β3 integrin and MMP-14 from HEK.293 and 293-Vnr cells. Actin was used as loading control. (B) 2HEK-293 cells were transfected with either vector control or ADAM12Δcyt-GFP. Cells were permeabilized and stained for MMP-14, ADAM12 and the nucleus. Scale bar=20 μm. (C) HEK-293 were transfected with ADAM12Δcyt-GFP, immunoprecipitation mouse IgG or mAbs against ADAM12 (6E6) or MMP-14. Precipitates and input samples were analysed by Western blotting with the indicated antibodies.

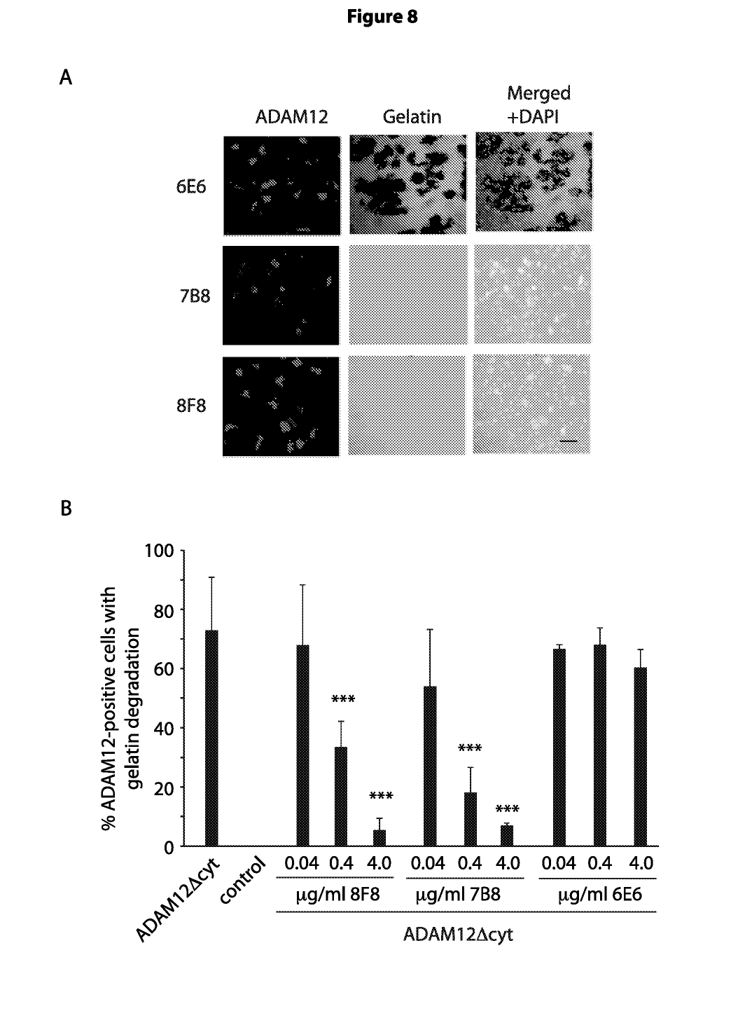

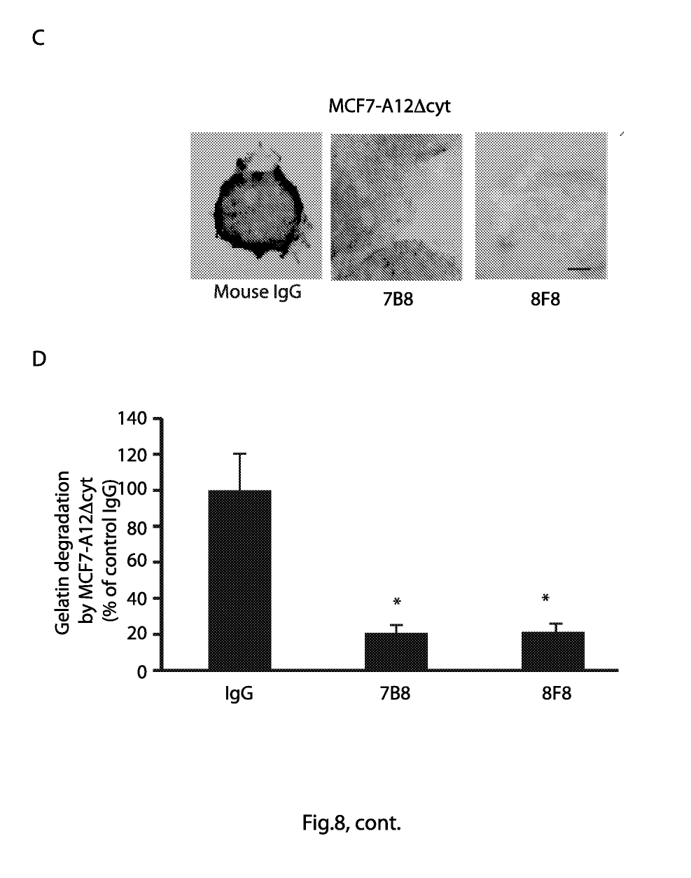

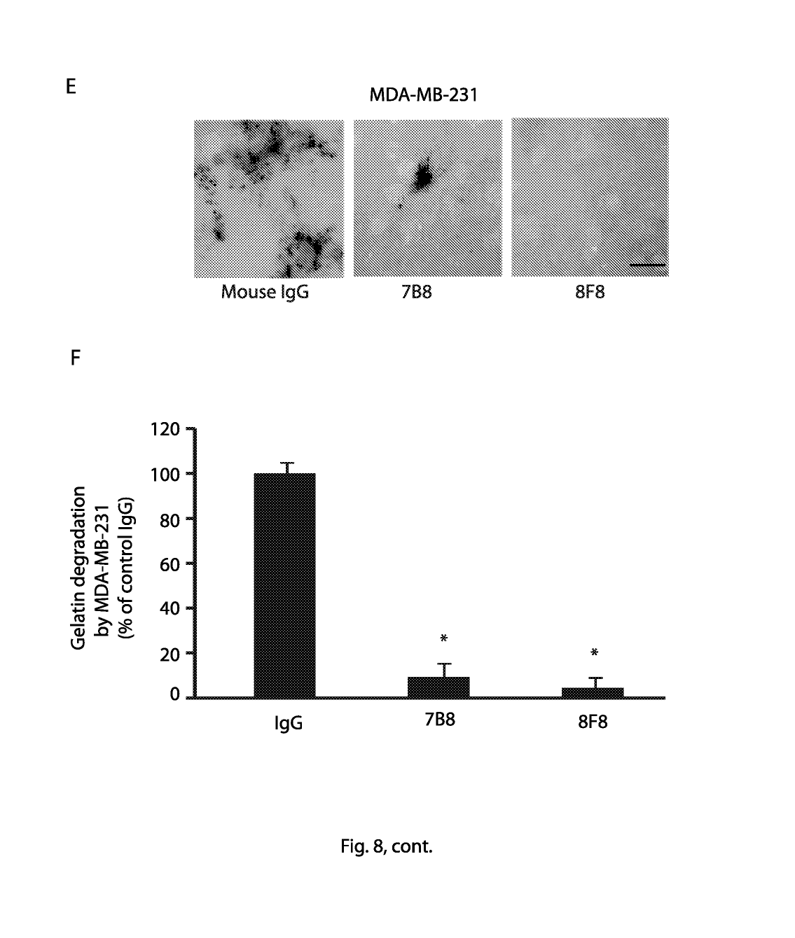

FIG. 8. Monoclonal Antibodies (mAbs) Against ADAM12 Inhibit ADAM12-Induced Gelatin Degradation.

(A) In situ solid-phase gelatinase assay of 293-VnR cells transfected with ADAM12Δcyt and treated overnight with 4 μg/ml of mAbs against ADAM12: 6E6, 7B8 and 8F8. Dishes were stained using the ADAM12 monoclonal antibody 6E6 and DAPI. Scale bar=45 μm. (B) In situ solid-phase gelatinase assay of 293-VnR cells transfected with ADAM12Δcyt (or control vector) and treated overnight with mAbs against ADAM12 in increasing concentration (0.04-4.0 μg/ml). Data represent the mean percentage (±s.d.) of ADAM12-positive cells with gelatin degradation. (C-F) In situ solid-phase gelatinase assay of MCF7-A12Δcyt (C,D) and MDA-MB-231 (E,F) cells treated overnight with control mouse IgG or mAbs 7B8 and 8F8 (10 μg/ml). Scale bar: (C)=20 μm and (E)=10 μm. (D,F) Mean percentage of gelatin degradation (±s.d.) for MCF7-A12Δcyt (D) and MDA-MB-231 (F) cells treated with 7B8, 8F8, or control mouse IgG (10 μg/ml) relative to the mean control IgG degradation value (set at 100%). *P<0.05, **P<0.01, ***P<0.001, Anova (B) and Student's t-test (D,F).

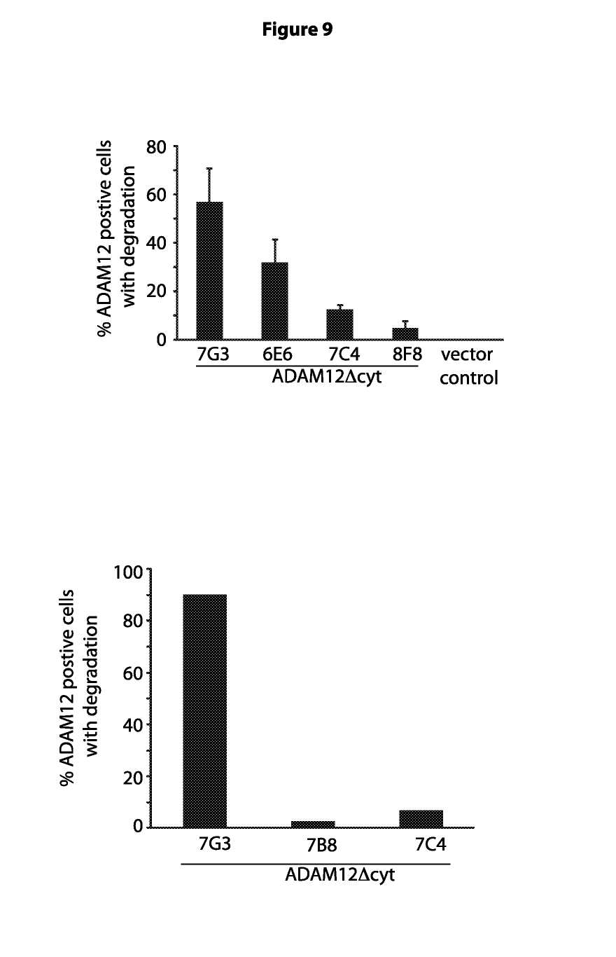

FIG. 9. Monoclonal Antibodies (mAbs) Against ADAM12 Inhibit ADAM12-Induced Gelatin Degradation.

Upper panel: In situ solid-phase gelatinase assay of 293-VnR cells transfected with ADAM12Δcyt and treated overnight with 4 μg/ml of mAbs against ADAM12: 7G3, 6E6, 7C4, and 8F8. Lower panel: In situ solid-phase gelatinase assay of 293-VnR cells transfected with ADAM12Δcyt and treated overnight with 4 μg/ml of mAbs against ADAM12: 7G3, 7C4, and 7B8.). Data represent the mean percentage (±s.d.) of ADAM12-positive cells with gelatin degradation.

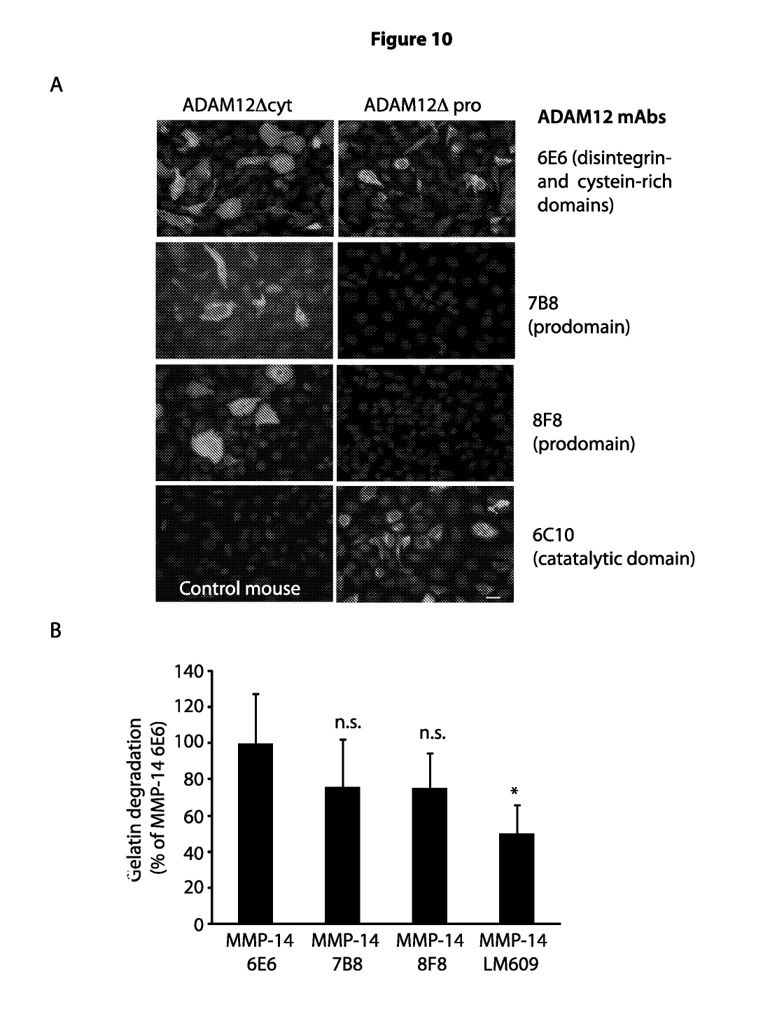

FIG. 10. 7B8 and 8F8 Recognise the Prodomain of ADAM12.

(A) 293-VnR cells were transfected with ADAM12Δcyt or ADAM12 lacking its prodomain (ADAM12pro). The transfected cells were fixed in cold methanol for 10 min, washed in PBS, and immunostained with 6E6, 7B8, 8F8, or 6C10 ADAM12 antibodies or control mouse IgG DAP. Scale bar=25 μm. (B) Gelatin degradation in 293-VnR cells transfected with MMP-14. Antibodies 6E6, 7B8, and 8F8 against ADAM12 and LM609 against αVβ3 integrin were added to the cultures (10 μg/ml) overnight. Data are expressed as the mean percentage of gelatin degradation (±s.d.) relative to the mean MMP-14-6E6 antibody degradation value (set at 100%); n=3. *P<0.001, Student's t-test.

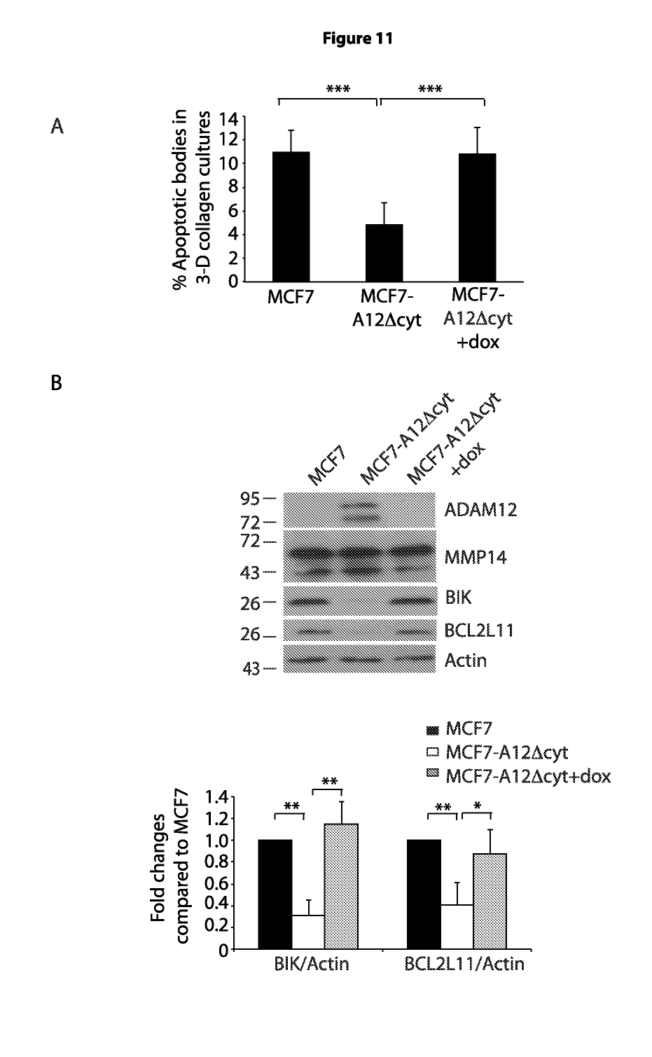

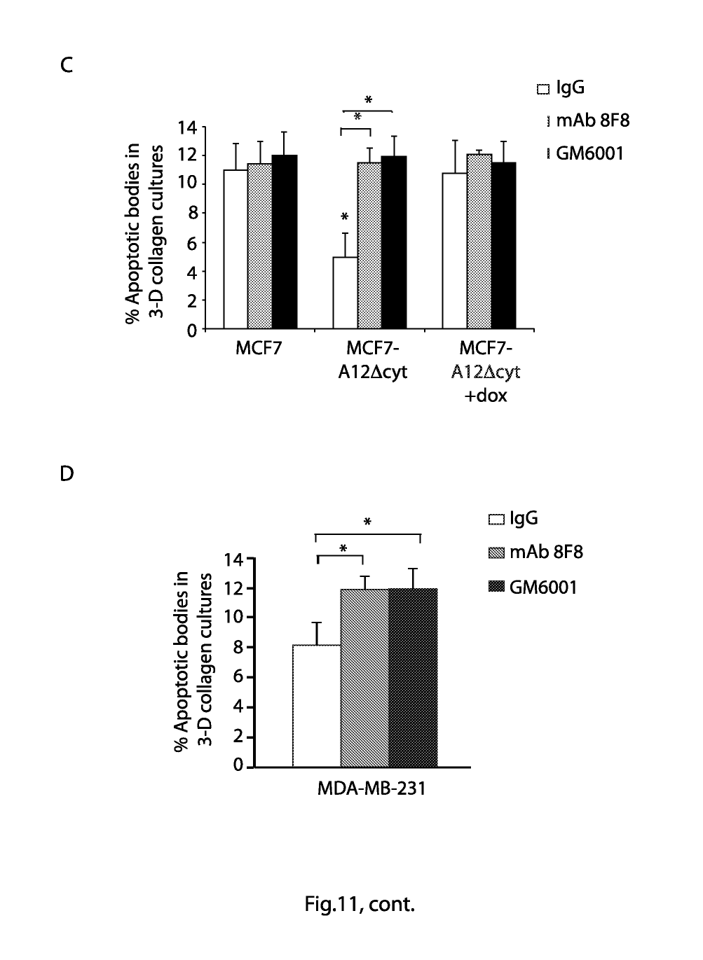

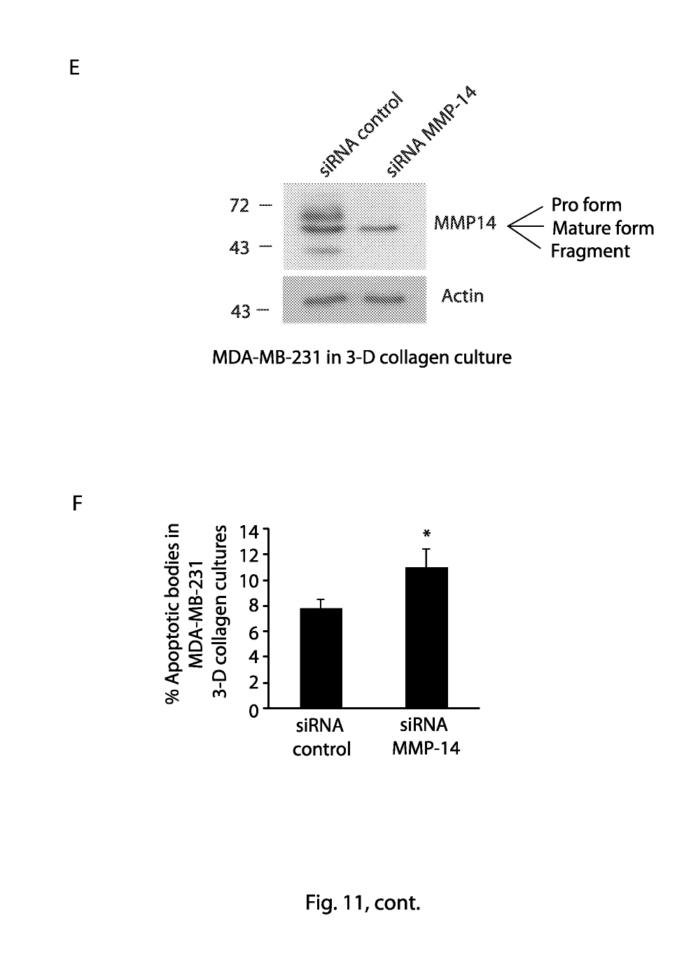

FIG. 11. ADAM12 Protects Tumour Cells Against Apoptosis In Vitro.

(A) MCF7, MCF7-A12Δcyt, and MCF7-A12Δcyt+dox cell lines were embedded in 3-D collagen and the mean percentage of apoptotic bodies (±s.d.) was determined from >500 cells per experiment. (B) Western blot analysis of ADAM12, MMP-14, BIK, and BIM from cells recovered from 3-D collagen. The bar graph depicts levels of BIK and BIM; determined by quantification of the band intensities (using ImageJ software) and normalized to actin. The MCF7 level was set to 1. (C,D) Mean percentage of apoptotic bodies (±s.d.) from >500 cells of 3-D collagen cultures of MCF7, MCF7-A12Δcyt, and MCF7-A12Δcyt+dox (C) and MDA-MB-231. (D) Cells treated every second day with control mouse IgG, 10 μg/ml 8F8, or 10 μM GM6001. (E) Western blot of cells recovered from 3-D cultures of MDA-MB-231 cells analyzed for MMP-14 after siRNA treatment. (F) Mean percentage of apoptotic bodies (±s.d.) from more than 500 cells of 4-day 3-D collagen cultures of MDA-MB-231 cells treated with MMP-14 siRNA or control siRNA prior to growth in collagen gels. *P<0.05, **P<0.01, ***P<0.001, Student's t-test.

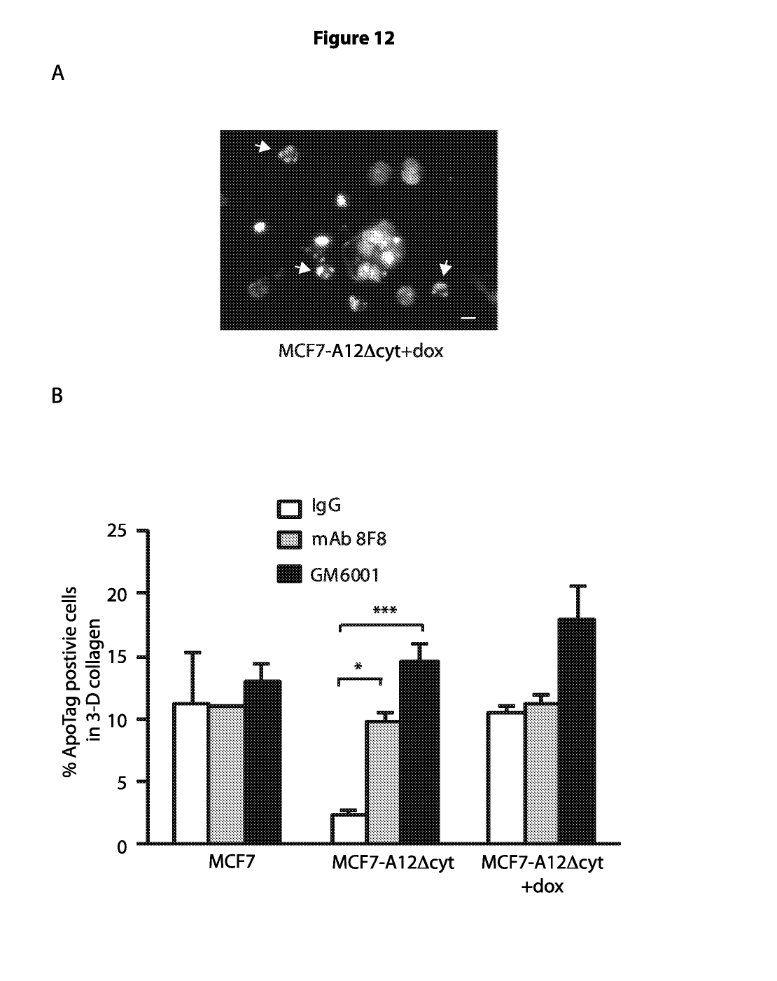

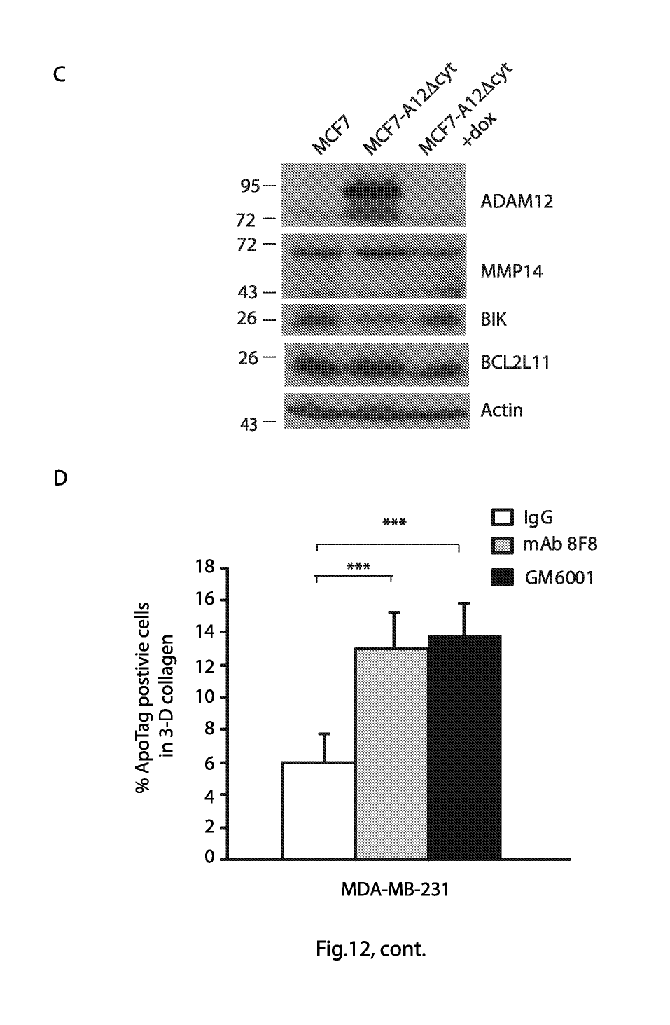

FIG. 12. ADAM12 Protects Tumour Cells Against Apoptosis In Vitro.

(A) A representative micrograph of MCF7-A12Δcyt+dox cells extracted from 3-D cultures and stained with DAPI. Apoptosis was evaluated by counting the percentage of the number of cells with chromatin condensation and nuclear fragmentation (arrow=apoptotic bodies). Scale bar=8 μm. (B) Mean percentage of ApopTag positive cells (±s.d.) from >500 cells of 3-D collagen cultures of MCF7, MCF7-A12Δcyt, and MCF7-A12Δcyt+dox cells treated every second day with control mouse IgG, 10 μg/ml 8F8, or 10 μM GM6001. (C) Western blot analysis of ADAM12, MMP-14, BIK, and BCL2L11 from cells grown in 2-D culture plates. (D) Mean percentage of ApopTag positive cells (±s.d.) from >500 cells of 3-D collagen cultures of MDA-MB-231 cells treated every second day with control mouse IgG, 10 μg/ml 8F8, or 10 μM GM6001. *P<0.05, ***P<0.001.

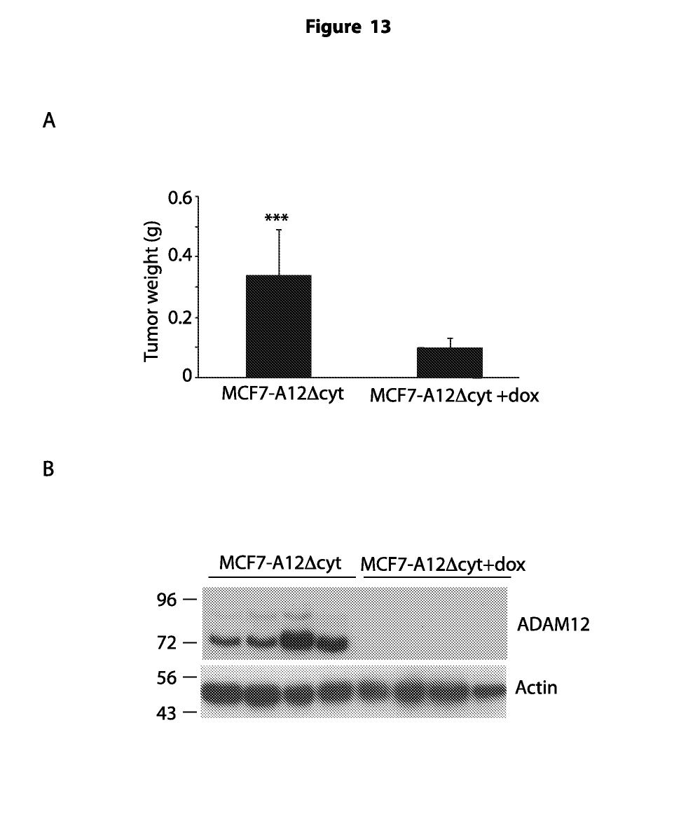

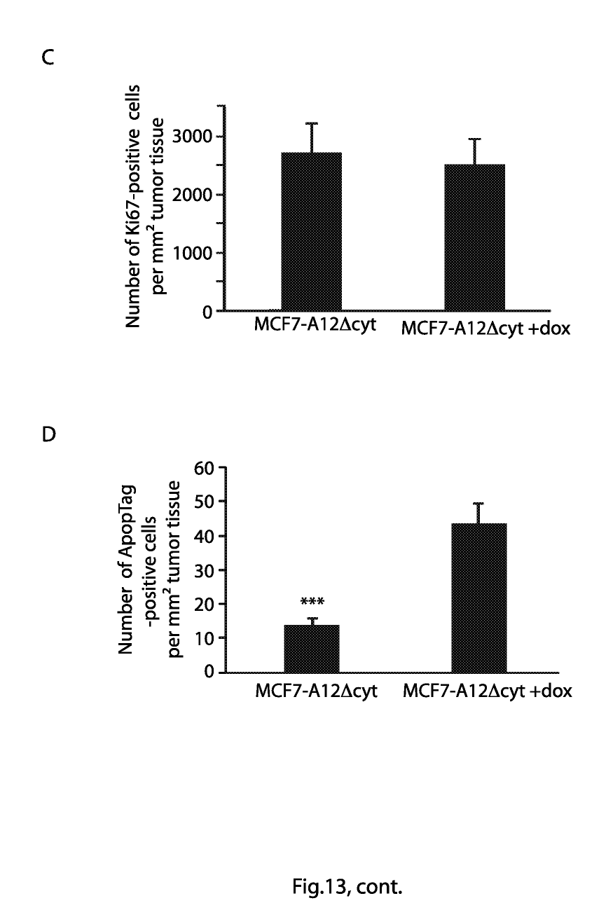

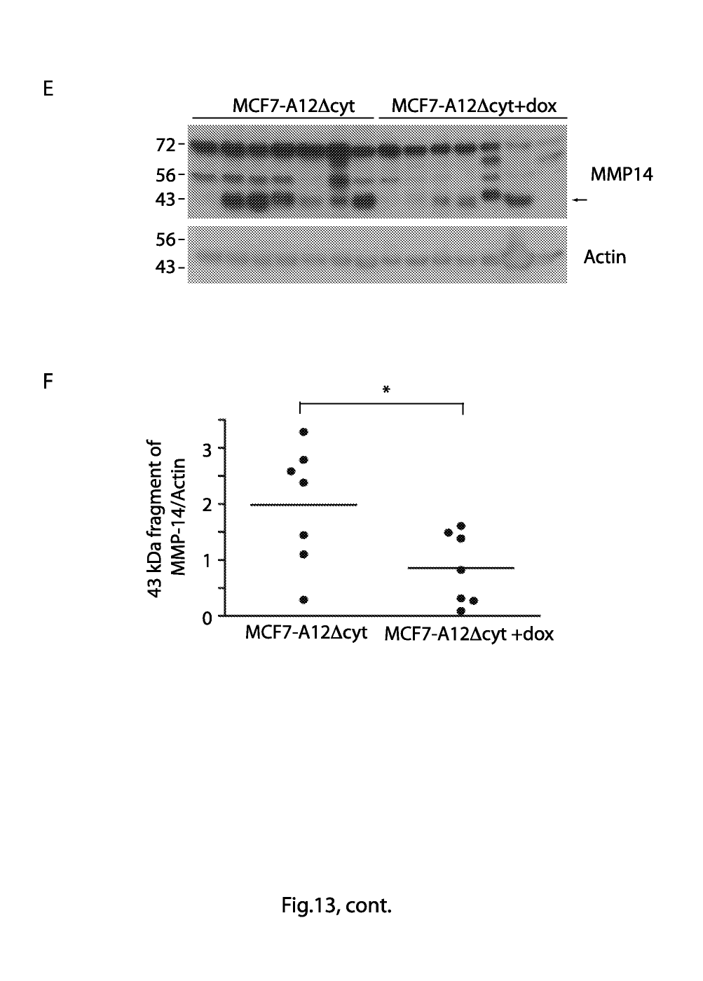

FIG. 13. ADAM12 Accelerates Tumour Growth and Inhibits Tumour Apoptosis in Vivo.

MCF7-A12Δcyt tumour cells were orthotopically implanted in the mammary glands of female mice. Some mice received doxycycline in their drinking water (MCF7-A12Δcyt+dox tumours, n=8), whereas other mice (MCF7-A12Δcyt tumours, n=8) did not. (A). Data represent mean tumour mass (±s.d.). (B) Western blot analysis of tumour extracts for ADAM12. (C) Mean cell proliferation (±s.d.) was calculated using Metamorf software program for nuclei counting of images from 5 areas of each MCF7-A12Δcyt and MCF7-A12Δcyt+dox tumour tissue immunostained for Ki67 staining. (D) Similar counting method as (C) were used to estimate mean number of ApopTag-positive cells (±s.d.) in tumour tissue. (E) Western blot analysis of MCF7-A12Δcyt and MCF7-A12Δcyt+dox tumour extracts for MMP-14. (F) Graphical representation of the levels of the 43 kDa fragment of MMP-14 (arrow in E); determined by quantification of the Western blot band intensities (using ImageJ software) and normalized to actin. *P<0.05, **P<0.01, ***P<0.001, Student's t-test.

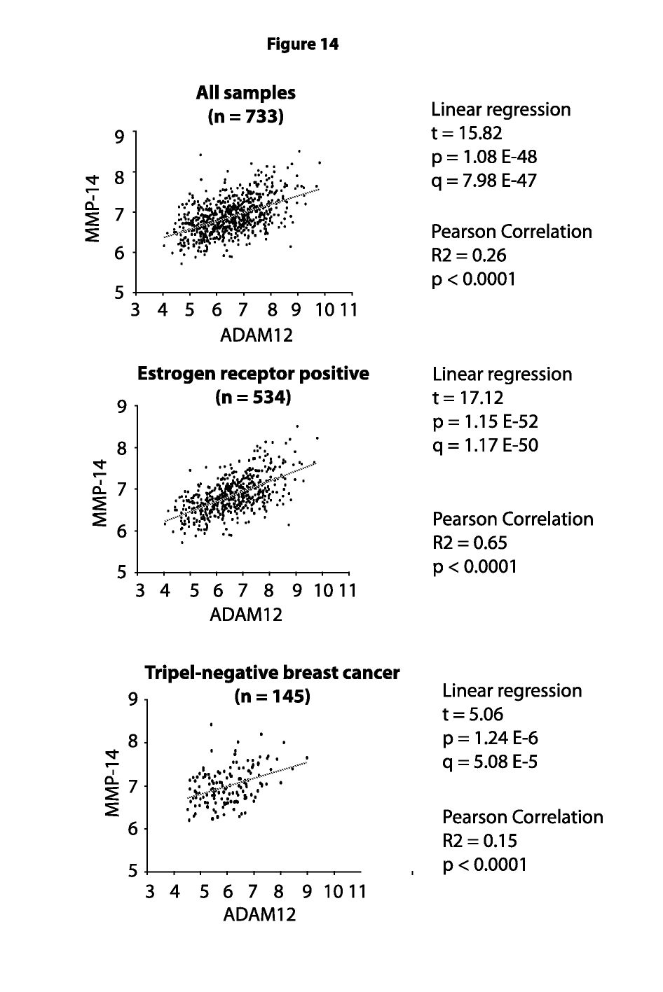

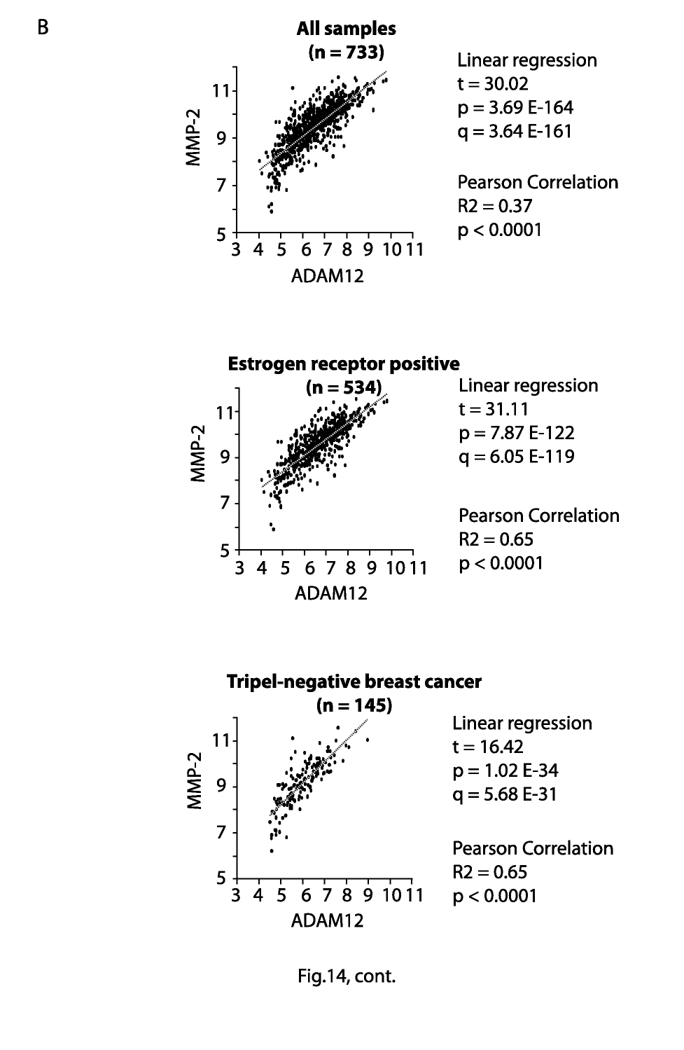

FIG. 14. Positive Correlation Between Expression of ADAM12 and Expression of MMP-14 and MMP-2.

Correlation analysis of ADAM12-L, MMP-14, and MMP-2 gene expression in 733 human breast tumours from 4 different cohorts: EMC286; Erasmus; TRANSBIG; and Mainz. Estrogen receptor-positive tumours (n=534) and triple-negative breast cancers (n=145) were also assessed by gene-expression profile. (A) Correlation between ADAM12 and MMP-14. (B) Correlation between ADAM12 and MMP-2. A simple linear regression analysis and Pearson correlation were performed.

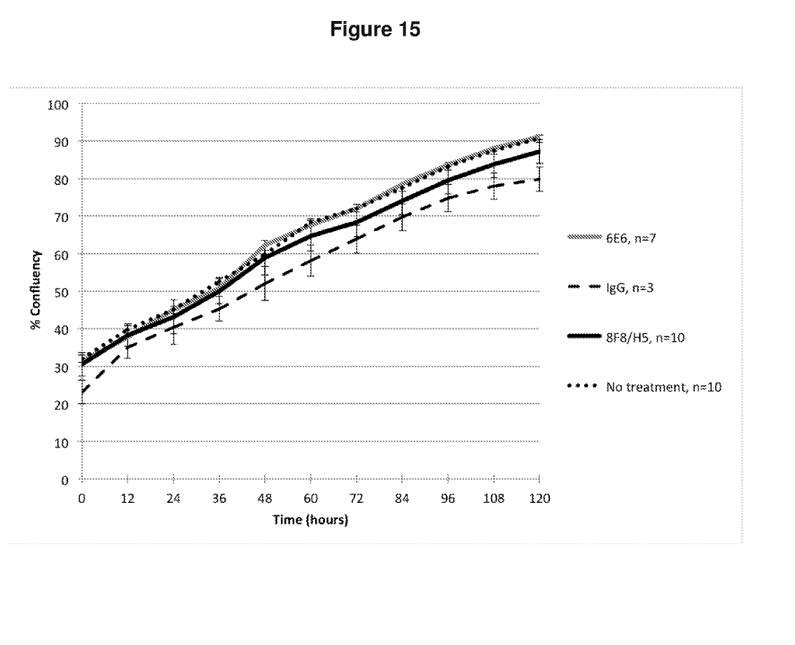

FIG. 15. No Effect of 8F8 on Tumour Cell Growth In Vitro.

The proliferation of MCF7-A12Δcyt cells was measured as % confluency by IncuCyte™ technology (Essen BioScience). Cells were seeded in 24-well plates (10⁵ cells/well), left untreated or treated every 24 h with 10 μg/ml 8F8 or 6E6 against ADAM12 or corresponding amounts of control mouse IgG for a total of 5 days. The graph shows the percentage confluency of the cell culture as a function of time. Data are shown as the average+1-SEM of at least 3 independent experiments (individual n values are shown on the graph), each performed in triplicates. ANOVA showed no statistically significant differences between groups, and simple linear regression analysis revealed similar growth rates for all culture conditions.

### DETAILED DESCRIPTION OF THE INVENTION

### Definitions

### Antibodies

The term ‘antibody’ describes a functional component of serum and is often referred to either as a collection of molecules (antibodies or immunoglobulin) or as one molecule (the antibody molecule or immunoglobulin molecule). An antibody molecule is capable of binding to or reacting with a specific antigenic determinant (the antigen or the antigenic epitope), which in turn may lead to induction of immunological effector mechanisms. An individual antibody molecule is usually regarded as monospecific, and a composition of antibody molecules may be monoclonal (i.e., consisting of identical antibody molecules) or polyclonal (i.e., consisting of different antibody molecules reacting with the same or different epitopes on the same antigen or on distinct, different antigens). Each antibody molecule has a unique structure that enables it to bind specifically to its corresponding antigen, and all natural antibody molecules have the same overall basic structure of two identical light chains and two identical heavy chains. Antibodies are also known collectively as immunoglobulins. The terms antibody or antibodies as used herein is used in the broadest sense and covers intact antibodies, chimeric, humanized, fully human and single chain antibodies, as well as binding fragments of antibodies, such as Fab, F(ab′)₂, Fv fragments or scFv fragments, as well as multimeric forms such as dimeric IgA molecules or pentavalent IgM.

### Naturally Occurring Antibody

The term ‘naturally occurring antibody’ refers to heterotetrameric glycoproteins capable of recognising and binding an antigen and comprising two identical heavy (H) chains and two identical light (L) chains inter-connected by disulfide bonds. Each heavy chain comprises a heavy chain variable region (abbreviated herein as V_{H}) and a heavy chain constant region (abbreviated herein as C_{H}). Each light chain comprises a light chain variable region (abbreviated herein as V_{L}) and a light chain constant region (abbreviated herein as C_{L}). The V_{H} and V_{L} regions can be further subdivided into regions of hypervariability, termed complementarity determining regions (CDRs), interspersed with regions that are more conserved, termed framework regions (FRs). Antibodies may comprise several identical heterotetramers.

### Antigen

An antigen is a molecule comprising at least one epitope. The antigen may for example be a polypeptide, polysaccharide, protein, lipoprotein or glycoprotein.

### Epitope

An epitope is a determinant capable of specific binding to an antibody. Epitopes may for example be comprised within polypeptides, polysaccharide, proteins, lipoproteins or glycoproteins. Epitopes usually consist of chemically active surface groupings of molecules such as amino acids or sugar side chains and usually have specific three dimensional structural characteristics, as well as specific charge characteristics. Epitopes may be conformational or nonconformational, wherein binding to the former but not the latter is lost in the presence of denaturing solvents. Epitopes may be continuous or discontinuous, wherein a discontinuous epitope is a conformational epitope on a protein antigen which is formed from at least two separate regions in the primary sequence of the protein.

### Antibodies

The invention relates to an antibody capable of specifically binding an epitope within the prodomain of ADAM12 (SEQ ID NO: 2), said antibody selected from the group consisting of:
i the monoclonal antibody produced by the hybridoma cell line 7B8 deposited under the Budapest Treaty with HPA Cultures collection (ECACC) under accession number 13082101;
ii the monoclonal antibody produced by the hybridoma cell line 8F8 deposited under the Budapest Treaty with HPA Cultures collection (ECACC) under accession number 13082102;
iii an antibody capable of binding the same epitope as 7B8 or 8F8;
iv an antibody capable of inhibiting binding of 7B8 or 8F8 to ADAM12;
v an antibody having the V_{H} and V_{L} of 7B8 or 8F8;
vi an antibody having the CDRs of the V_{H} and V_{L} of 7B8 or 8F8.

In another aspect the invention relates to the antibody defined herein above for use as a medicament.

In another aspect the invention relates to an antibody or a functional equivalent thereof, capable of specifically recognising and binding an epitope within the prodomain of ADAM12 (SEQ ID NO: 2), wherein said antibody or functional equivalent thereof specifically recognises at least part of an epitope recognised by one or more reference antibodies selected from the group consisting of:
i the monoclonal antibody produced by the hybridoma cell line 7B8 deposited under the Budapest Treaty with HPA Cultures collection (ECACC) under accession number 13082101;
ii the monoclonal antibody produced by the hybridoma cell line 8F8 deposited under the Budapest Treaty with HPA Cultures collection (ECACC) under accession number 13082102;
   for use in a method of treatment of cancer in a subject.

In some embodiments, the invention relates to an antibody or a functional equivalent thereof, capable of specifically recognising and binding an epitope within the prodomain of ADAM12 (SEQ ID NO: 2), wherein said antibody or functional equivalent thereof specifically recognises at least part of an epitope recognised by one or more reference antibodies selected from the group consisting of:
i the monoclonal antibody produced by the hybridoma cell line 7B8 deposited under the Budapest Treaty with HPA Cultures collection (ECACC) under accession number 13082101;
ii the monoclonal antibody produced by the hybridoma cell line 8F8 deposited under the Budapest Treaty with HPA Cultures collection (ECACC) under accession number 13082102; and
iii the monoclonal antibody produced by the hybridoma cell line 8F10 deposited under the Budapest Treaty with HPA Cultures collection (ECACC) under accession number 13082103;
for use in a method of treatment of cancer in a subject.

The antibody according to the present invention may be any polypeptide or protein capable of recognising and binding an antigen. Preferably, said antibody is capable of specifically binding said antigen. By the term “specifically binding” is meant binding with at least 10 times higher affinity to the antigen than to a non-specific antigen (e.g. BSA).

Preferably said antibody is a naturally occurring antibody or a functional equivalent thereof. A naturally occurring antibody is a heterotetrameric glycoprotein capable of recognising and binding an antigen comprising two identical heavy (H) chains and two identical light (L) chains inter-connected by disulfide bonds. Each heavy chain comprises or preferably consists of a heavy chain variable region (abbreviated herein as V_{H}) and a heavy chain constant region (abbreviated herein as C_{H}). Each light chain comprises or preferably consists of a light chain variable region (abbreviated herein as V_{L}) and a light chain constant region (abbreviated herein as C_{L}). The V_{H} and V_{L} regions can be further subdivided into regions of hypervariability, termed complementarity determining regions (CDRs), interspersed with regions that are more conserved, termed framework regions (FRs). Each V_{H} and V_{L} comprises and preferably consists of three CDRs and four FRs, arranged from amino-terminus to carboxy-terminus in the following order: FR1, CDR1, FR2, CDR2, FR3, CDR3, FR4.

The naturally occurring antibody may also be a heavy-chain antibody (HCAbs) as produced by camelids (camels, dromedaries and llamas). HCAbs are homodimers of heavy chains only, devoid of light chains and the first constant domain (Hamers-Casterman et al., 1993). Other naturally occurring antibodies may be devoid of light chains as is the case for the New or Nurse Shark Antigen Receptor (NAR) protein, which exists as a dimer of two heavy chains with no associated light chains. Each chain is composed of one variable (V) and five constant domains. The NAR proteins constitute a single immunoglobulin variable-like domain (Greenberg et al., 1995.) which is much lighter than an antibody molecule.

Naturally occurring antibodies according to the invention may consist of one heterotetramer or they may comprise several identical heterotetramers. Thus, the naturally occurring antibody according to the invention may for example be selected from the group consisting of IgG, IgM, IgA, IgD and IgE. The subunit structures and three-dimensional configurations of these different classes of immunoglobulins are well known. In a preferred embodiment of the invention the antibody is IgG, e.g. IgG-1, IgG-2, IgG-3 and IgG-4.

Naturally occurring antibodies according to the invention may be antibodies of a particular species, for example the antibody may be a murine, a rat, a rabbit, a goat, a sheep, a chicken, a donkey, a camelid or a human antibody. The antibody may be a murine monoclonal antibody. The antibody according to the invention may however also be a hybrid between antibodies from several species, for example the antibody may be a chimeric antibody, such as a humanised antibody. Human and humanised antibodies are discussed in further detail herein below.

The antibody according to the invention may be a monoclonal antibody, such as a naturally occurring monoclonal antibody or it may be polyclonal antibodies, such as naturally occurring polyclonal antibodies. Preferably, the antibodies are monoclonal. Monoclonal and polyclonal antibodies are discussed in further detail herein below.

### Antigen Binding Fragments of Antibodies

Antigen binding fragments of antibodies are fragments of antibodies retaining the ability to specifically bind to an antigen. Thus in some embodiments the functional equivalent of an antibody is a binding fragment of an antibody. Preferably, said fragment is an antigen binding fragment of a naturally occurring antibody. It has been shown that the antigen-binding function of an antibody can be performed by fragments of a full-length antibody. Examples of antigen binding fragments of naturally occurring antibodies include for example (i) a Fab fragment, a monovalent fragment consisting of the V_{L}, V_{H}, C_{L} and C_{H1} domains; (ii) a F(ab′)₂ fragment, a bivalent fragment comprising two Fab fragments linked by a disulfide bridge at the hinge region; (iii) an Fd fragment consisting of the V_{H} and C_{H1} domains; (iv) an Fv fragment consisting of the V_{L} and V_{H} domains of a single arm of an antibody or (v) a dAb fragment (VVard et al., 1989), which consists of a V_{H} domain. Fab fragments may be prepared by papain digestion. F(ab′)₂ fragments may be prepared by pepsin treatment.

The antigen binding fragment of an antibody preferably comprises at least one complementarity determining region (CDR) or more preferably a combination of two or more isolated CDRs which may optionally be joined by a synthetic linker. A further example of an antigen binding fragment of an antibody is binding-domain immunoglobulin fusion proteins comprising (i) an antigen binding site fused to an immunoglobulin hinge region polypeptide, (ii) an immunoglobulin heavy chain CH2 constant region fused to the hinge region, and (iii) an immunoglobulin heavy chain CH3 constant region fused to the CH2 constant region. The antigen binding site can be a heavy chain variable region or a light chain variable region. Such binding-domain immunoglobulin fusion proteins are further disclosed in US 2003/0118592 and US 2003/0133939. These antibody fragments are obtained using conventional techniques known to those with skill in the art, and the fragments are screened for utility in the same manner as are intact antibodies.

The antigen binding fragment of an antibody may also be a diabody, which are small antibody fragments with two antigen-binding sites. Diabodies preferably comprises a heavy chain variable domain (VH) connected to a light chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., 1993.

### Heterospecific Antibodies

The antibody according to the invention may also be a “heterospecific antibody”, such as a bispecific antibody. A bispecific antibody is a protein or polypeptide, which comprises two different antigen binding sites with different specificities. For example, the bispecific antibody may recognise and bind to (a) a first epitope within a first antigen and (b) a second epitope within a second antigen; or it may recognise and bind to two different epitopes within the same antigen. The term “heterospecific antibody” is intended to include any protein or polypeptide, which has more than two different antigen binding sites with different specificities. For example, the heterospecific antibody may recognise and bind to (a) a first epitope on a first antigen, (b) a second epitope on a second antigen and (c) a third epitope on a third antigen; or it may recognise and bind to (a) a first epitope on a first antigen and (b) a second and third epitope on a second antigen; or it may recognise and bind to different epitopes on the same antigen. Accordingly, the invention includes, but is not limited to, bispecific, trispecific, tetraspecific, and other multispecific antibodies which are directed to different epitopes on the same or on different antigens.

Bispecific antibodies may for example be prepared starting from monoclonal antibodies, for example by fusing two hybridoma's in order to combine their specificity, by chemical crosslinking or using recombinant technologies. For example the V_{H} and V_{L} of two different antibodies (1 and 2) may be linked by recombinant means to form “cross-over” chains V_{H}1-V_{L}2 and V_{H}2-V_{L}1, and then dimerised to reassemble both antigen-binding sites (see WO 94/09131). Bispecific antibodies may also be prepared by genetically linking two single chain antibodies with different specificities as for example described in WO 94/13806. Also two antigen binding fragments of an antibody may be linked.

### Human and Humanised Antibodies

It is not always desirable to use non-human antibodies for human therapy, accordingly the antibody according to the invention may be a human antibody or a humanised antibody.

A human antibody as understood herein is an antibody, which is obtained from a system using human immunoglobulin sequences. Human antibodies may for example be antibodies isolated from an animal (e.g., a mouse) that is transgenic or transchromosomal for human immunoglobulin genes or a hybridoma prepared therefrom. Human antibodies may also be isolated from a host cell transformed to express the antibody, e.g., from a transfectoma. Human antibodies may also be isolated from a recombinant, combinatorial human antibody library.

Human antibodies have variable and constant regions derived from human germline immunoglobulin sequences. In certain embodiments, however, such recombinant human antibodies can be subjected to in vitro mutagenesis or in vivo somatic mutagenesis and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

A human antibody is preferably at least 90%, more preferably at least 95%, even more preferably at least 96%, 97%, 98%, or 99% identical in amino acid sequence to the amino acid sequence encoded by a wild type human immunoglobulin gene.

Said transgenic or transchromosomal animal may contain a human immunoglobulin gene minilocus that encodes unrearranged human heavy (p and/or γ) and κ light chain immunoglobulin sequences. Furthermore, the animal may contain one or more mutations that inactivate the endogenous heavy and light chain loci. Examples of such animals are described in Lonberg et al. (1994) and WO 02/43478.

The antibody according to the invention may be a chimeric antibody, i.e. an antibody comprising regions derived from different species. The chimeric antibody may for example comprise variable regions from one animal species and constant regions from another animal species. For example, a chimeric antibody can be an antibody having variable regions which derive from a mouse monoclonal antibody and constant regions which are human. Such antibodies may also be referred to as humanised antibodies. Thus, the antibody according to the invention may also be a humanised antibody, which is encoded partly by sequences obtained from human germline immunoglobulin sequences and partly from other sequences. Said other sequences are preferably germline immunoglobulines from other species, more preferably from other mammalian species. In particular a humanised antibody may be an antibody in which the antigen binding site is derived from an immunoglobulin from a non-human species, preferably from a non-human mammal, e.g. from a mouse or a rat, whereas some or all of the remaining immunoglobulin-derived parts of the molecule are derived from a human immunoglobulin. The antigen binding site from said non-human species may for example consist of a complete V_{L} or V_{H} or both, or one or more CDRs grafted onto appropriate human framework regions in V_{L} or V_{H} or both. Thus, in a humanized antibody, the CDRs can be from a mouse or rat monoclonal antibody and the other regions of the antibody are of human origin.

### Monoclonal Antibodies

Monoclonal antibodies (MAbs) refer to a population of antibodies, wherein the antibody molecules are similar and thus recognise and bind to the same epitope. Monoclonal antibodies are in general produced by a host cell line and frequently by a hybridoma cell line. Methods of making monoclonal antibodies and antibody-synthesizing hybridoma cells are well known to those skilled in the art. Antibody producing hybridomas may for example be prepared by fusion of an antibody producing B lymphocytes with an immortalized B-lymphocyte cell line. Monoclonal antibodies according to the present invention may for example be prepared by the standard somatic cell hybridization technique of Kohler and Milstein, Nature 256:495 (1975) or as described in Antibodies: A Laboratory Manual, By Ed Harlow and David Lane, Cold Spring Harbor Laboratory Press, 1988. Said monoclonal antibodies may be derived from any suitable mammalian species, however frequently the monoclonal antibodies will be rodent antibodies for example murine or rat monoclonal antibodies.

### Polyclonal Antibodies

Polyclonal antibodies refer to a population of antibodies comprising a mixture of different antibody molecules recognising and binding to a specific given antigen, hence polyclonal antibodies may recognise different epitopes within said antigen. In general polyclonal antibodies are purified from the serum of an animal, preferably a mammal, which previously has been immunized with the antigen. Polyclonal antibodies may for example be prepared by any of the methods described in Antibodies: A Laboratory Manual, By Ed Harlow and David Lane, Cold Spring Harbor Laboratory Press, 1988.

### Recombinant Antibodies

The antibody according to the present invention may also be a recombinant antibody, i.e. an antibody prepared, expressed, created or isolated by recombinant means. Recombinant antibodies according to the invention may be for example be produced using a synthetic library or by phage display. In some embodiments, the antibody is produced in a recombinant cell. The recombinant cell may be a microorganism selected from the group comprising bacteria and eukaryotic microorganisms. Recombinant antibodies may be produced in microbial host organisms, such as bacteria, yeasts or cultures of cells derived from multicellular organisms. Frequently, Escherichia coli is useful as host organism. Frequently recombinant antibodies are fragments of naturally occurring antibodies comprising at least one antigen binding site, such as a Fab fragment, a F(ab′)₂, a Fv fragment or the recombinant antibody is a scFV. Thus in some embodiments the antibody is a fragment of a naturally occurring antibody comprising at least one antigen binding site, such as a Fab fragment, a F(ab′)₂, a Fv fragment. In other embodiments, the recombinant antibody is a scFV.

Recombinant antibodies may be identified using various systems, such as phage display or ribosome display. The starting point of phage display is usually a library of antibodies, such as single chain antibodies or fragments of naturally occurring antibodies expressed by a phage. Various different kinds of phages are suitable for use in phage display, e.g. M13, fd filamentous phage, T4, T7 or λ phage. Phagemids may also be used, but that usually requires use of a helper phage. Typically the library comprises in the range of 10⁷ to 10¹⁵, such as 10⁹ to 10¹¹ different phages. The antibodies may be either of naive or immune origin. The antibodies of the library may be fused to a phage coat protein (e.g. g3p or g8p) in order to ensure display on the surface. Thus, the antibody (fragment) may be encoded by a nucleic acid sequence, which is cloned upstream or downstream of a nucleic acid encoding a phage coat protein, which is operably linked to a suitable promoter.

The genomic information coding for antibody e.g. for the antibody variable domains may be obtained from B cells of non-immunised or immunised donors using recombinant DNA technology to amplify the V_{H} and V_{L} gene segments and cloning into an appropriate phage. Synthetic libraries may be prepared by rearranging V_{H} and V_{L} gene segments in vitro and/or by introducing artificial sequences into V_{H} and V_{L} gene segments. For example synthetic libraries may be prepared using a V_{H} and V_{L} gene framework, but introducing into this artificial complementarity determining regions (CDRs), which may be encoded by random oligonucleotides. The library may also be different libraries, which are then combined in the host cell. Thus, one library may comprise heavy chain sequences, such as the heavy chain Fv fragment or Fab fragment or V_{H} and the other light chain sequences, such as the light chain Fv fragment or Fab fragment V_{L}. Typically several rounds of selection, e.g. 2 to 5, such as 2 to 3 are performed. This may be done by immobilising the antigen, contacting the antigen with the phage and isolating the bound phages. The antigen may be immobilised on any suitable solid surface, such as a plastic surface, beads (such as magnetic beads), a resin in a column, or it may be expressed on the surface of a cell.

### Single Chain Antibodies

Naturally occurring antibodies are heterotetramers. However, the antibody according to the present invention may also be a single polypeptide comprising one or more antigen binding sites. Such antibodies are also referred to as “single chain antibodies”. Thus the antibody according to the present invention may also be a single chain antibody. Single chain antibodies may comprise the two domains of the Fv fragment, V_{L} and V_{H}. To obtain such single chain antibodies the genes encoding the V_{L} and V_{H} may be joined, using recombinant methods. Usually they are separated by a synthetic linker, for example a linker of 5 to 100, such as of 5 to 50, for example of 25 amino acids. Said linker may either connect the N-terminus of the V_{H} with the C-terminus of the V_{L}, or vice versa. This enables production of a single protein chain in which the V_{L} and V_{H} regions pair to form monovalent antibody like molecules (also known as single chain antibodies or single chain Fv (scFv); see e.g., Bird et al. (1988) Science 242:423-426; and Huston et al. (1988) Proc. Natl. Acad. Sci. USA 85:5879-5883).

The single chain antibody may also be a divalent antibody, e.g. a single peptide chain comprising two V_{H} and two V_{L} regions, which may be linked two by two by a linker. The single chain antibody may also be a multivalent antibody, e.g. a single peptide chain comprising multiple V_{H} and multiple V_{L} regions, which may be linked two by two by a linker. The V_{H} and V_{L} regions may be identical or different, yielding monospecific or heterospecific antibodies, respectively. Said V_{H} and said V_{L} may be a naturally occurring V_{H} or V_{L}, or a synthetic V_{H} and V_{L} comprising at least one antigen binding site. Preferably said V_{H} and V_{L} are naturally occurring V_{H} and V_{L}.

### ADAM12 and Prodomain

Two naturally occurring human ADAM12 splice variants exist, which were named ADAM12-L (SEQ ID NO: 1 shows the amino acid sequence, SEQ ID NO: 3 shows the mRNA sequence for human ADAM12) and ADAM12-S. The domain composition of ADAM12-L resembles the prototypical transmembrane ADAM protein. ADAM12L is a 909 amino acid long protein, comprising a signal peptide (amino acids 1 to 28), the prodomain (amino acids 29-206 of SEQ ID NO:1, shown in SEQ ID NO: 2), a metalloprotease (amino acids 207-417), a disintegrin domain (amino acids 418-512), a cysteine-rich domain (amino acids 513-588), a fusion-like domain (amino acids 589-614), an EGF-like domain (amino acids 615-708), a transmembrane domain (amino acids 709-729) and a cystoplasmic tail (amino acids 730-909). ADAM12L further contains 5 N-glycosylation domains, of which two are found within the prodomain (NYT, amino acids 111-113, and NES, amino acids 149-151). ADAM12-S, the soluble splice variant, contains the same domains as ADAM12-L, but lacks the transmembrane domain, and the cytoplasmic tail is replaced by a stretch of 33 amino acid in the C-terminus.

Through the disintegrin and cysteine-rich domains, the ADAM12 molecule is involved in cell adhesion via binding to integrins and syndecan, respectively (Iba et al., 1999; Thodeti et al., 2005a; Thodeti et al., 2005b). In particular, the inventors and others have shown that the disintegrin domain of ADAM12-S interacts with αVβ3 integrin, which in turn is known to interact with MMP-14. The inventors also found that MMP-14 and ADAM12 are likely to interact directly (FIG. 6C). The prodomain of ADAM12, approximately 26 kDa, is thought to primarily keep the ADAM12 molecule in an inactive form until cleavage at the furin-site between the prodomain and the catalytic domain. After furin cleavage the prodomain remains associated with the mature molecule through non-covalent bonds.

### Antibodies Binding to the Prodomain of ADAM12

Thus it is a goal of the invention to provide an antibody or functional equivalent thereof specifically recognising and binding an epitope within the prodomain of ADAM12, wherein said antibody or functional equivalent thereof specifically recognises at least part of an epitope recognised by one or more reference antibodies selected from the group consisting of:
i the monoclonal antibody produced by the hybridoma cell line 7B8 deposited under the Budapest Treaty with HPA Cultures collection (ECACC) under accession number 13082101;
ii the monoclonal antibody produced by the hybridoma cell line 8F8 deposited under the Budapest Treaty with HPA Cultures collection (ECACC) under accession number 13082102.

It is a further goal of the invention to provide an antibody or functional equivalent thereof specifically recognising and binding an epitope within the prodomain of ADAM12, wherein said antibody or functional equivalent thereof specifically recognises at least part of an epitope recognised by one or more reference antibodies selected from the group consisting of:
i the monoclonal antibody produced by the hybridoma cell line 7B8 deposited under the Budapest Treaty with HPA Cultures collection (ECACC) under accession number 13082101;
ii the monoclonal antibody produced by the hybridoma cell line 8F8 deposited under the Budapest Treaty with HPA Cultures collection (ECACC) under accession number 13082102.

In some embodiments is provided an antibody or functional equivalent thereof specifically recognising and binding an epitope within the prodomain of ADAM12, wherein said antibody or functional equivalent thereof specifically recognises at least part of an epitope recognised by one or more reference antibodies selected from the group consisting of:
i the monoclonal antibody produced by the hybridoma cell line 7B8 deposited under the Budapest Treaty with HPA Cultures collection (ECACC) under accession number 13082101;
ii the monoclonal antibody produced by the hybridoma cell line 8F8 deposited under the Budapest Treaty with HPA Cultures collection (ECACC) under accession number 13082102;
iii the monoclonal antibody produced by the hybridoma cell line 8F10 deposited under the Budapest Treaty with HPA Cultures collection (ECACC) under accession number 13082103.

It is a further object of the invention to provide such antibodies selected from the group consisting of 7B8, 8F8, 8F10 and 7C4.

The antibody may be a monoclonal or polyclonal antibody. The antibody may originate from any organism known in the art to be suitable for the production of antibodies, such as live organisms, for example mice, sheep, rabbit, or cell cultures such as bacterial cells, yeast cells, plant cells, insect cells or mammalian cell lines such as CHO cells.

The antibody may be humanised. Preferably the antibody is a murine monoclonal antibody.

Also within the scope of the invention are functional equivalents which comprise a binding fragment of an antibody. The fragment may be selected from the group consisting of Fab, Fab′, F(ab′)₂ and Fv fragments such as ScFv fragments. The functional equivalent may also be a single chain antibody. Other embodiments relate to functional equivalents that e.g. comprise or consist of the binding fragment of an antibody binding to the prodomain of ADAM12. In some embodiments the functional equivalent may further comprise engineered domains which may for example increase the half-life, the stability, the bioavailability, the solubility or other relevant characteristics of the antibody fragment. For example, it is known in the art that engineered intradomain sulphide bonds can stabilise antibodies (Wozniak-Knopp et al. 2012). Other methods of stabilisation include e.g. the use of a peptide linker between the V_{H} and the V_{L} domains, resulting in stabilisation of Fv fragments (Reiter et al., 1994).

The functional equivalent may also be an antibody mimetic, or a small molecule mimicking an antibody. Antibody mimetics are organic compounds, including, but not limited to, nucleic acids, that can specifically bind antigens, but that are not structurally related to antibodies. They are usually artificial peptides or proteins, typically with a molar mass of about 3 to 20 kDa. Some types have an antibody-like β-sheet structure. Antibody mimetics sometimes display better solubility, tissue penetration, stability towards heat and enzymes compared to antibodies, as well as comparatively low production costs.

The antibody of the invention is capable of binding to the prodomain of ADAM12. Also within the scope of the invention are antibodies or functional equivalents thereof which are capable of binding to an epitope within the prodomain of ADAM12, said epitope comprising amino acid residues 1 to 10 of SEQ ID NO: 2, or amino acid residues 11 to 20 of SEQ ID NO: 2, or amino acid residues 21 to 30 of SEQ ID NO: 2, or amino acid residues 31 to 40 of SEQ ID NO: 2, or amino acid residues 41 to 50 of SEQ ID NO: 2, or amino acid residues 51 to 60 of SEQ ID NO: 2, or amino acid residues 61 to 70 of SEQ ID NO: 2, or amino acid residues 71 to 80 of SEQ ID NO: 2, or amino acid residues 81 to 90 of SEQ ID NO: 2, or amino acid residues 91 to 100 of SEQ ID NO: 2, or amino acid residues 101 to 110 of SEQ ID NO: 2, or amino acid residues 111 to 120 of SEQ ID NO: 2, or amino acid residues 121 to 130 of SEQ ID NO: 2, or amino acid residues 131 to 140 of SEQ ID NO: 2, or amino acid residues 141 to 150 of SEQ ID NO: 2, or amino acid residues 151 to 160 of SEQ ID NO: 2, or amino acid residues 161 to 170 of SEQ ID NO: 2, or amino acid residues 171 to 178 of SEQ ID NO: 2.

The antibody of the invention may comprise zero, one or two of the glycosylation domains of the prodomain of ADAM12. Thus the antibody may comprise amino acids residues 111-113 (NYT) of SEQ ID NO: 1, and/or amino acid residues 149-151 (NES) of SEQ ID NO: 1, or none of these amino acid residues.

The antibody may be any protein or polypeptide containing an antigen binding site, such as a single polypeptide, a protein or a glycoprotein. Preferably, the antigen binding site comprises at least one CDR, or more preferably a variable region.

Thus the antigen binding site may comprise a V_{H} and/or a V_{L}. In an antibody, the V_{H} and V_{L} together may contain the antigen binding site, however, either one of the V_{H} or the V_{L} may comprise an antigen binding site. In particular, the CDRs may identify the specificity of the antibody and accordingly it is preferred that the antigen binding site comprises one or more CDRs, preferably at least 1, more preferably at least 2, yet more preferably at least 3, even more preferably at least 4, yet more preferably at least 5, even more preferably 6 CDRs. It is preferable that the antigen binding site comprises at least one CDR3, more preferably at least the CDR3 of the heavy chain.

The antibody may for example be an antigen binding fragment of an antibody, preferably an antigen binding fragment of a naturally occurring antibody, a heterospecific antibody, a single chain antibody or a recombinant antibody.

An antibody according to the invention may comprise one or more antigen binding sites. Naturally occurring heterotetrameric antibodies comprise two antigen binding sites.

In one embodiment of the present invention the antibody is an antibody comprising one or more of the following specific heavy chain CDRs. Preferably, the antibody comprises at least one, more preferably at least two, even more preferably all three of the following CDRs: CDR1, CDR2 and/or CDR3 of the heavy chain of an antibody binding to the prodomain of ADAM12 as described above; and the antibody preferably comprises at least one, preferably two, even more preferably all three of the following CDRs: CDR1, CDR2 and/or CDR3 of the light chain of an antibody binding to the prodomain of ADAM12 as described above.

In some embodiments the antibody is constructed by domain shuffling. Some antibodies according to the invention comprise, for the light chain:
i. CDR1 from any of the light chain of an antibody binding to the prodomain of ADAM12;
ii. CDR2 from any of the light chain of an antibody binding to the prodomain of ADAM12;
iii. CDR3 from any of the light chain of an antibody binding to the prodomain of ADAM12;
   and, for the heavy chain:
i. CDR1 from any of the heavy chain of an antibody binding to the prodomain of ADAM12;
ii. CDR2 from any of the heavy chain of an antibody binding to the prodomain of ADAM12;
iii. CDR3 from any of the heavy chain of an antibody binding to the prodomain of ADAM12.

The antibody may further comprise one or more FRs selected from group consisting of:
i. FR1, FR2, FR3 or FR4 of the heavy chain of an antibody binding to the prodomain of ADAM12; and
ii. FR1, FR2, FR3 or FR4 of the light chain of an antibody binding to the prodomain of ADAM12.

Specific embodiments thus relate to antibodies comprising one or more of the heavy chain CDRs, one or more of the light chain CDRs and one or more of the FRs of 7B8, 8F8, 8F10 and 7C4.

The antibodies of the invention may be obtained by domain shuffling. Domain shuffling can be performed by methods known in the art, such as by traditional cloning or by ligase-independent methods, e.g. uracil-specific excision reagent (USER) cloning and fusion (Nour-Eldin et al., 2010; Villiers et al., 2010) or gap repair (Eckert-Boulet et al., 2012).

Alternatively, the antibody may comprise functional equivalents of at least one, more preferably at least two, even more preferably all three of said heavy chain CDRs. Preferably, said functional equivalents are identical to said heavy chain CDRs except for one to two, more preferably except for one substitution or deletion or insertion.

Alternatively, the antibody may comprise functional equivalents of at least one, more preferably at least two, even more preferably all three of said light chain CDRs. Preferably, said functional equivalents are identical to said light chain CDRs expect for one to two, more preferably except for one substitution or deletion or insertion.

In a preferred embodiment the antibody or functional equivalent thereof comprises at least three, yet more preferably at least four, even more preferably at least five, yet more preferably all six of the above-mentioned CDRs.

In some embodiments the antibody is an antibody variant. Such variants include, but are not limited to, antibodies which have been modified in order to increase half-life, solubility and/or bioavailability.

### Binding to Prodomain of ADAM12

The antibody or functional equivalent thereof is capable of binding to the prodomain of ADAM12 or to an epitope within the prodomain of ADAM12. In order to determine whether an antibody or a functional equivalent thereof is capable of binding to the prodomain of ADAM12 or to an epitope within the prodomain of ADAM12, methods known in the art can be used, such as, but not limited to, immunofluorescence-based methods optionally combined with Western blotting using cell lysates from cells transfected with the prodomain of ADAM12. Truncated versions of the prodomain of ADAM12 may also be used for transfecting cells, if it is desirable to obtain information about which epitope of the prodomain the antibody is capable of binding to.

### Inhibition of Gelatin Degradation

In some embodiments, the antibody or functional equivalent thereof is capable of inhibiting gelatin degradation. In order to determine whether an antibody or a functional equivalent thereof is capable of inhibiting gelatin degradation, assays known in the art may be used. For example, cells treated with an antibody of the invention may be seeded on a dish coated with gelatin and gelatin degradation can be assessed over time. Other methods suitable for testing inhibition of gelatin degradation will be recognised by the skilled person.

### Inhibition of Catalytic Activity of ADAM12

In some embodiments, the antibody or functional equivalent thereof does not inhibit the catalytic activity of ADAM12. Methods for determining whether the antibody or functional equivalent thereof inhibits the catalytic activity of ADAM12 are known in the art. For example, in vitro quenched-fluorescent peptide cleavage assay or cell-based ectodomain shedding assay may be used. Other methods suitable for testing inhibition of the catalytic activity of ADAM12 will be recognised by the skilled person.

### Inhibition of MMP-14-Induced Increase of BIK

In some embodiments, the antibody or functional equivalent thereof inhibits MMP-14-induced increase of BIK. Methods for determining whether the antibody or functional equivalent thereof inhibits MMP-14-induced increase of BIK are known in the art. For example, levels of BIK may be determined by Western blotting on cell lysates. Other methods suitable for testing inhibition of MMP-14-induced increase of BIK will be recognised by the skilled person.

### Induction of Apoptosis

In some embodiments, the antibody or functional equivalent thereof induces apoptosis. Methods for determining whether the antibody or functional equivalent thereof induces apoptosis are known in the art. For example, kits for determining apoptotic activity which are commercially available may be used, or the fraction of cells with apoptotic bodies may be determined visually. Other methods suitable for testing inhibition of MMP-14-induced increase of BIK will be recognised by the skilled person.

### Method for Treating Cancer

Another purpose of the invention is to provide a method for treating cancer comprising the step of administering a therapeutically effective dosage of the antibody capable of binding to the prodomain of ADAM12 as defined herein to a subject in need thereof. Preferably, the antibody is selected from the group consisting of 7B8, 8F8, 8F10 and 7C4 and functional equivalents or variants thereof, as defined above.

By ‘subject in need thereof’ is understood a subject in need of a treatment against cancer, such as a subject suffering from cancer for the first time, or a subject suffering from a recurrent cancer. The subject is an animal, preferably a mammal, such as, but not limited to, a human, a dog, a cat, a horse.

The cancer to be treated may be selected from the group comprising: cancer of the breast, bladder, ovary, colon, uterus, cervix, kidney, prostate, oesophagus, renal cells, pancreas, rectum, stomach, squamous cells, lung, head and neck, skin, testicles, liver, oral cavity, brain, bone, bone marrow and blood cells. Thus the cancer to be treated may be selected from the group consisting of cancer of the breast, bladder, colon, liver, lung, oral cavity, stomach, brain and bone. In a preferred embodiment, the cancer to be treated is a bladder cancer. In another preferred embodiment, the cancer to be treated is a breast cancer. In other preferred embodiments the cancer to be treated is characterised by elevated levels of ADAM12. The levels of ADAM12 may be determined in vitro or in vivo, by measuring the levels of mRNA or of protein. For example, the levels of the ADAM12 protein may be determined by Western blot, by immunostaining, or by other methods known in the art (see for example US2009/0029372). The levels of mRNA may be determined by Northern blot, RT-PCR, microarray analysis or by other methods known in the art (see US2009/0029372).

In some embodiments, the antibody to be administered for treating cancer is capable of inhibiting the formation of a complex between ADAM12, MMP-14 and αVβ3. For example, the antibody may inhibit complex formation by inhibiting the interaction between ADAM12 and MMP-14, or between ADAM12 and αVβ3, or between MMP-14 and αVβ3. In particular, the antibody is capable of inhibiting recruitment of MMP-14 by ADAM12. Thus in some embodiments the antibody is capable of inhibiting recruitment of MMP-14 to the cell surface. Thus in some embodiments the antibody is capable of inhibiting gelatin degradation. Preferably, the antibody to be administered in the present method does not inhibit the catalytic activity of ADAM12. In some embodiments, the antibody may additionally inhibit MMP-14-induced increase of Bcl2-interacting killer (BIK) protein. In preferred embodiments, the antibody is capable of inducing apoptosis. In particular, apoptosis of tumour cells is induced by the antibody. In preferred embodiments, the antibody does not affect cellular growth. In other embodiments, the antibody is stable in the serum. Preferably, the antibody is not toxic to the host organism after administration. In particular, 8F8 does not affect cellular growth as measured in vitro (FIG. 15). 8F8 and 7B8 were found to be stable in mouse serum. 8F8 and 7B8 show no sign of toxicity after injection in immune compromised mice.

It is within the scope of the present invention to provide a medicament comprising the antibody as defined above as an active ingredient. In some embodiments the invention relates to the use of said medicament for treating a cancer.

Also within the scope of the invention is the use of the antibody as defined above for the preparation of a medicament for treating cancer in a subject in need thereof. In some embodiments the antibody is selected from the group comprising 7B8, 8F8, 8F10 and 7C4. The antibody may be a functional equivalent or a variant of an antibody, as described above.

Thus the invention relates to a method for treatment of a cancer in an individual in need thereof, the method comprising the steps of:
a. providing a sample from tumour tissue of an individual,
b. determining the expression level of ADAM12 in the sample of step a,
c. correlating the expression level of step b with the expression level of a control tissue,
d. assessing a treatment regime,
e. administering to the individual a therapeutically effective amount of an antibody, a functional equivalent thereof or a variant thereof as defined above.

Also disclosed is a method of treatment of cancer in an individual in need thereof, the method comprising the steps of:
a. providing a sample from tumour tissue of an individual,
b. determining the degradation level of gelatin in the sample of step a,
c. correlating the expression level of step b with the expression level of a control tissue,
d. assessing a treatment regime,
e. administering to the individual a therapeutically effective amount of an antibody, a functional equivalent thereof or a variant thereof as defined above.

The invention further relates to a method of treatment of cancer in an individual in need thereof, said method comprising administering an antibody which inhibits gelatin degradation.

The invention also relates to a method of treatment of cancer in an individual in need thereof, said method comprising administering an antibody directed against the prodomain of ADAM12.

### Pharmaceutical Composition and Administration Forms

The present invention also encompasses pharmaceutical compositions comprising the antibody, functional equivalent or variant thereof as defined herein. In the present context, the term ‘antibody’ and ‘compound’ will be used as synonyms when discussing pharmaceutical composition and administration forms.

In the present context, the term “a pharmaceutical composition” as used herein typically means a composition containing an antibody of the present invention, a variant or functional equivalent thereof, and optionally one or more pharmaceutically acceptable carriers or excipients, and may be prepared by conventional techniques, e.g. as described in Remington: The Science and Practice of Pharmacy 1995, edited by E. W. Martin, Mack Publishing Company, 19th edition, Easton, Pa. The compositions may appear in conventional forms, for example capsules, tablets, aerosols, solutions, suspensions or topical applications. Typically, the pharmaceutical compositions of the present invention may be formulated for parenteral administration e.g., by intravenous or subcutaneous injection, and may be presented in unit dose form in ampoules, pre-filled syringes, small volume infusion or in multi-dose containers with an added preservative. The compositions may take such forms as suspensions, solutions, or emulsions in oily or aqueous vehicles, for example solutions in aqueous polyethylene glycol. The compositions may be suitable for oral ingestion. Examples of oily or nonaqueous carriers, diluents, solvents or vehicles include propylene glycol, polyethylene glycol, vegetable oils (e.g., olive oil), and injectable organic esters (e.g., ethyl oleate), and may contain formulatory agents such as preserving, wetting, emulsifying or suspending, stabilizing and/or dispersing agents. Alternatively, the active ingredient may be in powder form, obtained by aseptic isolation of sterile solid or by lyophilisation from solution for constitution before use with a suitable vehicle, e.g., sterile, pyrogen-free water. Oils useful in parenteral formulations include petroleum, animal, vegetable, or synthetic oils. Specific examples of oils useful in such formulations include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters. The parenteral formulations typically will contain from about 0.0001 to about 25%, such as from about 0.5 to about 25%, by weight of the active ingredient in solution. Preservatives and buffers may be used. In order to minimise or eliminate irritation at the site of injection, such compositions may contain one or more nonionic surfactants having a hydrophile-lipophile balance (HLB) of from about 12 to about 17. The quantity of surfactant in such formulations will typically range from about 0.000001 to about 15% by weight, such as from about 0.000001 to about 5% by weight or from about 5 to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol. The parenteral formulations can be presented in unit-dose or multi-dose sealed containers, such as ampoules and vials, and can be stored in a freeze-dried (lyophilized) condition requiring only the addition of the sterile liquid excipient, for example, water, for injections, immediately prior to use.

The main route of drug delivery according to this invention is however parenteral in order to introduce the agent into the blood stream to ultimately target the relevant tissue.

The agent may also be administered to cross any mucosal membrane of an animal to which the biologically active substance is to be given, e.g. in the nose, vagina, eye, mouth, genital tract, lungs, gastrointestinal tract, or rectum, preferably the mucosa of the nose, or mouth.

In a preferred embodiment the agent of the invention is administered parenterally, that is by intravenous, intramuscular, intraspinal, subcutaneous, intranasal, intrarectal, intravaginal or intraperitoneal administration. The subcutaneous and intramuscular forms of parenteral administration are generally preferred. Appropriate dosage forms for such administration may be prepared by conventional techniques. The compounds may also be administered by inhalation, which is by intranasal and oral inhalation administration. Appropriate dosage forms for such administration, such as an aerosol formulation or a metered dose inhaler, may be prepared by conventional techniques.

In one embodiment the pharmaceutical composition according to the present invention is formulated for parenteral administration such as by injection.

In a further embodiment the pharmaceutical composition according to the present invention is formulated for intravenous, intramuscular, intraspinal, intraperitoneal, subcutaneous, a bolus or a continuous administration.

The rate and frequency of the administration may be determined by the physician from a case to case basis. In one embodiment the administration occurs at intervals of 30 minutes to 24 hours, such as at intervals of 1 to 6 hours, such as three times a day.

The duration of the treatment may vary depending on severity of the condition. In one embodiment the duration of the treatment is from 6 to 72 hours. In chronic cases the duration of the treatment may be lifelong.

The dosage can be determined by the physician in charge based on the characteristics of the patient and the means and mode of administration. In one embodiment of the present invention, the dosage of the active ingredient of the pharmaceutical composition as defined herein above, is between 10 μg to 500 mg per kg body mass, such as between 20 μg and 400 mg, e.g. between 30 μg and 300 mg, such as between such as between 50 μg to 250 mg per kg body mass, such as between 40 μg and 200 mg, e.g. between 50 μg and 100 mg, such as between 60 μg and 90 μg, e.g. between 70 μg and 80 μg.

The dosage may be administered as a bolus administration or as a continuous administration. In relation to bolus administration the pharmaceutical composition may be administered at intervals of 30 minutes to 24 hours, such as at intervals of 1 to 6 hours. When the administration is continuous it is administered over an interval of time that normally is from 6 hours to 7 days. Preferably, the duration of the administration is from 24 hours to 7 days. The duration of the administration may be from 4 days to 150 days. In some embodiments the administration may be lifelong. However, normally the dosage will be administered as a bolus 1-3 times per day.

### Formulations

Whilst it is possible for the compounds of the present invention to be administered as the raw chemical, it is preferred to present them in the form of a pharmaceutical formulation. Accordingly, the present invention further provides a pharmaceutical formulation, for medicinal application, which comprises a compound of the present invention or a functional equivalent thereof, as herein defined, and a pharmaceutically acceptable carrier thereof.

In one embodiment the pharmaceutical composition as defined herein above comprises a pharmaceutically acceptable carrier.

The agents of the present invention may be formulated into a wide variety of dosage forms, suitable for the various administration forms discussed above.

The pharmaceutical compositions and dosage forms may comprise the antibody of the invention or its functional equivalent as the active component.

Furthermore, the pharmaceutical compositions may comprise pharmaceutically acceptable carriers that can be either solid or liquid.

Solid form preparations are normally provided for oral or enteral administration, such as powders, tablets, pills, capsules, cachets, suppositories, and dispersible granules. A solid carrier can be one or more substances which may also act as diluents, flavoring agents, solubilizers, lubricants, suspending agents, binders, preservatives, wetting agents, tablet disintegrating agents, or an encapsulating material.

Preferably, the composition will be about 0.5% to 75% by weight of a compound or compounds of the invention, with the remainder consisting of suitable pharmaceutical excipients. For oral administration, such excipients include pharmaceutical grades of mannitol, lactose, starch, magnesium stearate, sodium saccharine, talcum, cellulose, glucose, gelatin, sucrose, magnesium carbonate, and the like.

In powders, the carrier is a finely divided solid which is a mixture with the finely divided active component. In tablets, the active component is mixed with the carrier having the necessary binding capacity in suitable proportions and compacted in the shape and size desired. Powders and tablets preferably contain from one to about seventy percent of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as carrier providing a capsule in which the active component, with or without carriers, is surrounded by a carrier, which is in association with it. Similarly, cachets and lozenges are included. Tablets, powders, capsules, pills, cachets, and lozenges can be as solid forms suitable for oral administration.

Drops according to the present invention may comprise sterile or non-sterile aqueous or oil solutions or suspensions, and may be prepared by dissolving the active ingredient in a suitable aqueous solution, optionally including a bactericidal and/or fungicidal agent and/or any other suitable preservative, and optionally including a surface active agent. The resulting solution may then be clarified by filtration, transferred to a suitable container which is then sealed and sterilized by autoclaving or maintaining at 98-100° C. for half an hour. Alternatively, the solution may be sterilized by filtration and transferred to the container aseptically. Examples of bactericidal and fungicidal agents suitable for inclusion in the drops are phenylmercuric nitrate or acetate (0.002%), benzalkonium chloride (0.01%) and chlorhexidine acetate (0.01%). Suitable solvents for the preparation of an oily solution include glycerol, diluted alcohol and propylene glycol.

Also included are solid form preparations which are intended to be converted, shortly before use, to liquid form preparations for oral administration. Such liquid forms include solutions, suspensions, and emulsions. These preparations may contain, in addition to the active component, colorants, flavours, stabilizers, buffers, artificial and natural sweeteners, dispersants, thickeners, solubilizing agents, and the like.

Other forms suitable for oral administration or oral ingestion include liquid form preparations including emulsions, syrups, elixirs, aqueous solutions, aqueous suspensions, toothpaste, gel dentrifrice, chewing gum, or solid form preparations which are intended to be converted shortly before use to liquid form preparations. Emulsions may be prepared in solutions in aqueous propylene glycol solutions or may contain emulsifying agents such as lecithin, sorbitan monooleate, or acacia. Aqueous solutions can be prepared by dissolving the active component in water and adding suitable colorants, flavours, stabilizing and thickening agents. Aqueous suspensions can be prepared by dispersing the finely divided active component in water with viscous material, such as natural or synthetic gums, resins, methylcellulose, sodium carboxymethylcellulose, and other well known suspending agents. Solid form preparations include solutions, suspensions, and emulsions, and may contain, in addition to the active component, colorants, flavours, stabilizers, buffers, artificial and natural sweeteners, dispersants, thickeners, solubilizing agents, and the like.

The compounds of the present invention may be formulated for parenteral administration (e.g., by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, for example solutions in aqueous polyethylene glycol. Examples of oily or nonaqueous carriers, diluents, solvents or vehicles include propylene glycol, polyethylene glycol, vegetable oils (e.g., olive oil), and injectable organic esters (e.g., ethyl oleate), and may contain formulatory agents such as preserving, wetting, emulsifying or suspending, stabilizing and/or dispersing agents. Alternatively, the active ingredient may be in powder form, obtained by aseptic isolation of sterile solid or by lyophilisation from solution for constitution before use with a suitable vehicle, e.g., sterile, pyrogen-free water.

Oils useful in parenteral formulations include petroleum, animal, vegetable, or synthetic oils. Specific examples of oils useful in such formulations include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters.

Suitable soaps for use in parenteral formulations include fatty alkali metal, ammonium, and triethanolamine salts, and suitable detergents include (a) cationic detergents such as, for example, dimethyl dialkyl ammonium halides, and alkyl pyridinium halides; (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylenepolypropylene copolymers, (d) amphoteric detergents such as, for example, alkyl-.beta.-aminopropionates, and 2-alkyl-imidazoline quaternary ammonium salts, and (e) mixtures thereof.

The parenteral formulations typically will contain from about 0.5 to about 25% by weight of the active ingredient in solution. Preservatives and buffers may be used. In order to minimize or eliminate irritation at the site of injection, such compositions may contain one or more nonionic surfactants having a hydrophile-lipophile balance (HLB) of from about 12 to about 17. The quantity of surfactant in such formulations will typically range from about 5 to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol. The parenteral formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described.

The compounds of the invention can also be delivered topically for transdermal or transmucosal administration. Regions for topical administration include the skin surface and also mucous membrane tissues of the vagina, rectum, nose, mouth, and throat. Compositions for topical administration via the skin and mucous membranes should not give rise to signs of irritation, such as swelling or redness. Transdermal administration typically involves the delivery of a pharmaceutical agent for percutaneous passage of the drug into the systemic circulation of the patient. The skin sites include anatomic regions for transdermally administering the drug and include the forearm, abdomen, chest, back, buttock, mastoidal area, and the like.

The topical composition may include a pharmaceutically acceptable carrier adapted for topical administration. Thus, the composition may take the form of a suspension, solution, ointment, lotion, sexual lubricant, cream, foam, aerosol, spray, suppository, implant, inhalant, tablet, such as a sublingual tablet, capsule, dry powder, syrup, balm or lozenge, for example. Methods for preparing such compositions are well known in the pharmaceutical industry.

The compounds of the present invention may be formulated for topical administration to the epidermis as ointments, creams or lotions, or as a transdermal patch. Ointments and creams may, for example, be formulated with an aqueous or oily base with the addition of suitable thickening and/or gelling agents. Lotions may be formulated with an aqueous or oily base and will in general also containing one or more emulsifying agents, stabilizing agents, dispersing agents, suspending agents, thickening agents, or colouring agents. Formulations suitable for topical administration in the mouth include lozenges comprising active agents in a flavoured base, usually sucrose and acacia or tragacanth; pastilles comprising the active ingredient in an inert base such as gelatin and glycerin or sucrose and acacia; and mouthwashes comprising the active ingredient in a suitable liquid carrier.

Creams, ointments or pastes according to the present invention are semi-solid formulations of the active ingredient for external application. They may be made by mixing the active ingredient in finely-divided or powdered form, alone or in solution or suspension in an aqueous or non-aqueous fluid, with the aid of suitable machinery, with a greasy or non-greasy base. The base may comprise hydrocarbons such as hard, soft or liquid paraffin, glycerol, beeswax, a metallic soap; a mucilage; an oil of natural origin such as almond, corn, arachis, castor or olive oil; wool fat or its derivatives or a fatty acid such as steric or oleic acid together with an alcohol such as propylene glycol or a macrogel. The formulation may incorporate any suitable surface active agent such as an anionic, cationic or non-ionic surfactant such as a sorbitan ester or a polyoxyethylene derivative thereof. Suspending agents such as natural gums, cellulose derivatives or inorganic materials such as silicaceous silicas, and other ingredients such as lanolin, may also be included.

Lotions according to the present invention include those suitable for application to the skin or eye. An eye lotion may comprise a sterile aqueous solution optionally containing a bactericide and may be prepared by methods similar to those for the preparation of drops. Lotions or liniments for application to the skin may also include an agent to hasten drying and to cool the skin, such as an alcohol or acetone, and/or a moisturizer such as glycerol or an oil such as castor oil or arachis oil.

Transdermal delivery may be accomplished by exposing a source of the complex to a patient's skin for an extended period of time. Transdermal patches have the added advantage of providing controlled delivery of a pharmaceutical agent-chemical modifier complex to the body. See Transdermal Drug Delivery: Developmental Issues and Research Initiatives, Hadgraft and Guy (eds.), Marcel Dekker, Inc., (1989); Controlled Drug Delivery: Fundamentals and Applications, Robinson and Lee (eds.), Marcel Dekker Inc., (1987); and Transdermal Delivery of Drugs, Vols. 1-3, Kydonieus and Berner (eds.), CRC Press, (1987). Such dosage forms can be made by dissolving, dispersing, or otherwise incorporating the pharmaceutical agent-chemical modifier complex in a proper medium, such as an elastomeric matrix material. Absorption enhancers can also be used to increase the flux of the compound across the skin. The rate of such flux can be controlled by either providing a rate-controlling membrane or dispersing the compound in a polymer matrix or gel.

For example, a simple adhesive patch can be prepared from a backing material and an acrylate adhesive. The pharmaceutical agent-chemical modifier complex and any enhancer are formulated into the adhesive casting solution and allowed to mix thoroughly. The solution is cast directly onto the backing material and the casting solvent is evaporated in an oven, leaving an adhesive film. The release liner can be attached to complete the system.

Foam matrix patches are similar in design and components to the liquid reservoir system, except that the gelled pharmaceutical agent-chemical modifier solution is constrained in a thin foam layer, typically a polyurethane. This foam layer is situated between the backing and the membrane which have been heat sealed at the periphery of the patch.

For passive delivery systems, the rate of release is typically controlled by a membrane placed between the reservoir and the skin, by diffusion from a monolithic device, or by the skin itself serving as a rate-controlling barrier in the delivery system. See U.S. Pat. Nos. 4,816,258; 4,927,408; 4,904,475; 4,588,580, 4,788,062; and the like. The rate of drug delivery will be dependent, in part, upon the nature of the membrane. For example, the rate of drug delivery across membranes within the body is generally higher than across dermal barriers. The rate at which the complex is delivered from the device to the membrane is most advantageously controlled by the use of rate-limiting membranes which are placed between the reservoir and the skin. Assuming that the skin is sufficiently permeable to the complex (i.e., absorption through the skin is greater than the rate of passage through the membrane), the membrane will serve to control the dosage rate experienced by the patient.

Suitable permeable membrane materials may be selected based on the desired degree of permeability, the nature of the complex, and the mechanical considerations related to constructing the device. Exemplary permeable membrane materials include a wide variety of natural and synthetic polymers, such as polydimethylsiloxanes (silicone rubbers), ethylenevinylacetate copolymer (EVA), polyurethanes, polyurethane-polyether copolymers, polyethylenes, polyamides, polyvinylchlorides (PVC), polypropylenes, polycarbonates, polytetrafluoroethylenes (PTFE), cellulosic materials, e.g., cellulose triacetate and cellulose nitrate/acetate, and hydrogels, e.g., 2-hydroxyethylmethacrylate (HEMA).

The compounds of the present invention may also be formulated for administration as suppositories. A low melting wax, such as a mixture of fatty acid glycerides or cocoa butter is first melted and the active component is dispersed homogeneously, for example, by stirring. The molten homogeneous mixture is then poured into convenient sized molds, allowed to cool, and to solidify.

The active compound may be formulated into a suppository comprising, for example, about 0.5% to about 50% of a compound of the invention, disposed in a polyethylene glycol (PEG) carrier (e.g., PEG 1000 [96%] and PEG 4000 [4%].

The compounds of the present invention may be formulated for vaginal administration. Pessaries, tampons, creams, gels, pastes, foams or sprays containing in addition to the active ingredient such carriers as are known in the art to be appropriate.

The compounds of the present invention may be formulated for nasal administration. The solutions or suspensions are applied directly to the nasal cavity by conventional means, for example with a dropper, pipette or spray. The formulations may be provided in a single or multidose form. In the latter case of a dropper or pipette this may be achieved by the patient administering an appropriate, predetermined volume of the solution or suspension. In the case of a spray this may be achieved for example by means of a metering atomizing spray pump.

The compounds of the present invention may be formulated for aerosol administration, particularly to the respiratory tract and including intranasal administration. The compound will generally have a small particle size for example of the order of 5 microns or less. Such a particle size may be obtained by means known in the art, for example by micronization. The active ingredient is provided in a pressurized pack with a suitable propellant such as a chlorofluorocarbon (CFC) for example dichlorodifluoromethane, trichlorofluoromethane, or dichlorotetrafluoroethane, carbon dioxide or other suitable gas. The aerosol may conveniently also contain a surfactant such as lecithin. The dose of drug may be controlled by a metered valve. Alternatively the active ingredients may be provided in a form of a dry powder, for example a powder mix of the compound in a suitable powder base such as lactose, starch, starch derivatives such as hydroxypropylmethyl cellulose and polyvinylpyrrolidine (PVP). The powder carrier will form a gel in the nasal cavity. The powder composition may be presented in unit dose form for example in capsules or cartridges of e.g., gelatin or blister packs from which the powder may be administered by means of an inhaler.

When desired, formulations can be prepared with enteric coatings adapted for sustained or controlled release administration of the active ingredient.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The pH of the pharmaceutical composition may be any pH suitable for physiological purposes such as between pH 4 and pH 10, preferably between 5 and 8, more preferably around pH 7.

### Method of Producing Antibodies Directed Against the Prodomain of ADAM12

It is another object of the invention to provide a method for producing an antibody directed against the prodomain of ADAM12 as defined above, or a variant or functional equivalent thereof, said method comprising the steps of:
i administering to a mammal a protein comprising the prodomain of ADAM12 or a fragment thereof or a functional equivalent thereof;
ii screening for the ability of said antibody to bind to the prodomain of ADAM12;
iii screening for the ability of said antibody to inhibit the formation of a complex between MMP-14 and/or αVβ3.

Such a method may comprise the step of administering a protein comprising the prodomain of ADAM12 or a fragment thereof or a functional equivalent thereof to a mammal. In some embodiments, the mammal is a rodent. The method may further comprise the steps of isolating cells producing antibody from said mammal, preparing hybridomas from said cells, cultivating the hybridomas and isolating the antibodies produced by the said hybridomas.

Alternatively, the method may rely on antibody production by a cell. Thus in another aspect the invention relates to a method for producing the antibody of the invention, comprising the steps of transfecting a host cell with a nucleic acid construct encoding said antibody. In some embodiments, the antibody is produced by a recombinant cell. In some embodiments, the recombinant cell is a microorganism selected from the group comprising bacteria and eukaryotic microorganisms, such as, but not limited to, yeasts and filamentous fungi. For example, bacteria suitable for the expression of antibodies may be selected from the group comprising Escherichia coli, Lactobacillus zeae, Bacillus subtilis, Streptomyces lividans, Staphylococcus carnosus, Bacillus megaterium and Corynebacterium glutamicum. The microorganism may be a eukaryotic microorganism selected from the group comprising Saccharomyces cerevisiae, Aspergillus niger, Pichia pastoris, Schizosaccharomyces pombe, Yarrowia lipolytica and Kluyveromyces lactis. The recombinant cell may also be a plant cell or an animal cell. The plant cell may be selected from the group comprising Arabidopsis sp., pea, rice, maize, tobacco, barley, or seeds thereof. Suitable animal cells may be animal cell lines derived from a mammal selected from the group comprising Chinese Hamster Ovary, mouse and human. Alternatively, the animal cell may be derived from an insect or from a bird. For example, the cell is a cell line derived from chicken, such as DT40 cells.

The method may further comprise the steps of identifying and selecting the antibody.

### Antibody Recognising an Antibody of the Invention

It is a further object of the invention to provide an antibody which is capable of recognising the antibodies of the invention. Such an antibody may be used for methods such as immunostaining.

### EXAMPLES

### Example 1

### Antibodies and Chemicals

Antibodies against ADAM12 (binding to the prodomain of ADAM12), 6E6 (binding the disintegrin/cycstein.rich domain) and polyclonal rabbit rb122 (raised against the cysteine-rich domain) were previously described (Sundberg et al., 2004, Gilpin et al., 1998). Furthermore, mouse monoclonal antibodies against ADAM12 (7B8, 7C4, 7G3, and 8F10) were generated in this study as described (Sundberg et al., 2004). Briefly, full-length ADAM12-S was produced by HEK293, purified and then used to immunize mice. Hybridomas were generated by fusing mouse spleen cells and a mouse myeloma cell line (NS-1). The single cell hybridomas were expanded and selected for producing ADAM12 mAbs using immunofluorescence. In brief, COS-7 or HEK293 or 293-Vnr cells were transfected with a construct encoding ADAM12-L, conditioned media from the hybridomas was added and visualized by a fluorescence-tagged secondary Ab. Subsequently, the best producing hybridomas were subcloned, and selected for best producing hybridoma using immunofluorescence as described (Sundberg et al., 2004), and Western blotting using cell lysate from cells transfected with constructs encoding ADAM12-L. To determine which domain of ADAM12 each of the antibodies recognized, cos-7 cells were transfected with various truncated versions of ADAM12.

Other antibodies used in the study include mouse monoclonal antibodies against GFP (Clontech Laboratories, Mountain View, Calif., USA), αVβ3 integrin (LM609) (Chemicon/Millipore, Ballerica, Mass., USA), and actin (Calbiochem, Ballerica, Mass., USA), a goat polyclonal antibody against BIK and rabbit polyclonal antibodies against BCL2L11 (Santa Cruz Biotechnology Inc., Santa Cruz, Calif., USA and Nordic Biosite, Täby, Sweden), MMP-14 (Abcam, Cambridge, Mass., USA and LifeSpan Bioscience, Seattle, Wash., USA), and 133 integrin (Santa Cruz Biotechnology Inc). Ki67, Horseradish peroxidase-conjugated secondary goat anti-mouse, goat anti-rabbit, and rabbit anti-goat immunoglobulins were from Dako (Glostrup, Denmark). Alexa Fluor® 488-rabbit anti-goat IgG, Alexa Fluor® 488-goat anti-mouse IgG, Alexa Fluor® 546 F(ab)2 fragment of goat anti-mouse IgG, and Alexa Fluor® 546 F(ab)2 fragment of goat anti-rabbit IgG were from Invitrogen (Naerum, Denmark). GM6001 and TAPI-2 were from Calbiochem.

### Plasmids

Mammalian expression constructs encoding full-length human ADAM12-L, human ADAM12-L fused to GFP, or human ADAM12-L lacking the cytoplasmic tail (ADAM12Δcyt) used for transfections were previously described (Hougaard et al., 2000; Kawaguchi et al., 2003). A point mutation in the catalytic site (E351Q) of ADAM12 was introduced by Mutagenex (Hillsborough, N J, USA) to generate an expression construct encoding ADAM12Δcyt-E351Q. For retroviral transduction, cDNA encoding ADAM12-Δcyt was inserted into pRevTRE (Clontech BD Sciences, Brøndby, Denmark).

### Cell Culture, Transfections, FACS, and Biotinylation of Cell Surface Proteins

The HEK293 cell line stably expressing αVβ3 integrin, called 293-VnR, has been previously described (Sanjay et al., 2001). HEK293, MCF7 and MDA-MB-231 were from ATCC (LGC Standards AB, Boräs, Sweden), cultured as described (Albrechtsen et al., 2011; Frohlich et al., 2011), and transiently transfected using FuGENE® 6 Transfection Reagent (Roche Applied Science, Hvidovre, Denmark). Gelatinase-depleted FBS was used in the some culture medium and performed as described (Kang et al., 2000). ADAM12Δcyt in the pRevTRE vector was stably transduced into MCF7 Tet-Off (Clontech BD Sciences) as described previously (Ronnov-Jessen et al., 2002). The stable MCF7-A12Δcyt cell line was kept in growth media supplied with 50 μg/ml hygromycin B (Roche Applied Science) and 100 μg/ml geneticin (Sigma). To silence ADAM12 expression in the MCF7-A12Δcyt cell line, 100 ng/ml doxycycline (Sigma-Aldrich) was added to the growth media. Small interfering RNAs (siRNAs) against MMP-14 and ADAM12 were obtained as siGENOME® SMARTpool reagents from Thermo Scientific Dharmacon® (Lafayette, Colo., USA), and siRNA universal negative control was from Sigma-Aldrich. siRNA transfection was performed using OPTI-MEMO I and Lipofectamine™ 2000 (Invitrogen). FACS analysis and biotinylation of cell surface proteins was performed as previously described as previously described (Lydolph et al., 2009; Stautz et al., 2012).

### Immunofluorescence Staining

Visualization of ADAM12 was described earlier (Albrechtsen et al., 2011). For visualization of MMP-14 and αVβ3 integrin, the cells were fixed in paraformaldehyde, blocked (1% bovine serum albumin and 1% normal goat serum), and permeabilized or not (0.5% Triton-X 100) before primary and secondary antibodies and 4′,6-diamidino-2-phenylindole (DAPI [Invitrogen], 1:5000) were added. In cytospin experiments, the cell surface staining for ADAM12 and MMP-14 was performed with similar method as described by (Kawaguchi et al., 2003). In brief, the cells were trypsinized and stained without permeabilization, fixed in 4% paraformaldehyde and spun in a cytospin centrifuge (Sandon, Thermo Fisher Scientific Inc, IL 60133, USA). ADAM12 and MMP-14 stained cells were counted using the MetaMorph software with multi wavelength cell scoring program.

For colocalization at the cell surface, Duolink® reagents from Olink (Uppsala, Sweden) were used on non-permeabilized cells. In brief, the Duolink assay is based on the in situ proximity ligation assay (PLA) technique, where two primary antibodies raised in different species are allowed to bind their respective target antigen (i.e. ADAM12, MMP-14, or αVβ3 integrins. Species-specific secondary antibodies, each with a unique short DNA strand attached to it, bind to the primary antibodies and when in close proximity, the DNA strands interact, get amplified and labeled with complementary fluorescent probes visible as distinct dots in the fluorescence microscope. Fluorescence imaging was performed using a confocal laser-scanning microscope (LSM510 Meta, Carl Zeiss, Oberkochen, Germany) equipped with a 63x/1.4 Plan-Apochromat water immersion objective or an inverted Zeiss Axiovert 220 Apotome system with the same type of objectives. The images were processed using the Axiovision program (Carl Zeiss) and MetaMorph software.

### In Situ Gelatinase, 3-D Collagen Assays, and Gelatin Zymography

Cells were seeded on dishes coated with gelatin (10 μg/ml) coupled to Oregon Green® 488 dye (G-13186) from Molecular Probes (Life Technologies, Naerum, Denmark). Twenty hours after cell seeding (unless otherwise stated), gelatin degradation was quantified by measuring the degraded area in μm² (observed as black holes in the gelatin fluorescence) by use of MetaMorph software and correlated to the number of cells as well as the number of cells stained for ADAM12. For each experiment more than 1000 cells were counted and same type of experiment was repeated independently at least three times. PureCol (Advanced BioMatrix, San Diego, Calif., USA) solution was used for making the 3-D collagen gels according to the manufacturer's protocol and cells were embedded and grown as described (Maquoi et al., 2012). Gelatin zymography was performed as previously described (Tatti et al., 2008).

### Detection of Apoptotic and Proliferative Cells

The MetaMorph® Microscopy Automation & Image Analysis Software was used for automatic nuclei counting for detection of apopototic cell bodies and cell proliferation (Universal Imaging Corporation, Downingtown, Pa., USA). The ApopTag® Peroxidase ISOL Apoptosis Detection Kit (Millipore) was used on cultured cells or on paraffin sections from mouse tumour tissue. Apoptosis was also evaluated by counting the percentage of the number of cells with chromatin condensation and nuclear fragmentation stained with DAPI (apoptotic bodies). At least 500 cells were examined in each sample to quantify apoptosis. Parallel paraffin sections of mouse tumours were stained for Ki67 (Dako) to estimate cell proliferation.

### Immunoprecipitation and Western Blot Analysis

Immunoprecipitation and Western blots of cell lysates and tumour tissue were performed as described previously (Frohlich et al., 2011; Stautz et al., 2012).

Quantitative Polymerase Chain Reaction (qPCR)

Total RNA was extracted and isolated from cell lines and qPCR was performed with primers as previously described (Frohlich et al., 2011; Pennington and Edwards, 2010).

### In Vivo Tumourigenic Assay

Equal amounts of MCF7-A12Δcyt cells were injected orthotopically into the mammary gland of 6-8-week-old NOD.Cg-Prkdc^{scid} I12rg^{tm1WjI}/SzJ mice (The Jackson Laboratory, Bar Harbor, Me., USA). Two experiments were performed using 2 different concentrations of tumour cells: 1×10⁶ and 3×10⁶ cells per mouse. One week prior to tumour-cell injection and during the rest of the experiment, mice were given 0.667 μg/ml estradiol-17β (Sigma-Aldrich) in their drinking water. Some mice injected with MCF7-A12Δcyt cells also received 2 mg/ml doxycycline (Sigma-Aldrich) in their drinking water. Tumour size (length and width) was measured over time. Mice were sacrificed as soon as 1 mouse displayed a 1.2 cm² tumour, and tissue dissected as described (Frohlich et al., 2011; Kveriborg et al., 2005). All experiments were conducted in accordance with the guidelines of the Animal Experiment Inspectorate, Denmark.

### Patient Population and Data Analysis

Raw data (CEL files) from the following datasets were downloaded from Array Express (GSE2034, GSE5327, GSE7390, GSE11121) and are available at Gene Expression Omnibus (GEO, http://www.ncbi.nlm.nih.gov/geo/). The 4 datasets from 4 different cohorts (EMC286, Erasmus, TRANSBIG, and Mainz; Desmedt et al., 2007; Minn et al., 2007; Schmidt et al., 2008; Wang et al., 2005) was selected as described previously (Li et al., 2012). All data were normalized together using the RMA (Robust Multichip Average) approach, and calculations were performed using the Partek Genomics Suite 6.6 (Saint Louis, Mo., USA). All calculations and representation of data are on log 2 transformed values. ER status and triple-negative breast cancer were assessed by the gene expression profile. A simple linear-regression analysis was performed in Qlucore Omics Explorer 2.2 (Lund, Sweden), where the expression level of ADAM12 (213790_at) was correlated to the expression of MMP-14 (202828_s_at) and MMP-2 (201069_at). Pearson correlations were performed using GraphPad Prism.

### Statistical Analysis

All assays were performed independently at least 3 times. Statistical analysis was done using Excel or GraphPad Software with the Student's t-test for comparing 2 independent groups or Fisher's exact test. The association between gelatin degradation and antibody inhibition was analyzed using the analysis of variance (ANOVA). P-values of <0.05 were considered statistically significant.

### Example 2

### MMP-14 Recruitment to the Tumour Cell Surface is Stimulated by ADAM12

To investigate if ADAM12 affects recruitment of MMP-14 to the cell surface, we took advantage of a HEK293 cell line that stably expresses αVβ3 integrin, called 293-VnR (Sanjay et al., 2001). Immunostaining of endogenous MMP-14 in 293-VnR cells demonstrated a dot-like localization close to the nucleus in nearly 90% of the cells, while very few cells exhibited MMP-14 staining at the cell surface (FIG. 1A, upper panels and FIG. 1B). However, upon transfection with ADAM12Δcyt, which lacks the cytoplasmic tail and therefore is readily directed to the cell surface (Hougaard et al., 2000), cells exhibited MMP-14 immunostaining at the cell surface (FIG. 1A, lower panels), and the number of cells with dot-like MMP-14 immunostaining decreased significantly (FIG. 1B). The effect of ADAM12 on MMP-14 immunolocalization was subsequently assessed in cytospin experiments, where specifically cell-surface localization is analyzed. Cells transfected with ADAM12Δcyt fluorescent protein (GFP) compared with control vector-GFP exhibited an approximately 10-fold increase in the number of cells with MMP-14 cell surface staining (FIG. 10). Finally, in a biotinylation assay and fluorescence-activated cell sorting (FACS) analysis, we demonstrated that cell-surface MMP-14 could be detected in ADAM12Δcyt-transfected 293-VnR cells, but not in control-transfected cells (FIG. 1D, FIG. 2A). We did not observe changes in expression levels of endogenous MMP-14 in total cell lysate when overexpressing ADAM12Δcyt compared with control cells (FIG. 1D). Taken together, these data support the finding that ADAM12 stimulates recruitment of MMP-14 to the cell surface of 293-VnR cells.

To explore the functional relevance of combined expression of ADAM12 and MMP-14 in a cancer setting, we first sought to determine whether ADAM12 also stimulates MMP-14 translocation to the cell surface in cancer cell lines, using the low-invasive and non-metastatic breast-cancer cell line MCF7. Wild-type MCF7 cells express little or no ADAM12 (FIG. 1E); hence, we induced the expression of ADAM12 (MCF7-A12Δcyt) using a Tet-Off system. MCF7-A12Δcyt cells growing without doxycycline expressed ADAM12 (FIG. 1E), and adding doxycycline to the growth media switched off ADAM12 expression (MCF7-A12Δcyt+dox). In contrast to earlier publications (Deryugina et al., 2000; Figueira et al., 2009; Maquoi et al., 2012), we were able to detect equal levels of MMP-14 under all 3 experimental conditions (FIG. 1E). MMP-14 immunolocalization at the cell surface was analyzed without membrane permeabilization, and the number of cells exhibiting MMP-14 cell-surface staining was increased more than 10-fold in MCF7-A12Δcyt cells as compared with MCF7 and MCF7-A12Δcyt+dox cells (FIG. 1F). These data was confirmed by FACS analysis (FIG. 2B)

Next, we investigated whether endogenously expressed ADAM12 would influence the cellular distribution of MMP-14. For this purpose, we used the invasive breast-cancer cell line MDA-MB-231, which has been previously shown to express ADAM12 (Solomon et al., 2010). We confirmed cell-surface expression of ADAM12 by cytospin experiments, and furthermore showed the presence of MMP-14 in juxtaposition to ADAM12 at the cell surface (FIG. 1G). siRNA knockdown of ADAM12 in MDA-MB-231 cells caused a significant reduction in ADAM12 mRNA levels (FIG. 1H) MMP-14 immunolocalization at the cell surface was analyzed without membrane permeabilization, and the number of cells exhibiting MMP-14 cell-surface staining was significantly lower in MDA-MB-231 cells treated with ADAM12 siRNA than control treated cells (FIG. 1I). These data were confirmed by FACS analysis (FIG. 2C). Notably, the level of MMP-14 mRNA was not affected by knockdown of ADAM12 (FIG. 1H). These data further confirm that ADAM12 may regulate the recruitment of MMP-14 to the tumour-cell surface.

### Example 3

### ADAM12- and MMP-14 Co-Localization at the Tumour Cell Surface Induces Gelatin Degradation

The spatial relationship between ADAM12 and MMP-14 at the cells surface was analyzed using the Duolink technique (briefly described in Material and Methods) in the 3 cell lines examined in FIG. 1. The Duolink experiments were performed on non-permeabilized cells, and colocalization of ADAM12 and MMP-14 was visualized as bright white dots at the cell surface (FIG. 3A). ADAM12 and MMP-14 colocalized at the cell surface independently of whether ADAM12 was exogenously or, as in MDA-MB-231 cells, endogenously expressed (FIG. 3A).

Next, we asked whether the ADAM12-mediated change in subcellular localization of MMP-14 resulted in altered biological activities characteristic of MMP-14. A gelatinolytic assay was used to test the matrix-degradation activity of 293-VnR cells under various experimental conditions. Gelatin degradation was defined as disappearance of gelatin fluorescence, leaving behind black areas underneath the cells. ADAM12Δcyt-transfected cells were able to degrade gelatin, whereas cells not expressing ADAM12 (i.e. non-transfected cell or cells transfected with vector control) showed low or no gelatin degradation (FIG. 3B). The gelatin degradation increased substantially with increasing amount of ADAM12 plasmid used for the transfection (FIG. 4A). In cultures expressing ADAM12, gelatin degradation was detectable after 4 hours in culture, but increased about 5 times after 20 hours (FIG. 3C). The need for ADAM12 and MMP-14 to translocate to the cell surface for the gelatin degradation to occur was further tested by transient transfecting 293-VnR cells with full-length ADAM12-L, which is mostly retained intracellularly (Hougaard et al., 2000), and showed less efficient gelatin degradation than cells with ectopic expression of ADAM12Δcyt, which readily locates to the cell surface (FIG. 4B).

We next analyzed whether the ADAM12-dependent change in the subcellular localization of MMP-14 in breast carcinoma cell lines, as shown in FIG. 1, influenced their ability to degrade gelatin. MCF7-A12Δcyt cells exhibited significant gelatin degradation after 48 hours, whereas the wild-type MCF7 and MCF7-A12Δcyt+dox cells showed little tendency to degrade gelatin in this timeframe (FIG. 3D, E). MDA-MB-231 cells are known to degrade gelatin per se (Yamaguchi et al., 2009). Here we demonstrated that siRNA knockdown of endogenous ADAM12 in MDA-MB-231 cells significantly decreased gelatin degradation (FIG. 3F, G).

Taken together, these data suggest that ADAM12 and MMP-14 colocalize at the tumour cell surface and that ADAM12 is an important regulator of gelatin degradation in breast cancer cell lines.

### Example 4

### ADAM12-Induced Gelatin Degradation is Caused by MMP-14

We next asked which protease is responsible for the ADAM12-induced gelatin degradation. To test whether the catalytic activity of ADAM12 itself was required for the observed gelatin degradation, we transfected 293-VnR cells with ADAM12Δcyt containing a catalytic site mutation (E351Q). Gelatin degradation was similar after transfection with wild-type ADAM12Δcyt and ADAM12ΔcytE351Q, indicating that ADAM12 is not itself responsible for the gelatin degradation (FIG. 5A). In order to confirm that the gelatin degradation results from a metalloprotease activity, we treated cells grown on gelatin with GM6001 or TAPI-2, which are both broad inhibitors of metalloproteases (Black et al., 1997; Gijbels et al., 1994; Moss and Rasmussen, 2007). Both GM6001 and TAPI-2 caused a significant reduction in gelatin degradation (FIG. 5B). Subsequently, to determine whether MMP-14 is involved, we performed siRNA knockdown of MMP-14 in ADAM12Δcyt-transfected 293-VnR cells (FIG. 5C). Gelatin degradation was significantly decreased upon MMP-14 knockdown compared with control siRNA (FIG. 5C), indicating that either MMP-14 itself or a MMP-14-activated metalloprotease (for example, MMP-2) is the major contributor to gelatin degradation under these experimental conditions.

It is well established that upon MMP-14 recruitment to the cell surface, MMP-2 becomes activated and degrades gelatin and collagen (Itoh et al., 2008). This led us to test whether MMP-2 was a candidate for the observed gelatin degradation. First, serum-free cell-culture supernatants from transfected 293-VnR cells tested for gelatin degradation were examined for the presence and activity of MMP-2 by zymography (FIG. 5D, upper panel). Only the presence of the inactive proform of MMP-2 could be detected in media from non-transfected 293-VnR cells and no mature form of MMP-2 was observed when the cells were transfected with ADAM12Δcyt or an empty vector control. In contrast, when cells were transfected directly with MMP-14, the active (mature) form of MMP-2 could be detected as evidenced by its size shift in the zymogram (FIG. 5D, upper panel). As seen from the corresponding gelatin degradation experiments (lower panel), gelatin degradation takes place in the ADAM12Δcyt-transfected culture without the activation of MMP-2, whereas in cells transfected with MMP-14 both MMP-2 activation and gelatin degradation are observed. Zymography performed on serum-free cell-culture supernatants from MCF7-A12Δcyt and MCF7-A12Δcyt+dox also showed no activation of MMP-2 (FIG. 5E). Thus, these data together indicate that MMP-2 is not a candidate protease for the ADAM12-induced gelatin degradation.

### Example 5

### αVβ3 Integrin Provides a Link Between ADAM12 and MMP-14-Mediated Gelatin Degradation

We have previously shown that the secreted form of ADAM12 binds αVβ3 integrin on tumour cells (Thodeti et al., 2005b). Moreover, it is well established that MMP-14 associates with αVβ3 integrin at the cell surface, and that the cell surface MMP-14 activity can be regulated by αVβ3 integrin (Borrirukwanit et al., 2007; Deryugina et al., 2004; Galvez et al., 2002). Based on these data we tested parental HEK293 cells, which unlike 293-VnR cells do not exhibit αVβ3 integrin expression, but express MMP-14 to the same extent as 293-VnR cells (FIG. 7A). We were unable to detect surface localization of MMP-14 in non-permeabilized HEK293 expressing ADAM12Δcyt (FIG. 7B), and found no gelatin degradation (FIG. 6A). To test the role of αVβ3 integrin on gelatin degradation in ADAM12Δcyt-transfected 293-VnR cells, we added the inhibitory LM609 antibody against αVβ3 integrin to the cells. Gelatin degradation was significantly decreased in the presence of LM609 (FIG. 6B). These results suggest a role for αVβ3 integrin in the ADAM12-MMP-14 axis. Therefore, to analyze whether membrane-anchored ADAM12 associates with αVβ3 integrin and MMP-14 in a larger complex, we performed immunoprecipitation studies using monoclonal antibodies (mAbs) against ADAM12. ADAM12 coimmunoprecipitated with both MMP-14 and αVβ3 integrin (FIG. 6C). Interestingly, even though we were unable to detect cell surface MMP-14 in HEK293 expressing ADAM12, we still observed a coimmunprecipitation between ADAM12 and MMP-14 proteins (FIG. 7C). This demonstrates that interaction between ADAM12 and MMP-14 occurs in the lysate; however, αVβ3 integrin is a key-player for correct transport of the protein triple complex to the cell surface. In addition, to visualize the colocalization between ADAM12, MMP-14, and αVβ3 integrin at the cell surface, we employed the Duolink technique on non-permeabilized cells 293-VnR cells transfected with ADAM12Δcyt. As shown in FIG. 6D, colocalization at the cell surface was observed between ADAM12 and αVβ3 integrin and between αVβ3 integrin and MMP-14 (FIG. 6D). Because we used ADAM12Δcyt to transfect the cells examined, these data reveal that the interaction between ADAM12, MMP-14, and αVβ3 integrin relates to the extracellular portion of the 3 molecules.

Several reports have demonstrated low or no levels of αVβ3 integrin at the cell surface of MCF7 cells (Deryugina et al., 2000; Figueira et al., 2009; Taherian et al., 2011); however, both FACS analysis and immunostaining showed that overexpression of ADAM12 in MCF7 cells increases cell-surface levels of αVβ3 integrin (FIG. 6E,F). In addition, we confirm previous reports (Borrirukwanit et al., 2007; Taherian et al., 2011) showing that MDA-MB-231 cells express moderate levels of αVβ3 integrin (FIG. 6E). Therefore, to analyze whether ADAM12-dependent activation of MMP-14 was related to αVβ3 integrin function, we treated MCF7-A12Δcyt cell cultures with the αVβ3 integrin inhibitory LM609 antibody. We observed an 80% reduction of gelatin degradation in LM609-treated MCF7-A12Δcyt cell cultures compared with IgG-treated cultures (FIG. 6G). Taken together, these data demonstrate that αVβ3 integrin participates in the ADAM12-induced activation of MMP-14.

### Example 6

### Monoclonal Antibodies Against the ADAM12 Prodomain Inhibit Gelatin Degradation

Monoclonal antibodies (mAbs) directed to human ADAM12 were developed and tested for function-blocking activity in the gelatin-degradation assay. The mAb 6E6, which has previously been shown to cluster ADAM12 at the cell surface (Albrechtsen et al., 2011), had no effect on gelatin degradation in 293-VnR cells transfected with ADAM12Δcyt. Three of the developed mAbs against ADAM12 (7B8 8F8, and 7C4) clearly inhibited gelatin degradation (FIG. 8A, B and FIG. 9A, B). MAbs 8F8 and 7B8 mAbs recognize an epitope in the prodomain of ADAM12 (FIG. 9). In vitro quenched-fluorescent peptide cleavage assay, and cell-based ectodomain shedding assay demonstrates that 8F8 and 7B8 does not elicit an inhibitory effect on the catalytic activity of ADAM12 (data not shown). Nor did these antibodies exert any inhibitory effect on gelatin degradation in 293-VnR cells transfected with MMP-14, instead of ADAM12 (FIG. 9B).

In addition, we tested the effect of mAbs against human ADAM12 on the ability of MCF7-A12Δcyt and MDA-MB-231 cells to degrade gelatin. Using the MCF7-A12Δcyt cells, the mAbs 7B8 and 8F8 exhibited a significant inhibitory effect on the gelatin degradation, as compared to mouse control IgG (FIG. 8C, D). Similarly, in MDA-MB-231 cells, gelatin degradation was significantly inhibited when using 7B8 and 8F8 mAbs (FIG. 8E, F).

### Example 7

### ADAM12 Protects Breast Tumour Cell Lines from Collagen-Induced Apoptosis

A recent study demonstrated that MMP-14 protects breast-cancer cells from type I collagen-induced apoptosis (Maquoi et al., 2012). We have previously shown that overexpression of ADAM12, both in vivo and in vitro, confers decreased tumour-cell apoptosis (Kveiborg et al., 2005). In light of these results, we wanted to test whether ADAM12-mediated activation of MMP-14 could protect MCF7 cells against type I collagen-induced apoptosis. To this end, MCF7, MCF7-A12Δcyt, and MCF7-A12Δcyt+dox cells were submerged in type I collagen for 6-7 days and examined for morphological characteristics of apoptotic cells (e.g., membrane blebbing). MCF7 cells expressing ADAM12 (MCF7-A12Δcyt) remained round with a distinct cell border, whereas MCF7 cells not expressing ADAM12 (MCF7 and MCF7-A12Δcyt+dox) displayed membrane blebbing, indicating occurrence of apoptosis (data not shown). To analyze the frequency of apoptotic cells in the 3 MCF7 cell lines, apoptotic bodies were counted in cells recovered from the 3-D collagen gels. A representative image of apoptotic bodies in MCF7 cells is shown in FIG. 12A. The percent of apoptotic bodies was significantly reduced in MCF7-A12Δcyt compared with MCF7 and MCF7-A12Δcyt+dox cells (FIG. 11A). The observation, that overexpression of ADAM12 protects MCF7 cells from apoptosis, was confirmed using ApopTag staining (FIG. 12B). MMP-14 has been previously shown to prevent upregulation of the proapoptotic Bcl-2-interacting killer protein (BIK) (Maquoi et al., 2012). Initially, we analysed the protein level of BIK and another pro-apoptotic Bcl-2-interacting protein (BCL2L11; BIM) in the three MCF7 cell lines grown in 2-D cultures. While no changes in BCL2L11 levels was observed between the three MCF cell lines, we found a slight down regulation of BIK in MCF7-A12Δcyt (FIG. 12C). However, consistent with (Maquoi et al., 2012), we observed a significant downregulation of BIK, as well as BCL2L11 in MCF7-A12Δcyt cells grown in 3-D collagen gels compared to MCF7 and MCF7-A12Δcyt+dox controls cells. This suggests that ADAM12 in 2-D cultures primes the cells to obtain an anti-apoptotic phenotype, which seems even more pronounced when the cells are challenged, such as when grown in 3-D collagen gels (FIG. 11B). Taken together, this suggests that ADAM12, through regulation of MMP-14, BIK, and BCL2L11 activity, protects MCF7 cells from programmed cell death (FIG. 11B). The apoptosis-protective effect induced by ADAM12 in MCF7-A12Δcyt cells could be decreased by incubating those cells with mAb 8F8 against ADAM12 (FIG. 11C and FIG. 12B). Similarly, using the metalloprotease inhibitor GM6001, which has no effect on ADAM12 but inhibits MMP-14 activity, significantly increased the fraction of apoptotic bodies in MCF7-A12Δcyt cells, whereas no effects of mAb 8F8 or GM6001 were observed in wild-type MCF7 and MCF7-A12Δcyt+dox cells (FIG. 11C and FIG. 12B).

Maquoi et al. have previously shown that MDA-MB-231 cells exhibit very low levels of apoptotic cells in 3-D collagen cultures (Maquoi et al., 2012). Thus, we asked whether inhibition of ADAM12 activity would influence apoptosis of MDA-MB-231 cells grown in 3-D collagen gel. Indeed, MDA-MB-231 cells incubated with mAb against ADAM12 (8F8) had significantly increased levels of apoptotic bodies compared with control cells (FIG. 11D). Also ApopTag staining showed a decrease in the number of apoptotic cells in MCF7-A12Δcyt compared to control cells (FIG. 12C). GM6001 similarly significantly increased the fraction of apoptotic bodies in MDA-MB-231 cells (FIG. 11D, FIG. 12D). To further dissect a role for MMP-14 in ADAM12-mediated protection against apoptosis, we transiently transfected MDA-MB-231 cells with siRNA against MMP-14. 3-D collagen cultures of MDA-MB-231 cells, which showed efficient knockdown of MMP-14 (FIG. 11E), displayed a significant increase in the percentage of apoptotic bodies (FIG. 11F). Collectively, these data suggest that ADAM12-induced MMP-14 activity protects cancer cells from undergoing programmed cell death. In line with these findings, antibody 8F8 showed no effect on cellular growth in vitro. Thus, antibodies directed against the prodomain of ADAM12 would inhibit interaction between ADAM12 and MMP-14 and thus relieve inhibition.

### Example 8

### ADAM12 Decreases the Apoptotic Capacity of Breast Tumour Cells in Vivo

Based on previous studies demonstrating that both ADAM12 and MMP-14 influence tumour cell apoptosis in vivo (Kveiborg et al., 2005; Maquoi et al., 2012; Roy et al., 2011), we hypothesized that ADAM12 could regulate the apoptotic capacity in a mouse model of breast cancer through regulation of MMP-14 activity. Hence, MCF7-A12Δcyt cells were orthotopically injected into the mammary glands of 6-8-week-old NOD.Cg-Prkdc mice. Overexpression of ADAM12 in MCF7 cells resulted in a significantly higher tumour burden compared with control MCF7-A12Δcyt+dox mice (which were also injected with MCF7-A12Δcyt cells, but then administered doxycycline in their drinking water) (FIG. 13A). Western blotting confirmed the expression of ADAM12 in MCF7-A12Δcyt tumours but not in MCF7-A12Δcyt+dox tumours (FIG. 13B). Verifying previous studies with ADAM12-S and ADAM12-L (Roy et al., 2011), increased expression of ADAM12Δcyt in MCF7 tumours did not affect tumour-cell proliferation, as determined by Ki67 staining (FIG. 13C). In contrast, ApopTag staining revealed a significant decrease in the number of apoptotic tumour cells in MCF7-A12Δcyt-inoculated mice compared with MCF7-A12Δcyt+dox mice (FIG. 13D). To evaluate the influence of ADAM12 on MMP-14 activation, we analyzed the level of the partial autodigested 43 kDa fragment of MMP-14, which is indicative of active MMP-14 (Cho et al., 2008; Ellerbroek et al., 2001; Itoh, 2006), by Western blot (FIG. 13E). As determined by quantification of the band intensities, we observed a significantly increased level of the 43 kDa fragment of MMP-14 in MCF7-A12Δcyt tumours compared with control MCF7-A12Δcyt+dox tumours (FIG. 13F). These in vivo data suggest that the effect of ADAM12 on tumour progression is mediated through decreased apoptosis, possibly through an increased activity of MMP-14.

### Example 9

### Expression of ADAM12 Correlates with MMP-14 and MMP-2 Expression in Human Breast Cancer

Our present results, obtained from cell cultures and mouse studies, suggest that increased levels of both ADAM12 and MMP-14 in breast tumours would be an advantage for tumour progression. Therefore, we aimed to investigate the correlation between ADAM12 and MMP-14 expression in human breast tumours. We combined gene expression profile datasets from 4 different cohorts, as described in Materials and Methods, for a total of 733 human breast-tumour samples. When all tumours, which were taken from lymph node-negative patients who had not received adjuvant chemotherapy, were analyzed together, we found a positive correlation between ADAM12 and MMP-14 expression (FIG. 14A). This positive correlation was retained in estrogen receptor (ER)-positive tumours and in triple-negative breast tumours (TNBC), a subtype of breast cancer that lacks expression of ER, progesterone receptor, and epidermal growth factor receptor (ERBB2) (FIG. 14A). Interestingly, we found an even stronger correlation between ADAM12 and MMP-2. This positive correlation was found in all tumours, in ER-positive tumours, and in TNBC (FIG. 14B). These data suggest that ADAM12, as a modulator of MMP-14 activity, might be biologically relevant in human breast tumours.

### Example 10

### Anti-ADAM12 Antibodies in a Clinical Setting

A 41-year-old woman identifies a mass in her left breast. During self-examination, the patient initially notices a tiny nodule, which doubled in size during the last two months before presentation. Mammography confirms the presence of a mass, 2.5 cm in diameter. CT scans of the chest and abdomen reveals no masses in the lungs, liver, adrenal glands, kidneys, spleen, or ovaries. A bone scan is negative as well.

The patient undergoes a modified radical mastectomy to remove the tumor, including axillary lymph node dissection. 14 lymph nodes are removed in which one is completely replaced by tumor cells, and three others show microscopic involvement. Immunostaining of the tumor reveals a triple-negative tumor by which the carcinoma cells stains negative for HER2/neu (ERBB2), estrogen, and progesterone receptor. However, using immunostaining, the tumor cells stain positive for both MMP-14 and ADAM12 expression.

Based on clinical staging, the patient is given adjuvant radiation therapy to the left breast and axilla. In addition, the patient is given adjuvant therapy which includes a combination of chemotherapy and targeted therapy using monoclonal antibodies against ADAM12.

The adjuvant therapy is being administered according to one of the following doses and schedules for a total of 52 weeks of ADAM12 mAbs therapy:

During and following paclitaxel, docetaxel, or docetaxel/carboplatin: Initial dose of 5 mg/kg as an intravenous infusion over 90 minutes, then at 3 mg/kg as an intravenous infusion over 30 minutes weekly during chemotherapy for the first 12 weeks (paclitaxel or docetaxel) or 18 weeks (docetaxel/carboplatin).

One week following the last weekly dose of ADAM12 mAbs, administration of ADAM12 mAbs at 6 mg/kg as an intravenous infusion over 30-60 minutes every three weeks.

### Example 11

### Sequences

| SEQ ID NO. 1: | |
|---|---|
| amino acid sequence of Homo sapiens ADAM12-L | |
| 10 20 30 40 50 60 | |
| MAARPLPVSP ARALLLALAG ALLAPCEARG VSLWNQGRAD EVVSASVGSG DLWIPVKSFD | |
| 70 80 90 100 110 120 | |
| SKNHPEVLNI RLQRESKELI INLERNEGLI ASSFTETHYL QDGTDVSLAR [symbol] VILGHCY | |
| 130 140 150 160 170 180 | |
| YHGHVRGYSD SAVSLSTCSG LRGLIVFE [symbol] [symbol] YVLEPMKSA TNRYKLFPAK KLKSVRGSCG | |
| 190 200 210 220 230 240 | |
| SHHNTPNLAA KNVFPPPSQT WARRHKRETL KATKYVELVI VADNREFQRQ GKDLEKVKQR | |
| 250 260 270 280 290 300 | |
| LIEIANHVDK FYRPLNIRIV LVGVEVWNDM DKCSVSQDPF TSLHEFLDWR KMKLLPRKSH | |
| 310 320 330 340 350 360 | |
| DNAQLVSGVY FQGTTIGMAP IMSMCTADQS GGIVMDHSDN PLGAAVTLAH ELGHNFGMNH | |
| 370 380 390 400 410 420 | |
| DTLDRGCSCQ MAVEKGGCIM NASTGYPFPM VFSSCSRKDL ETSLEKGMGV CLFNLPEVRE | |
| 430 440 450 460 470 480 | |
| SFGGQKCGNR FVEEGEECDC GEPEECMNRC CNATTCTLKP DAVCAHGLCC EDCQLKPAGT | |
| 490 500 510 520 530 540 | |
| ACRDSSNSCD LPEFCTGASP HCPANVYLHD GHSCQDVDGY CYNGICQTHE QQCVTLWGPG | |
| 550 560 570 580 590 600 | |
| AKPAPGICFE RVNSAGDPYG NCGKVSKSSF AKCEMRDAKC GKIQCQGGAS RPVIGTNAVS | |
| 610 620 630 640 650 660 | |
| IETNIPLQQG GRILCRGTHV YLGDDMPDPG LVLAGTKCAD GKICLNRQCQ NISVFGVHEC | |
| 670 680 690 700 710 720 | |
| AMQCHGRGVC NNRKNCHCEA HWAPPFCDKF GFGGSTDSGP IRQADNQGLT IGILVTILCL | |
| 730 740 750 760 770 780 | |
| LAAGFVVYLK RKTLIRLLFT NKKTTIEKLR CVRPSRPPRG FQPCQAHLGH LGKGLMRKPP | |
| 790 800 810 820 830 840 | |
| DSYPPKDNPR RLLQCQNVDI SRPLNGLNVP QPQSTQRVLP PLHRAPRAPS VPARPLPAKP | |
| 850 860 870 880 890 900 | |
| ALRQAQGTCK PNPPQKPLPA DPLARTTRLT HALARTPGQW ETGLRLAPLR PAPQYPHQVP | |
| RSTHTAYIK | |
| Signal peptide: | 1-28 |
| Prodomain: | 29-206 |
| Metalloprotease: | 207-417 |
| Disintegrin: | 418-512 |
| Cysteine-rich: | 513-588 |
| Fusion-like: | 589-614 |
| EGF-like: | 615-708 |
| Transmembrane: | 709-729 |
| Cytoplasmic tail: | 730-909 |
| N-glycosylation sites in the prodomain: | 111-113 (NYT) |
| | 149-151 (NES) |

| SEQ ID NO. 2: | |
|---|---|
| Amino acid sequence of the prodomain of ADAM12-L, corresponding | |
| to amino acids 29 to 206 of SEQ ID NO: 1. | |
| 10 20 30 40 50 60 | |
| RGVSLWNQGR ADEVVSASVG SGDLWIPVKS FDSKNHPEVL NIRLQRESKE LIINLERNEG | |
| 70 80 90 100 110 120 | |
| LIASSFTETH YLQDGTDVSL AR [symbol] VILGH CYYHGHVRGY SDSAVSLSTC SGLRGLIVFE | |
| 130 140 150 160 170 | |
| [symbol] YVLEPMK SATNRYKLFP AKKLKSVRGS CGSHHNTPNL AAKNVFPPPS QTWARRHK | |
| SEQ ID NO. 3: | |
| Homo sapiens ADAM12-L mRNA | |
| GenBank: AF023476.2 | |
| Kozak consensus | |
| Prodomain sequence | |
| 1 | cactaacgct cttcctagtc cccgggccaa ctcggacagt ttgctcattt attgcaacgg |
| 61 | tcaaggctgg cttgtgccag aacggcgcgc gcgcgacgca cgcacacaca cggggggaaa |
| 121 | cttttttaaa aatgaaaggc tagaagagct cagcggcggc gcgggccgtg cgcgagggct |
| 181 | ccggagctga ctcgccgagg caggaaatcc ctccggtcgc gacgcccggc cccgctcggc |
| 241 | gcccgcgtgg gatggtgcag cgctcgccgc cgggcccgag agctgctgca ctgaaggccg |
| 301 | [symbol] cagcgcgccc gctgcccgtg tcccccgccc gcgccctcct gctcgccctg |
| 361 | gccggtgctc tgctcgcgcc ctgcgaggcc cgaggggtga gcttatggaa ccaaggaaga |
| 421 | gctgatgaag ttgtcagtgc ctctgttcgg agtggggacc tctggatccc agtgaagagc |
| 481 | ttcgactcca agaatcatcc agaagtgctg aatattcgac tacaacggga aagcaaagaa |
| 541 | ctgatcataa atctggaaag aaatgaaggt ctcattgcca gcagtttcac ggaaacccac |
| 601 | tatctgcaag acggtactga tgtctccctc gctcgaaatt acacggtaat tctgggtcac |
| 661 | tgttactacc atggacatgt acggggatat tctgattcag cagtcagtct cagcacgtgt |
| 721 | tctggtctca ggggacttat tgtgtttgaa aatgaaagct atgtcttaga accaatgaaa |
| 781 | agtgcaacca acagatacaa actcttccca gcgaagaagc tgaaaagcgt ccggggatca |
| 841 | tgtggatcac atcacaacac accaaacctc gctgcaaaga atgtgtttcc accaccctct |
| 901 | cagacatggg caagaaggca taaaagagag accctcaagg caactaagta tgtggagctg |
| 961 | gtgatcgtgg cagacaaccg agagtttcag aggcaaggaa aagatctgga aaaagttaag |
| 1021 | cagcgattaa tagagattgc taatcacgtt gacaagtttt acagaccact gaacattcgg |
| 1081 | atcgtgttgg taggcgtgga agtgtggaat gacatggaca aatgctctgt aagtcaggac |
| 1141 | ccattcacca gcctccatga atttctggac tggaggaaga tgaagcttct acctcgcaaa |
| 1201 | tcccatgaca atgcgcagct tgtcagtggg gtttatttcc aagggaccac catcggcatg |
| 1261 | gccccaatca tgagcatgtg cacggcagac cagtctgggg gaattgtcat ggaccattca |
| 1321 | gacaatcccc ttggtgcagc cgtgaccctg gcacatgagc tgggccacaa tttcgggatg |
| 1381 | aatcatgaca cactggacag gggctgtagc tgtcaaatgg cggttgagaa aggaggctgc |
| 1441 | atcatgaacg cttccaccgg gtacccattt cccatggtgt tcagcagttg cagcaggaag |
| 1501 | gacttggaga ccagcctgga gaaaggaatg ggggtgtgcc tgtttaacct gccggaagtc |
| 1561 | agggagtctt tcgggggcca gaagtgtggg aacagatttg tggaagaagg agaggagtgt |
| 1621 | gactgtgggg agccagagga atgtatgaat cgctgctgca atgccaccac ctgtaccctg |
| 1681 | aagccggacg ctgtgtgcgc acatgggctg tgctgtgaag actgccagct gaagcctgca |
| 1741 | ggaacagcgt gcagggactc cagcaactcc tgtgacctcc cagagttctg cacaggggcc |
| 1801 | agccctcact gcccagccaa cgtgtacctg cacgatgggc actcatgtca ggatgtggac |
| 1861 | ggctactgct acaatggcat ctgccagact cacgagcagc agtgtgtcac actctgggga |
| 1921 | ccaggtgcta aacctgcccc tgggatctgc tttgagagag tcaattctgc aggtgatcct |
| 1981 | tatggcaact gtggcaaagt ctcgaagagt tcctttgcca aatgcgagat gagagatgct |
| 2041 | aaatgtggaa aaatccagtg tcaaggaggt gccagccggc cagtcattgg taccaatgcc |
| 2101 | gtttccatag aaacaaacat ccccctgcag caaggaggcc ggattctgtg ccgggggacc |
| 2161 | cacgtgtact tgggcgatga catgccggac ccagggcttg tgcttgcagg cacaaagtgt |
| 2221 | gcagatggaa aaatctgcct gaatcgtcaa tgtcaaaata ttagtgtctt tggggttcac |
| 2281 | gagtgtgcaa tgcagtgcca cggcagaggg gtgtgcaaca acaggaagaa ctgccactgc |
| 2341 | gaggcccact gggcacctcc cttctgtgac aagtttggct ttggaggaag cacagacagc |
| 2401 | ggccccatcc ggcaagcaga taaccaaggt ttaaccatag gaattctggt gaccatcctg |
| 2461 | tgtcttcttg ctgccggatt tgtggtttat ctcaaaagga agaccttgat acgactgctg |
| 2521 | tttacaaata agaagaccac cattgaaaaa ctaaggtgtg tgcgcccttc ccggccaccc |
| 2581 | cgtggcttcc aaccctgtca ggctcacctc ggccaccttg gaaaaggcct gatgaggaag |
| 2641 | ccgccagatt cctacccacc gaaggacaat cccaggagat tgctgcagtg tcagaatgtt |
| 2701 | gacatcagca gacccctcaa cggcctgaat gtccctcagc cccagtcaac tcagcgagtg |
| 2761 | cttcctcccc tccaccgggc cccacgtgca cctagcgtcc ctgccagacc cctgccagcc |
| 2821 | aagcctgcac ttaggcaggc ccaggggacc tgtaagccaa acccccctca gaagcctctg |
| 2881 | cctgcagatc ctctggccag aacaactcgg ctcactcatg ccttggccag gaccccagga |
| 2941 | caatgggaga ctgggctccg cctggcaccc ctcagacctg ctccacaata tccacaccaa |
| 3001 | gtgcccagat ccacccacac cgcctatatt aagtgagaag ccgacacctt ttttcaacag |
| 3061 | tgaagacaga agtttgcact atctttcagc tccagttgga gttttttgta ccaactttta |
| 3121 | ggattttttt taatgtttaa aacatcatta ctataagaac tttgagctac tgccgtcagt |
| 3181 | gctgtgctgt gctatggtgc tctgtctact tgcacaggta cttgtaaatt attaatttat |
| 3241 | gcagaatgtt gattacagtg cagtgcgctg tagtaggcat ttttaccatc actgagtttt |
| 3301 | ccatggcagg aaggcttgtt gtgcttttag tattttagtg aacttgaaat atcctgcttg |
| 3361 | atgggattct ggacaggatg tgtttgcttt ctgatcaagg ccttattgga aagcagtccc |
| 3421 | ccaactaccc ccagctgtgc ttatggtacc agatgcagct caagagatcc caagtagaat |
| 3481 | ctcagttgat tttctggatt ccccatctca ggccagagcc aaggggcttc aggtccaggc |
| 3541 | tgtgtttggc tttcagggag gccctgtgcc ccttgacaac tggcaggcag gctcccaggg |
| 3601 | acacctggga gaaatctggc ttctggccag gaagctttgg tgagaacctg ggttgcagac |
| 3661 | aggaatctta aggtgtagcc acaccaggat agagactgga acactagaca agccagaact |
| 3721 | tgaccctgag ctgaccagcc gtgagcatgt ttggaagggg tctgtagtgt cactcaaggc |
| 3781 | ggtgcttgat agaaatgcca agcacttctt tttctcgctg tcctttctag agcactgcca |
| 3841 | ccagtaggtt atttagcttg ggaaaggtgg tgtttctgta agaaacctac tgcccaggca |
| 3901 | ctgcaaaccg ccacctccct atactgcttg gagctgagca aatcaccaca aactgtaata |
| 3961 | caatgatcct gtattcagac agatgaggac tttccatggg accacaacta ttttcagatg |
| 4021 | tgaaccatta accagatcta gtcaatcaag tctgtttact gcaaggttca acttattaac |
| 4081 | aattaggcag actctttatg cttgcaaaaa ctacaaccaa tggaatgtga tgttcatggg |
| 4141 | tatagttcat gtctgctatc attattcgta gatattggac aaagaacctt ctctatgggg |
| 4201 | catcctcttt ttccaacttg gctgcaggaa tctttaaaag atgcttttaa cagagtctga |
| 4261 | acctatttct taaacacttg caacctacct gttgagcatc acagaatgtg ataaggaaat |
| 4321 | caacttgctt atcaacttcc taaatattat gagatgtggc ttgggcagca tccccttgaa |
| 4381 | ctcttcactc ttcaaatgcc tgactaggga gccatgtttc acaaggtctt taaagtgact |
| 4441 | aatggcatga gaaatacaaa aatactcaga taaggtaaaa tgccatgatg cctctgtctt |
| 4501 | ctggactggt tttcacatta gaagacaatt gacaacagtt acataattca ctctgagtgt |
| 4561 | tttatgagaa agccttcttt tggggtcaac agttttccta tgctttgaaa cagaaaaata |
| 4621 | tgtaccaaga atcttggttt gccttccaga aaacaaaact gcatttcact ttcccggtgt |
| 4681 | tccccactgt atctaggcaa catagtattc atgactatgg ataaactaaa cacgtgacac |
| 4741 | aaacacacac aaaagggaac ccagctctaa tacattccaa ctcgtatagc atgcatctgt |
| 4801 | ttattctata gttattaagt tctttaaaat gtaaagccat gctggaaaat aatactgctg |
| 4861 | agatacatac agaattactg taactgatta cacttggtaa ttgtactaaa gccaaacata |
| 4921 | tatatactat taaaaaggtt tacagaattt tatggtgcat tacgtgggca ttgtcttttt |
| 4981 | agatgcccaa atccttagat ctggcatgtt agcccttcct ccaattataa gaggatatga |
| 5041 | accaaaaaaa aaaaaaaaaa aa |
| Homo sapiens MMP-14 | |
| Uniprot: P50281 | |
| SEQ ID NO. 4 | |
| 10 20 30 40 50 60 | |
| MSPAPRPPRC LLLPLLTLGT ALASLGSAQS SSFSPEAWLQ QYGYLPPGDL RTHTQRSPQS | |
| 70 80 90 100 110 120 | |
| LSAAIAAMQK FYGLQVTGKA DADTMKAMRR PRCGVPDKFG AEIKANVRRK RYAIQGLKWQ | |
| 130 140 150 160 170 180 | |
| HNEITFCIQN YTPKVGEYAT YEAIRKAFRV WESATPLRFR EVPYAYIREG HEKQADIMIF | |
| 190 200 210 220 230 240 | |
| FAEGFHGDST PFDGEGGFLA HAYFPGPNIG GDTHFDSAEP WTVRNEDLNG NDIFLVAVHE | |
| 250 260 270 280 290 300 | |
| LGHALGLEHS SDPSAIMAPF YQWMDTENFV LPDDDRRGIQ QLYGGESGFP TKMPPQPRTT | |
| 310 320 330 340 350 360 | |
| SRPSVPDKPK NPTYGPNICD GNFDTVAMLR GEMFVFKERW FWRVRNNQVM DGYPMPIGQF | |
| 370 380 390 400 410 420 | |
| WRGLPASINT AYERKDGKFV FFKGDKHWVF DEASLEPGYP KHIKELGRGL PTDKIDAALF | |
| 430 440 450 460 470 480 | |
| WMPNGKTYFF RGNKYYRFNE ELRAVDSEYP KNIKVWEGIP ESPRGSFMGS DEVFTYFYKG | |
| 490 500 510 520 530 540 | |
| NKYWKFNNQK LKVEPGYPKS ALRDWMGCPS GGRPDEGTEE ETEVIIIEVD EEGGGAVSAA | |
| 550 560 570 580 | |
| AVVLPVLLLL LVLAVGLAVF FFRRHGTPRR LLYCQRSLLD KV | |
| Homo sapiens MMP-14, mRNA | |
| NCBI reference sequence: NM_004995.2 | |
| SEQ ID NO. 5 | |
| 1 | cagaccccag ttcgccgact aagcagaaga aagatcaaaa accggaaaag aggagaagag |
| 61 | caaacaggca ctttgaggaa caatcccctt taactccaag ccgacagcgg tctaggaatt |
| 121 | caagttcagt gcctaccgaa gacaaaggcg ccccgaggga gtggcggtgc gaccccaggg |
| 181 | cgtgggcccg gccgcggagc ccacactgcc cggctgaccc ggtggtctcg gaccatgtct |
| 241 | cccgccccaa gacccccccg ttgtctcctg ctccccctgc tcacgctcgg caccgcgctc |
| 301 | gcctccctcg gctcggccca aagcagcagc ttcagccccg aagcctggct acagcaatat |
| 361 | ggctacctgc ctcccgggga cctacgtacc cacacacagc gctcacccca gtcactctca |
| 421 | gcggccatcg ctgccatgca gaagttttac ggcttgcaag taacaggcaa agctgatgca |
| 481 | gacaccatga aggccatgag gcgcccccga tgtggtgttc cagacaagtt tggggctgag |
| 541 | atcaaggcca atgttcgaag gaagcgctac gccatccagg gtctcaaatg gcaacataat |
| 601 | gaaatcactt tctgcatcca gaattacacc cccaaggtgg gcgagtatgc cacatacgag |
| 661 | gccattcgca aggcgttccg cgtgtgggag agtgccacac cactgcgctt ccgcgaggtg |
| 721 | ccctatgcct acatccgtga gggccatgag aagcaggccg acatcatgat cttctttgcc |
| 781 | gagggcttcc atggcgacag cacgcccttc gatggtgagg gcggcttcct ggcccatgcc |
| 841 | tacttcccag gccccaacat tggaggagac acccactttg actctgccga gccttggact |
| 901 | gtcaggaatg aggatctgaa tggaaatgac atcttcctgg tggctgtgca cgagctgggc |
| 961 | catgccctgg ggctcgagca ttccagtgac ccctcggcca tcatggcacc cttttaccag |
| 1021 | tggatggaca cggagaattt tgtgctgccc gatgatgacc gccggggcat ccagcaactt |
| 1081 | tatgggggtg agtcagggtt ccccaccaag atgccccctc aacccaggac tacctcccgg |
| 1141 | ccttctgttc ctgataaacc caaaaacccc acctatgggc ccaacatctg tgacgggaac |
| 1201 | tttgacaccg tggccatgct ccgaggggag atgtttgtct tcaaggagcg ctggttctgg |
| 1261 | cgggtgagga ataaccaagt gatggatgga tacccaatgc ccattggcca gttctggcgg |
| 1321 | ggcctgcctg cgtccatcaa cactgcctac gagaggaagg atggcaaatt cgtcttcttc |
| 1381 | aaaggagaca agcattgggt gtttgatgag gcgtccctgg aacctggcta ccccaagcac |
| 1441 | attaaggagc tgggccgagg gctgcctacc gacaagattg atgctgctct cttctggatg |
| 1501 | cccaatggaa agacctactt cttccgtgga aacaagtact accgtttcaa cgaagagctc |
| 1561 | agggcagtgg atagcgagta ccccaagaac atcaaagtct gggaagggat ccctgagtct |
| 1621 | cccagagggt cattcatggg cagcgatgaa gtcttcactt acttctacaa ggggaacaaa |
| 1681 | tactggaaat tcaacaacca gaagctgaag gtagaaccgg gctaccccaa gtcagccctg |
| 1741 | agggactgga tgggctgccc atcgggaggc cggccggatg aggggactga ggaggagacg |
| 1801 | gaggtgatca tcattgaggt ggacgaggag ggcggcgggg cggtgagcgc ggctgccgtg |
| 1861 | gtgctgcccg tgctgctgct gctcctggtg ctggcggtgg gccttgcagt cttcttcttc |
| 1921 | agacgccatg ggacccccag gcgactgctc tactgccagc gttccctgct ggacaaggtc |
| 1981 | tgacgcccac cgccggcccg cccactccta ccacaaggac tttgcctctg aaggccagtg |
| 2041 | gcagcaggtg gtggtgggtg ggctgctccc atcgtcccga gccccctccc cgcagcctcc |
| 2101 | ttgcttctct ctgtcccctg gctggcctcc ttcaccctga ccgcctccct ccctcctgcc |
| 2161 | ccggcattgc atcttcccta gataggtccc ctgagggctg agtgggaggg cggccctttc |
| 2221 | cagcctctgc ccctcagggg aaccctgtag ctttgtgtct gtccagcccc atctgaatgt |
| 2281 | gttgggggct ctgcacttga aggcaggacc ctcagacctc gctggtaaag gtcaaatggg |
| 2341 | gtcatctgct ccttttccat cccctgacat accttaacct ctgaactctg acctcaggag |
| 2401 | gctctgggca ctccagccct gaaagcccca ggtgtaccca attggcagcc tctcactact |
| 2461 | ctttctggct aaaaggaatc taatcttgtt gagggtagag accctgagac agtgtgaggg |
| 2521 | ggtggggact gccaagccac cctaagacct tgggaggaaa actcagagag ggtcttcgtt |
| 2581 | gctcagtcag tcaagttcct cggagatctg cctctgcctc acctacccca gggaacttcc |
| 2641 | aaggaaggag cctgagccac tggggactaa gtgggcagaa gaaacccttg gcagccctgt |
| 2701 | gcctctcgaa tgttagcctt ggatggggct ttcacagtta gaagagctga aaccaggggt |
| 2761 | gcagctgtca ggtagggtgg ggccggtggg agaggcccgg gtcagagccc tgggggtgag |
| 2821 | cctgaaggcc acagagaaag aaccttgccc aaactcaggc agctggggct gaggcccaaa |
| 2881 | ggcagaacag ccagaggggg caggagggga ccaaaaagga aaatgaggac gtgcagcagc |
| 2941 | attggaaggc tggggccggg caggccaggc caagccaagc agggggccac agggtgggct |
| 3001 | gtggagctct caggaagggc cctgaggaag gcacacttgc tcctgttggt ccctgtcctt |
| 3061 | gctgcccagg cagcgtggag gggaagggta gggcagccag agaaaggagc agagaaggca |
| 3121 | cacaaacgag gaatgagggg cttcacgaga ggccacaggg cctggctggc cacgctgtcc |
| 3181 | cggcctgctc accatctcag tgaggggcag gagctggggc tcgcttaggc tgggtccacg |
| 3241 | cttccctggt gccagcaccc ctcaagcctg tctcaccagt ggcctgccct ctcgctcccc |
| 3301 | cacccagccc acccattgaa gtctccttgg gccaccaaag gtggtggcca tggtaccggg |
| 3361 | gacttgggag agtgagaccc agtggaggga gcaagaggag agggatgtcg ggggggtggg |
| 3421 | gcacggggta ggggaaatgg ggtgaacggt gctggcagtt cggctagatt tctgtcttgt |
| 3481 | ttgttttttt gttttgttta atgtatattt ttattataat tattatatat gaattccaaa |
| 3541 | aaaaaaaaaa aaaaaaaa |

### REFERENCES

Albrechtsen R, Stautz D, Sanjay A, Kveiborg M, Wewer U M (2011) Exp. Cell Res. 317, 195-209.
Bird et al. (1988) Science 242:423-426.
Black R A, Rauch C T, Kozlosky C J, et al. (1997) Nature 385, 729-733.
Borrirukwanit K, Lafleur M A, Mercuri F A, et al. (2007) Matrix biology 26, 291-305.
Cho J A, Osenkowski P, Zhao H, et al. (2008) J. Biol. Chem. 283, 17391-17405.
Coussens L M, Fingleton B, Matrisian L M. (2002) Science. 295(5564):2387-92.
Deryugina El, Ratnikov B I, Yu Q, Baciu P C, Rozanov D V, Strongin A Y (2004) Traffic 5, 627-641.
Desmedt C, Piette F, Loi S, et al. (2007) Clin. Cancer Res. 13, 3207-3214.
Devy L, Huang L, Naa L, et al. (2009) Cancer Res. 69(4):1517-26.
Eckert-Boulet N, Pedersen M L, Krogh B O, Lisby M. (2012) Yeast. 29(8):323-34.
Ellerbroek S M, Wu Y I, Overall C M, Stack M S (2001). J. Biol. Chem. 276, 24833-24842.
Fang L, Barekati Z, Zhang B, Liu Z, Zhong X. (2011) Swiss Med Wkly. 141:w13231. doi: 10.4414/smw.2011.13231.
Figueira R C, Gomes L R, Neto J S, Silva F C, Silva I D, Sogayar M C (2009) BMC Cancer 9, 20.
Fröhlich C, Nehammer C, Albrechtsen R, et al. (2011) Mol Cancer Res. 9(11):1449-61.
Galvez B G, Matias-Roman S, Yanez-Mo M, et al. (2002) J. Cell Biol. 159, 509-521.
Gilpin B J, Loechel F, Mattei M G, Engvall E, Albrechtsen R, Wewer U M (1998) J. Biol Chem. 273(1):157-66.
Gijbels K, Galardy R E, Steinman L (1994) J. Clin. Invest 94, 2177-2182.
Greenberg A S, Avila D, Hughes M, Hughes A, McKinney E C, Flajnik. (1995) Nature. 374, 168-173.
Hamers-Casterman C, Atarhouch T, Muyldermans S, et al. (1993) Nature. 363(6428):446-8.
Hollinger et al. (1993) Proc. Natl. Acad Sci. USA 90: 6444-6448.
Hougaard S, Loechel F, Xu X et al (2000). Biochem. Biophys. Res. Commun. 275, 261-267.
Huston et al. (1988) Proc. Natl. Acad. Sci. USA 85:5879-5883.
Iba K, Albrechtsen R, Gilpin B J, Loechel F and Wewer U M (1999). Am. J. Pathol. 154, 1489-1501.
Itoh Y, Ito N, Nagase H, Seiki M (2008). J. Biol. Chem. 283, 13053-13062.
Itoh Y (2006). IUBMB Life 58, 589-596.
Kang T, Yi J, Yang W et al. (2000) FASEB J. 14, 2559-2568.
Kawaguchi N, Sundberg C, Kveiborg M et al. (2003) J. Cell Sci. 116, 3893-3904.
Kohler and Milstein (1975) Nature 256:495.
Kveiborg M, Frohlich C, Albrechtsen R, et al. (2005) Cancer Res. 65, 4754-4761.
Kveiborg M, Albrechtsen R, Couchman J R, Wewer U M. (2008) 40(9):1685-702.
Li H, Duhachek-Muggy S, Qi Y et al. (2012) Breast Cancer Res. Treat. 135, 759-769.
Lonberg, N. et al. (1994) Nature 368 (6474):856-859.
Lydolph M C, Morgan-Fisher M, Hoye A M, et al. (2009) Exp. Cell Res. 315, 3312-3324.
Maquoi E, Assent D, Detilleux J et al. (2012) Oncogene 31, 480-493.
Minn A J, Gupta G P, Padua D, et a. (2007) Proc. Natl. Acad. Sci. USA 104, 6740-6745.
Moss M L, Bomar M, Liu Q, et al. (2007) J. Biol. Chem. 282, 35712-35721.
Nour-Eldin H H, Hansen B G et al. (2006) Nucleic Acids Res. 34(18):e122.
Pennington C J, Edwards D R (2010). Methods Mol. Biol. 622, 159-173.
Reiter Y, Brinkmann U, et al. (1994) J. Biol. Chem. 269(15):18327-31.
Ronnov-Jessen L, Villadsen R, et al. (2002) Am. J. Pathol. 161, 471-480.
Roy R, Rodig S, Bielenberg D, Zurakowski D, Moses M A (2011) J. Biol. Chem. 286, 20758-20768.
Sanjay A, Houghton A, Neff L (2001) J. Cell Biol. 152, 181-195.
Schmidt M, Bohm D, von Tome C, et al. (2008) Cancer Res. 68, 5405-5413.
Solomon E, Li H, Muggy S D, Syta E, Zolkiewska A (2010) J. Biol. Chem. 285, 21969-21977.
Stautz D, Leyme A, Grandal M V, et al. (2012) Traffic 13, 1532-1546.
Stöcker W, Bode W. (1995) Curr Opin Struct Biol. 5(3):383-90.
Sundberg C, Thodeti C K, Kveiborg M, et al. (2004). J. Biol. Chem. 279, 51601-51611.
Taherian A, Li X, Liu Y, Haas T A (2011). BMC Cancer 11, 293.
Tatti O, Vehvilainen P, Lehti K, Keski-Oja J. (2008). Exp. Cell Res. 314, 2501-2514.
Thodeti, C K, Frohlich C, Nielsen C K, et al. (2005a). Exp. Cell Res. 309, 438-450.
Thodeti C K, Frohlich C, Nielsen C K, et al. (2005b). FEBS lett. 579, 5589-5595.
Villiers B R, Stein V, Hollfelder F. (2010) Protein Eng Des Sel. 23(1):1-8.
Wang Y, Klijn J G, Zhang Y, et al. (2005) Lancet 365, 671-679.
Ward E S, Güssow D, Griffiths A D, Jones P T, Winter G. (1989) Nature 341:544-546.
Wewer U M, Mörgelin M, Holck P et al. (2006) J Biol Chem. 281(14):9418-22.
Wozniak-Knopp G, Stadlmann J, Rüker F (2012) PLoS ONE 7(1): e30083. doi:10.1371/journal.pone.0030083.
Yamaguchi H, Takeo Y, Yoshida S et al. (2009) Cancer Res. 69, 8594-8602.

1. An antibody capable of specifically binding an epitope within the prodomain of ADAM12 (SEQ ID NO: 2), said antibody selected from the group consisting of:
vii the monoclonal antibody produced by the hybridoma cell line 7B8 deposited under the Budapest Treaty with HPA Cultures collection (ECACC) under accession number 13082101;
viii the monoclonal antibody produced by the hybridoma cell line 8F8 deposited under the Budapest Treaty with HPA Cultures collection (ECACC) under accession number 13082102;
ix an antibody capable of binding the same epitope as 7B8 or 8F8;
x an antibody capable of inhibiting binding of 7B8 or 8F8 to ADAM12;
xi an antibody having the V_{H} and V_{L} of 7B8 or 8F8;
xii an antibody having the CDRs of the V_{H} and V_{L} of 7B8 or 8F8.

2. An antibody according to claim 1 for use as a medicament.

3. An antibody or a functional equivalent thereof, capable of specifically recognising and binding an epitope within the prodomain of ADAM12 (SEQ ID NO: 2), wherein said antibody or functional equivalent thereof specifically recognises at least part of an epitope recognised by one or more reference antibodies selected from the group consisting of:
i the monoclonal antibody produced by the hybridoma cell line 7B8 deposited under the Budapest Treaty with HPA Cultures collection (ECACC) under accession number 13082101;
ii the monoclonal antibody produced by the hybridoma cell line 8F8 deposited under the Budapest Treaty with HPA Cultures collection (ECACC) under accession number 13082102;
for use in a method of treatment of cancer in a subject.

4. The antibody for the use according to claim 3, wherein the antibody is a murine monoclonal antibody.

5. The antibody for the use according to any one of claims 3 to 4, wherein the antibody has been humanised.

6. The antibody for the use according to any one of claims 3 to 5, wherein said functional equivalent comprises or consists of an ADAM12 prodomain-binding fragment of said antibody.

7. The antibody according to claim 6, wherein said fragment is selected from the group consisting of Fab, Fab′, F(ab′)₂ and Fv fragments, such as ScFv fragments.

8. The antibody for the use according to any one of claims 3 to 7, wherein said functional equivalent is a small molecule mimic, mimicking an antibody.

9. The antibody for the use according to claims 3 to 8, wherein said antibody or functional equivalent thereof specifically recognises and binds an epitope within the prodomain of ADAM12 that comprises or consists of amino acid residues 1 to 10 of SEQ ID NO: 2, or amino acid residues 11 to 20 of SEQ ID NO: 2, or amino acid residues 21 to 30 of SEQ ID NO: 2, or amino acid residues 31 to 40 of SEQ ID NO: 2, or amino acid residues 41 to 50 of SEQ ID NO: 2, or amino acid residues 51 to 60 of SEQ ID NO: 2, or amino acid residues 61 to 70 of SEQ ID NO: 2, or amino acid residues 71 to 80 of SEQ ID NO: 2, or amino acid residues 81 to 90 of SEQ ID NO: 2, or amino acid residues 91 to 100 of SEQ ID NO: 2, or amino acid residues 101 to 110 of SEQ ID NO: 2, or amino acid residues 111 to 120 of SEQ ID NO: 2, or amino acid residues 121 to 130 of SEQ ID NO: 2, or amino acid residues 131 to 140 of SEQ ID NO: 2, or amino acid residues 141 to 150 of SEQ ID NO: 2, or amino acid residues 151 to 160 of SEQ ID NO: 2, or amino acid residues 161 to 170 of SEQ ID NO: 2, or amino acid residues 171 to 178 of SEQ ID NO: 2.

10. The antibody according to claim 8, wherein said epitope may comprise zero, one or two glycosylation domains of the prodomain.

11. The antibody for the use according to any one of claims 3 to 10, wherein the antibody is constructed by domain shuffling.

12. The antibody for the use according to any one of claims 3 to 11, wherein the antibody is an antibody variant.

13. The antibody variant according to claim 12, wherein the variant has been modified to increase half-life, stability, solubility, and/or bioavailability.

14. The antibody variant according to claim 13, wherein the variant comprises engineered intradomain sulphide bonds.

15. The antibody variant according to claim 13, wherein one or more Fv fragment comprises a peptide linker between the V_{H} and the V_{L} domains.

16. The antibody for the use according to any one of claims 3 to 15, wherein the subject is a mammal.

17. The antibody according to claim 16, wherein the mammal is a human.

18. The antibody for the use according to any one of claims 3 to 17, wherein the cancer is selected from the group consisting of cancer of the breast, bladder, ovary, colon, uterus, cervix, kidney, prostate, oesophagus, renal cells, pancreas, rectum, stomach, squamous cells, lung, head and neck, skin, testicles, liver, oral cavity, brain, bone, bone marrow and blood cells.

19. The antibody for the use according to any one of claims 3 to 18, wherein the cancer is selected from the group consisting of cancer of the breast, bladder, colon, liver, lung, oral cavity, stomach, brain and bone.

20. The antibody for the use according to any one of claims 3 to 19, wherein the cancer is bladder cancer.

21. The antibody for the use according to any one of claims 3 to 17, wherein the cancer is breast cancer.

22. The antibody for the use according to any one of claims 3 to 21, wherein the cancer is characterised by elevated levels of ADAM12.

23. The antibody according to claim 22, wherein the levels of ADAM12 are determined in vivo or in vitro.

24. The antibody according to claim 23, wherein the levels of ADAM12 are determined by measuring mRNA levels or protein levels.

25. The antibody according to claim 24, wherein the mRNA levels are determined by Northern blot, RT-PCR or microarray analysis.

26. The antibody according to claim 24, wherein the protein levels are determined by Western blot or by immunostaining.

27. The antibody for the use according to any one of claims 3 to 26, wherein said antibody is capable of inhibiting gelatin degradation.

28. The antibody for the use according to any one of the claims 3 to 27, wherein said antibody does not inhibit the catalytic activity of ADAM12.

29. The antibody for the use according to any one of the claims 3 to 28, wherein said antibody is capable of inhibiting the MMP-14-induced increase of Bcl2-interacting killer protein.

30. The antibody for the use according to any one of the claims 3 to 29, wherein said antibody induces apoptosis.

31. The antibody for the use according to any one of the claims 3 to 30, wherein said antibody does not affect cell growth.

32. The antibody for the use according to any one of the claims 3 to 31, wherein said antibody is stable in the serum of said subject.

33. The antibody for the use according to any one of the claims 3 to 32, wherein said antibody is not toxic to said subject after administration.

34. A method of treatment of cancer in an individual in need thereof, the method comprising the steps of:
a) providing a sample from tumour tissue of an individual,
b) determining the expression level of ADAM12 in the sample of step a),
c) correlating the expression level of step b) with the expression level of a control tissue,
d) assessing a treatment regime,
e) administering to the individual a therapeutically effective amount of an antibody according to any one of claims 1 to 30.

35. A method of treatment of cancer in an individual in need thereof, the method comprising the steps of:
a) providing a sample from tumour tissue of an individual,
b) determining the degradation level of gelatin in the sample of step a,
c) correlating the expression level of step b with the expression level of a control tissue,
d) assessing a treatment regime,
e) administering to the individual a therapeutically effective amount of an antibody according to any one of claims 1 to 30.

36. A method of treatment of cancer in an individual in need thereof, said method comprising administering an antibody which inhibits gelatin degradation.

37. A method of treatment of cancer in an individual in need thereof, said method comprising administering an antibody directed against the prodomain of ADAM12.

38. Use of the antibody according to any one of claims 1 to 30 for the preparation of a medicament for the treatment of cancer.

39. An antibody capable of selectively recognising and binding the antibody according to any one of claims 1 to 30.

40. A method of inhibiting formation of a complex between ADAM12, MMP-14 and/or αVβ3, said method comprising administering the antibody according to any one of claims 1 to 30.

41. A method for producing the antibody according to claims 1 to 30, comprising the steps of
i administering to a mammal a protein comprising the prodomain of ADAM12 or a fragment thereof or a functional equivalent thereof;
ii screening for the ability of said antibody to bind to the prodomain of ADAM12;
iii screening for the ability of said antibody to inhibit the formation of a complex between MMP-14 and/or αVβ3.

42. The method according to claim 41, wherein the mammal is a rodent.

43. The method according to any one of claim 41 or 42, wherein the method further comprises isolating antibody producing cells from said mammal, preparing hybridoma cells from said antibody producing cells, cultivating said hybridomas and isolating antibodies produced by said hybridomas.

44. A method for producing the antibody according to claims 1 to 30, comprising the steps of transfecting a host cell with a nucleic acid construct encoding said antibody.

45. The method according to claim 44, wherein the antibody is produced by a recombinant cell.

46. The method according to claim 45, wherein the recombinant cell is a microorganism selected from the group comprising bacteria and eukaryotic microorganisms.

47. The method according to claim 46, wherein the microorganism is a bacterium selected from the group comprising Escherichia coli, Lactobacillus zeae, Bacillus subtilis, Streptomyces lividans, Staphylococcus carnosus, Bacillus megaterium and Corynebacterium glutamicum.

48. The method according to claim 46, wherein the microorganism is a eukaryotic microorganism selected from the group comprising Saccharomyces cerevisiae, Aspergillus niger, Pichia pastoris, Schizosaccharomyces pombe, Yarrowia lipolytica and Kluyveromyces lactis.

49. The method according to claim 44, wherein the recombinant cell is selected from the group comprising plant cells and animal cells.

50. The method according to claim 49, wherein the plant cell is selected from the group comprising Arabidopsis sp., pea, rice, maize, tobacco, barley, or seeds thereof.

51. The method according to claim 49, wherein the animal cell is derived from a mammal selected from the group comprising Chinese Hamster Ovary, mouse and human.

52. The method according to claim 49, wherein the animal cell is derived from an insect.

53. The method according to claim 49, wherein the animal cell is derived from an avian cell line.

54. The method according to any one of claims 41 to 53, further comprising the steps of identifying and selecting the antibody.

55. A pharmaceutical composition comprising the antibody according to any one of claims 1 to 30.

56. The pharmaceutical composition according to claim 55 further comprising a pharmaceutically acceptable carrier.

57. The pharmaceutical composition according to any one of claims 55 and 56 wherein the pH of the composition is between pH 4 and pH 10.

58. The pharmaceutical composition according to any one of claims 55 to 57, wherein the composition is formulated for oral ingestion.

59. The pharmaceutical composition according to any one of claims 55 to 57, wherein the composition is formulated for parenteral administration.

60. The pharmaceutical composition according to claim 59, wherein the parenteral administration is by injection.

61. The pharmaceutical composition according to any one of claims 59 to 60, wherein the parenteral administration is intravenous, intramuscular, intraspinal, intraperitoneal, subcutaneous, a bolus or a continuous administration.

62. The pharmaceutical composition according to any one of claims 55 to 61, wherein the administration occurs at intervals of 30 minutes to 24 hours, such as at intervals of 1 to 6 hours, such as three times a day.

63. The pharmaceutical composition according to any one of claims 55 to 62, wherein the duration of the treatment is from 6 to 72 hours.

64. The pharmaceutical composition according to any one of claims 55 to 63, wherein the duration of the treatment is from 24 hours to 7 days.

65. The pharmaceutical composition according to any one of claims 55 to 64, wherein the duration of the treatment is from 4 days to 150 days.

66. The pharmaceutical composition according to any one of claims 55 to 65, wherein the duration of the treatment is lifelong.

67. The pharmaceutical composition according to any one of claims 55 to 66, wherein the dosage of the active ingredient is between 10 μg to 500 mg per kg body mass, such as from 50 μg to 250 mg per kg body mass.

68. A kit comprising the pharmaceutical composition according to any one of claims 55 to 67, and instructions for use.


SEQUENCE LISTING (5 sequences)

SEQ ID NO: 1 - protein, 909 aa, Homo sapiens
  SIGNAL (1)..(28)
  DOMAIN (1)..(909): ADAM12-L
MAARPLPVSPARALLLALAGALLAPCEARGVSLWNQGRADEVVSASVGSGDLWIPVKSFDSKNHPEVLNIRLQRESKELIINLERNEGLIASSFTETHYLQDGTDVSLARNYTVILGHCYYHGHVRGYSDSAVSLSTCSGLRGLIVFENESYVLEPMKSATNRYKLFPAKKLKSVRGSCGSHHNTPNLAAKNVFPPPSQTWARRHKRETLKATKYVELVIVADNREFQRQGKDLEKVKQRLIEIANHVDKFYRPLNIRIVLVGVEVWNDMDKCSVSQDPFTSLHEFLDWRKMKLLPRKSHDNAQLVSGVYFQGTTIGMAPIMSMCTADQSGGIVMDHSDNPLGAAVTLAHELGHNFGMNHDTLDRGCSCQMAVEKGGCIMNASTGYPFPMVFSSCSRKDLETSLEKGMGVCLFNLPEVRESFGGQKCGNRFVEEGEECDCGEPEECMNRCCNATTCTLKPDAVCAHGLCCEDCQLKPAGTACRDSSNSCDLPEFCTGASPHCPANVYLHDGHSCQDVDGYCYNGICQTHEQQCVTLWGPGAKPAPGICFERVNSAGDPYGNCGKVSKSSFAKCEMRDAKCGKIQCQGGASRPVIGTNAVSIETNIPLQQGGRILCRGTHVYLGDDMPDPGLVLAGTKCADGKICLNRQCQNISVFGVHECAMQCHGRGVCNNRKNCHCEAHWAPPFCDKFGFGGSTDSGPIRQADNQGLTIGILVTILCLLAAGFVVYLKRKTLIRLLFTNKKTTIEKLRCVRPSRPPRGFQPCQAHLGHLGKGLMRKPPDSYPPKDNPRRLLQCQNVDISRPLNGLNVPQPQSTQRVLPPLHRAPRAPSVPARPLPAKPALRQAQGTCKPNPPQKPLPADPLARTTRLTHALARTPGQWETGLRLAPLRPAPQYPHQVPRSTHTAYIK

SEQ ID NO: 2 - protein, 178 aa, Homo sapiens
  DOMAIN (1)..(178): Prodomain sequence of ADAM12-L, corresponding to amino acids 29 to 206 of SEQ ID NO 1
RGVSLWNQGRADEVVSASVGSGDLWIPVKSFDSKNHPEVLNIRLQRESKELIINLERNEGLIASSFTETHYLQDGTDVSLARNYTVILGHCYYHGHVRGYSDSAVSLSTCSGLRGLIVFENESYVLEPMKSATNRYKLFPAKKLKSVRGSCGSHHNTPNLAAKNVFPPPSQTWARRHK

SEQ ID NO: 3 - DNA, 5062 bp, Homo sapiens
  mRNA (1)..(5062): ADAM12-L mRNA GenBank AF023476.2
cactaacgctcttcctagtccccgggccaactcggacagtttgctcatttattgcaacggtcaaggctggcttgtgccagaacggcgcgcgcgcgacgcacgcacacacacggggggaaacttttttaaaaatgaaaggctagaagagctcagcggcggcgcgggccgtgcgcgagggctccggagctgactcgccgaggcaggaaatccctccggtcgcgacgcccggccccgctcggcgcccgcgtgggatggtgcagcgctcgccgccgggcccgagagctgctgcactgaaggccggcgacgatggcagcgcgcccgctgcccgtgtcccccgcccgcgccctcctgctcgccctggccggtgctctgctcgcgccctgcgaggcccgaggggtgagcttatggaaccaaggaagagctgatgaagttgtcagtgcctctgttcggagtggggacctctggatcccagtgaagagcttcgactccaagaatcatccagaagtgctgaatattcgactacaacgggaaagcaaagaactgatcataaatctggaaagaaatgaaggtctcattgccagcagtttcacggaaacccactatctgcaagacggtactgatgtctccctcgctcgaaattacacggtaattctgggtcactgttactaccatggacatgtacggggatattctgattcagcagtcagtctcagcacgtgttctggtctcaggggacttattgtgtttgaaaatgaaagctatgtcttagaaccaatgaaaagtgcaaccaacagatacaaactcttcccagcgaagaagctgaaaagcgtccggggatcatgtggatcacatcacaacacaccaaacctcgctgcaaagaatgtgtttccaccaccctctcagacatgggcaagaaggcataaaagagagaccctcaaggcaactaagtatgtggagctggtgatcgtggcagacaaccgagagtttcagaggcaaggaaaagatctggaaaaagttaagcagcgattaatagagattgctaatcacgttgacaagttttacagaccactgaacattcggatcgtgttggtaggcgtggaagtgtggaatgacatggacaaatgctctgtaagtcaggacccattcaccagcctccatgaatttctggactggaggaagatgaagcttctacctcgcaaatcccatgacaatgcgcagcttgtcagtggggtttatttccaagggaccaccatcggcatggccccaatcatgagcatgtgcacggcagaccagtctgggggaattgtcatggaccattcagacaatccccttggtgcagccgtgaccctggcacatgagctgggccacaatttcgggatgaatcatgacacactggacaggggctgtagctgtcaaatggcggttgagaaaggaggctgcatcatgaacgcttccaccgggtacccatttcccatggtgttcagcagttgcagcaggaaggacttggagaccagcctggagaaaggaatgggggtgtgcctgtttaacctgccggaagtcagggagtctttcgggggccagaagtgtgggaacagatttgtggaagaaggagaggagtgtgactgtggggagccagaggaatgtatgaatcgctgctgcaatgccaccacctgtaccctgaagccggacgctgtgtgcgcacatgggctgtgctgtgaagactgccagctgaagcctgcaggaacagcgtgcagggactccagcaactcctgtgacctcccagagttctgcacaggggccagccctcactgcccagccaacgtgtacctgcacgatgggcactcatgtcaggatgtggacggctactgctacaatggcatctgccagactcacgagcagcagtgtgtcacactctggggaccaggtgctaaacctgcccctgggatctgctttgagagagtcaattctgcaggtgatccttatggcaactgtggcaaagtctcgaagagttcctttgccaaatgcgagatgagagatgctaaatgtggaaaaatccagtgtcaaggaggtgccagccggccagtcattggtaccaatgccgtttccatagaaacaaacatccccctgcagcaaggaggccggattctgtgccgggggacccacgtgtacttgggcgatgacatgccggacccagggcttgtgcttgcaggcacaaagtgtgcagatggaaaaatctgcctgaatcgtcaatgtcaaaatattagtgtctttggggttcacgagtgtgcaatgcagtgccacggcagaggggtgtgcaacaacaggaagaactgccactgcgaggcccactgggcacctcccttctgtgacaagtttggctttggaggaagcacagacagcggccccatccggcaagcagataaccaaggtttaaccataggaattctggtgaccatcctgtgtcttcttgctgccggatttgtggtttatctcaaaaggaagaccttgatacgactgctgtttacaaataagaagaccaccattgaaaaactaaggtgtgtgcgcccttcccggccaccccgtggcttccaaccctgtcaggctcacctcggccaccttggaaaaggcctgatgaggaagccgccagattcctacccaccgaaggacaatcccaggagattgctgcagtgtcagaatgttgacatcagcagacccctcaacggcctgaatgtccctcagccccagtcaactcagcgagtgcttcctcccctccaccgggccccacgtgcacctagcgtccctgccagacccctgccagccaagcctgcacttaggcaggcccaggggacctgtaagccaaacccccctcagaagcctctgcctgcagatcctctggccagaacaactcggctcactcatgccttggccaggaccccaggacaatgggagactgggctccgcctggcacccctcagacctgctccacaatatccacaccaagtgcccagatccacccacaccgcctatattaagtgagaagccgacaccttttttcaacagtgaagacagaagtttgcactatctttcagctccagttggagttttttgtaccaacttttaggattttttttaatgtttaaaacatcattactataagaactttgagctactgccgtcagtgctgtgctgtgctatggtgctctgtctacttgcacaggtacttgtaaattattaatttatgcagaatgttgattacagtgcagtgcgctgtagtaggcatttttaccatcactgagttttccatggcaggaaggcttgttgtgcttttagtattttagtgaacttgaaatatcctgcttgatgggattctggacaggatgtgtttgctttctgatcaaggccttattggaaagcagtcccccaactacccccagctgtgcttatggtaccagatgcagctcaagagatcccaagtagaatctcagttgattttctggattccccatctcaggccagagccaaggggcttcaggtccaggctgtgtttggctttcagggaggccctgtgccccttgacaactggcaggcaggctcccagggacacctgggagaaatctggcttctggccaggaagctttggtgagaacctgggttgcagacaggaatcttaaggtgtagccacaccaggatagagactggaacactagacaagccagaacttgaccctgagctgaccagccgtgagcatgtttggaaggggtctgtagtgtcactcaaggcggtgcttgatagaaatgccaagcacttctttttctcgctgtcctttctagagcactgccaccagtaggttatttagcttgggaaaggtggtgtttctgtaagaaacctactgcccaggcactgcaaaccgccacctccctatactgcttggagctgagcaaatcaccacaaactgtaatacaatgatcctgtattcagacagatgaggactttccatgggaccacaactattttcagatgtgaaccattaaccagatctagtcaatcaagtctgtttactgcaaggttcaacttattaacaattaggcagactctttatgcttgcaaaaactacaaccaatggaatgtgatgttcatgggtatagttcatgtctgctatcattattcgtagatattggacaaagaaccttctctatggggcatcctctttttccaacttggctgcaggaatctttaaaagatgcttttaacagagtctgaacctatttcttaaacacttgcaacctacctgttgagcatcacagaatgtgataaggaaatcaacttgcttatcaacttcctaaatattatgagatgtggcttgggcagcatccccttgaactcttcactcttcaaatgcctgactagggagccatgtttcacaaggtctttaaagtgactaatggcatgagaaatacaaaaatactcagataaggtaaaatgccatgatgcctctgtcttctggactggttttcacattagaagacaattgacaacagttacataattcactctgagtgttttatgagaaagccttcttttggggtcaacagttttcctatgctttgaaacagaaaaatatgtaccaagaatcttggtttgccttccagaaaacaaaactgcatttcactttcccggtgttccccactgtatctaggcaacatagtattcatgactatggataaactaaacacgtgacacaaacacacacaaaagggaacccagctctaatacattccaactcgtatagcatgcatctgtttattctatagttattaagttctttaaaatgtaaagccatgctggaaaataatactgctgagatacatacagaattactgtaactgattacacttggtaattgtactaaagccaaacatatatatactattaaaaaggtttacagaattttatggtgcattacgtgggcattgtctttttagatgcccaaatccttagatctggcatgttagcccttcctccaattataagaggatatgaaccaaaaaaaaaaaaaaaaaaa

SEQ ID NO: 4 - protein, 540 aa, Homo sapiens
  DOMAIN (1)..(540): UniProt P50281 MMP-14
MSPAPRPPRCLLLPLLTLGTALASLGSAQSSSFSPEAWLQQYGYLPPGDLRTHTQRSPQSLSAAIAAMQKFYGLQVTGKADADTMKAMRRPRCGVPDKFGAEIKANVRRKRYAIQGLKWQHNEITFCIQNYTPKVGEYATYEAIRKAFRVWESATPLRFREVPYAYIREGHEKQADIMIFFAEGFHGDSTPFDGEGGFLAHAYFPGPNIGGDTHFDSAEPWTVRNEDLNGNDIFLVAVHELGHALGLEHSSDPSAIMAPFYQWMDTENFVLPDDDRRGIQQLYGGESGFPTKMPPQPRTTSRPSVPDKPKNPTYGPNICDGNFDTVAMLRGEMFVFKERWFWRVRNNQVMDGYPMPIGQFWRGLPASINTAYERKDGKFVFFKGDKHWVFDEASLEPGYPKHIKELGRGLPTDKIDAALFWMPNGKTYFFRGNKYYRFNEELRAVDSEYPKNIKVWEGIPESPRGSFMGSDEVFTYFYKGNKYWKFNNQKLKVEPGYPKSALRDWMGCPSGGRPDEGTEEETEVIIIEVDEEGGGAVSAA

SEQ ID NO: 5 - DNA, 3558 bp, Homo sapiens
  mRNA (1)..(3558): MMP-14 NCBI reference sequence NM_004995.2
cagaccccagttcgccgactaagcagaagaaagatcaaaaaccggaaaagaggagaagagcaaacaggcactttgaggaacaatcccctttaactccaagccgacagcggtctaggaattcaagttcagtgcctaccgaagacaaaggcgccccgagggagtggcggtgcgaccccagggcgtgggcccggccgcggagcccacactgcccggctgacccggtggtctcggaccatgtctcccgccccaagacccccccgttgtctcctgctccccctgctcacgctcggcaccgcgctcgcctccctcggctcggcccaaagcagcagcttcagccccgaagcctggctacagcaatatggctacctgcctcccggggacctacgtacccacacacagcgctcaccccagtcactctcagcggccatcgctgccatgcagaagttttacggcttgcaagtaacaggcaaagctgatgcagacaccatgaaggccatgaggcgcccccgatgtggtgttccagacaagtttggggctgagatcaaggccaatgttcgaaggaagcgctacgccatccagggtctcaaatggcaacataatgaaatcactttctgcatccagaattacacccccaaggtgggcgagtatgccacatacgaggccattcgcaaggcgttccgcgtgtgggagagtgccacaccactgcgcttccgcgaggtgccctatgcctacatccgtgagggccatgagaagcaggccgacatcatgatcttctttgccgagggcttccatggcgacagcacgcccttcgatggtgagggcggcttcctggcccatgcctacttcccaggccccaacattggaggagacacccactttgactctgccgagccttggactgtcaggaatgaggatctgaatggaaatgacatcttcctggtggctgtgcacgagctgggccatgccctggggctcgagcattccagtgacccctcggccatcatggcacccttttaccagtggatggacacggagaattttgtgctgcccgatgatgaccgccggggcatccagcaactttatgggggtgagtcagggttccccaccaagatgccccctcaacccaggactacctcccggccttctgttcctgataaacccaaaaaccccacctatgggcccaacatctgtgacgggaactttgacaccgtggccatgctccgaggggagatgtttgtcttcaaggagcgctggttctggcgggtgaggaataaccaagtgatggatggatacccaatgcccattggccagttctggcggggcctgcctgcgtccatcaacactgcctacgagaggaaggatggcaaattcgtcttcttcaaaggagacaagcattgggtgtttgatgaggcgtccctggaacctggctaccccaagcacattaaggagctgggccgagggctgcctaccgacaagattgatgctgctctcttctggatgcccaatggaaagacctacttcttccgtggaaacaagtactaccgtttcaacgaagagctcagggcagtggatagcgagtaccccaagaacatcaaagtctgggaagggatccctgagtctcccagagggtcattcatgggcagcgatgaagtcttcacttacttctacaaggggaacaaatactggaaattcaacaaccagaagctgaaggtagaaccgggctaccccaagtcagccctgagggactggatgggctgcccatcgggaggccggccggatgaggggactgaggaggagacggaggtgatcatcattgaggtggacgaggagggcggcggggcggtgagcgcggctgccgtggtgctgcccgtgctgctgctgctcctggtgctggcggtgggccttgcagtcttcttcttcagacgccatgggacccccaggcgactgctctactgccagcgttccctgctggacaaggtctgacgcccaccgccggcccgcccactcctaccacaaggactttgcctctgaaggccagtggcagcaggtggtggtgggtgggctgctcccatcgtcccgagccccctccccgcagcctccttgcttctctctgtcccctggctggcctccttcaccctgaccgcctccctccctcctgccccggcattgcatcttccctagataggtcccctgagggctgagtgggagggcggccctttccagcctctgcccctcaggggaaccctgtagctttgtgtctgtccagccccatctgaatgtgttgggggctctgcacttgaaggcaggaccctcagacctcgctggtaaaggtcaaatggggtcatctgctccttttccatcccctgacataccttaacctctgaactctgacctcaggaggctctgggcactccagccctgaaagccccaggtgtacccaattggcagcctctcactactctttctggctaaaaggaatctaatcttgttgagggtagagaccctgagacagtgtgagggggtggggactgccaagccaccctaagaccttgggaggaaaactcagagagggtcttcgttgctcagtcagtcaagttcctcggagatctgcctctgcctcacctaccccagggaacttccaaggaaggagcctgagccactggggactaagtgggcagaagaaacccttggcagccctgtgcctctcgaatgttagccttggatggggctttcacagttagaagagctgaaaccaggggtgcagctgtcaggtagggtggggccggtgggagaggcccgggtcagagccctgggggtgagcctgaaggccacagagaaagaaccttgcccaaactcaggcagctggggctgaggcccaaaggcagaacagccagagggggcaggaggggaccaaaaaggaaaatgaggacgtgcagcagcattggaaggctggggccgggcaggccaggccaagccaagcagggggccacagggtgggctgtggagctctcaggaagggccctgaggaaggcacacttgctcctgttggtccctgtccttgctgcccaggcagcgtggaggggaagggtagggcagccagagaaaggagcagagaaggcacacaaacgaggaatgaggggcttcacgagaggccacagggcctggctggccacgctgtcccggcctgctcaccatctcagtgaggggcaggagctggggctcgcttaggctgggtccacgcttccctggtgccagcacccctcaagcctgtctcaccagtggcctgccctctcgctcccccacccagcccacccattgaagtctccttgggccaccaaaggtggtggccatggtaccggggacttgggagagtgagacccagtggagggagcaagaggagagggatgtcgggggggtggggcacggggtaggggaaatggggtgaacggtgctggcagttcggctagatttctgtcttgtttgtttttttgttttgtttaatgtatatttttattataattattatatatgaattccaaaaaaaaaaaaaaaaaaaaa